(19) United States
(12) Patent Application Publication
(10) Pub. No.: US 2019/0059869 A1
(43) Pub. Date: Feb. 28, 2019

(54) TISSUE RETRACTOR SYSTEMS AND METHODS

(71) Applicant: Cedars-Sinai Medical Center, Los Angeles, CA (US)

(72) Inventors: Pablo Avalos, West Hollywood, CA (US); Doniel Drazin, Los Angeles, CA (US); Clive Svendsen, Pacific Palisades, CA (US); Michael John Baker, Portland, OR (US); Benjamin Thomas Davies, Victoria (CA); Mark Sasha Drlik, Victoria (CA); Julian Snyder Grove, Victoria (CA); Shelby Suckow, Portland, OR (US)

(73) Assignee: Cedars-Sinai Medical Center, Los Angeles, CA (US)

(21) Appl. No.: 15/770,719

(22) PCT Filed: Oct. 28, 2016
(86) PCT No.: PCT/US16/59539

§ 371 Date: Apr. 24, 2018

Related U.S. Application Data
(60) Provisional application No. 62/248,915, filed on Oct. 30, 2015.
(60) Provisional application No. 62/349,942, filed on Jun. 14, 2016.

(51) Int. Cl. A61B 17/02; A61B 90/10; A61B 90/30; A61B 90/50

(52) U.S. Cl. CPC A61B 17/0206; A61B 90/10; A61B 2017/00893; A61B 90/50; A61B 90/30

(57) ABSTRACT

The present invention teaches apparatuses, systems and methods for performing a variety of medical procedures, including those involving introducing one or more substances into a subjects body. In some embodiments, the invention teaches an imaging system that includes an ionizing radiation source and a digital radiation detector that are coupled to opposing blades of a tissue retractor

### FIELD OF THE INVENTION

The present invention generally relates to apparatuses, systems and methods for medical procedures, and especially those that require injecting a substance into a subject's body.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

When physicians are performing procedures on or around certain areas of the body such as the spinal cord, brain, and joints, very precise, controlled, and stable manipulations are often required to avoid patient injury and to optimize outcome. There is a need in the art for apparatuses, systems and methods that will improve the safety, precision, accuracy and efficiency of performing certain medical procedures in those areas, including procedures requiring the injection of one or more medically useful substances.

### SUMMARY OF THE INVENTION

In various embodiments, the invention teaches an apparatus that includes a securing arm that includes a first end, a second end, a long axis, and a short axis; a connecting arm that includes a first end, a second end, a long axis, and a short axis; a positioning arm that includes a first end, a second end, a long axis, and a short axis; and a guiding arm that includes a first end, a second end, a long axis, and a short axis; wherein (1) the first end of the connecting arm is attached to the second end of the securing arm, (2) the second end of the connecting arm is attached to the first end of the positioning arm, (3) the long axis of the connecting arm is perpendicular to the long axis of each of the securing arm and positioning arm, (4) the first end of the securing arm and the second end of the positioning arm can be positioned to extend in substantially the same direction away from the connecting arm, (5) the positioning arm is attached at its second end to the second end of the guiding arm, such that the positioning arm and guiding arm are perpendicular to one another, and (6) the guiding arm can be positioned such that the axis along which its long axis is situated is perpendicular to but does not intersect with the axes along which the long axis of the securing arm and the long axis of the connecting arm are respectively situated. In some embodiments, the securing arm further includes one or more clamps on its first end, and the one or more clamps are configured to attach to an arm of a tissue retractor. In some embodiments, the guiding arm further includes an instrument attaching component configured to slide along the long axis of the guiding arm. In some embodiments, the instrument attaching component includes one or more clamps configured to clamp a medical instrument. In certain embodiments, the sliding motion of the instrument attaching component is controlled by a dial situated at the first end of the guiding arm. In some embodiments, the connecting arm includes elongated nesting elements that allow for telescoping motion in the direction of its long axis, such that the length of the connecting arm can be increased or decreased. In certain embodiments, the positioning arm includes elongated nesting elements that allow for telescoping motion in the direction of its long axis, such that the length of the positioning arm can be increased or decreased. In some embodiments, the telescoping motion of the connecting arm is controlled by rotation of a dial situated at its second end. In certain embodiments, the telescoping motion of the positioning arm is controlled by rotation of a dial situated at its first end. In certain embodiments, the medical instrument is selected from the group consisting of: a cannula, a biopsy needle, a needle, a tube, a cauterization device, a laser, a drill, an endoscope, a guidewire, a fiberoptic device, an electrode, a saw, an ultrasonic device, a spectroscopic device, a camera, an electrical sensor, a thermal sensor, a catheter, a draining tube, and combinations thereof. In some embodiments, the apparatus further includes a side clamp attached to the securing arm, wherein the side clamp is configured to attach to an elongated object. In some embodiments, the securing arm is removably attached to the connecting arm. In various embodiments, the positioning arm is removably attached to the connecting arm and/or the guiding arm. In some embodiments, the side clamp is removably attached to the securing arm. In certain embodiments, the elongated object is a device selected from the group consisting of: a liquid reservoir, a gas reservoir, a pump, an imaging device, and combinations thereof.

In various embodiments, the invention teaches a system. In some embodiments, the system includes any apparatus described above and a tissue retractor attached to the securing arm of the apparatus by one or more clamps of the securing arm. In some embodiments, the system further includes an instrument attached to the instrument attaching component, wherein the instrument is selected from the group consisting of: a cannula, a biopsy needle, a needle, a tube, a cauterization device, a laser, a drill, an endoscope, a guidewire, a fiberoptic device, an electrode, a saw, an ultrasonic device, a spectroscopic device, a camera, an electrical sensor, a thermal sensor, a catheter, a draining tube, and combinations thereof. In some embodiments, the instrument includes a cannula with a needle situated at the end thereof. In some embodiments, the cannula and needle are configured to inject cells into a region of interest in a subject's body. In various embodiments, the cannula contains a quantity of neural progenitor cells. In some embodiments, the neural progenitor cells express glial cell line derived neurotrophic factor. In certain embodiments, the region of interest is the subject's spine. In some embodiments, the system further includes a liquid reservoir and a pump connected thereto, wherein the liquid reservoir and pump are attached to the side clamp.

In various embodiments, the invention teaches a method for performing a surgical procedure on a subject. In some embodiments, the method includes attaching any apparatus described herein above to an arm of a tissue retractor that is engaged in an incision in the subject's body, and guiding a medical instrument attached to the guiding arm of the apparatus through the incision in the subject's body. In certain embodiments, the medical instrument is a cannula with a needle situated at the end thereof. In some embodiments, the cannula and needle are configured to inject cells into a region of interest in the subject's body. In some embodiments, the region of interest is the subject's spine. In some embodiments, the cells are neural progenitor cells. In some embodiments, the subject has been diagnosed with amyotrophic lateral sclerosis (ALS). In various embodiments, the method further includes performing imaging of the region of interest in the subject's body. In some embodiments, the imaging performed is selected from the group consisting of computed tomography (CT), magnetic resonance imaging (MM), ultrasound, and combinations thereof. In some embodiments, the method further includes injecting neural progenitor cells expressing glial cell line derived neurotrophic factor into the subject's spine.

In some embodiments, a floating cannula system for precision injections is disclosed, the system including (1) a base cannula with a proximal end, a distal end, and a lumen; (2) a floating cannula at least partially contained inside the lumen of the base cannula and having a lumen; the floating cannula having a proximal end and a distal end that extend beyond the proximal end and distal end of the base cannula, wherein the floating cannula is configured to move in a direction along a longitudinal axis of the base cannula and simultaneously move with respect to the base cannula. Also disclosed in connection with the system is a distal stopper connected to the distal end of the floating cannula that is configured to prevent movement of the distal stopper in the proximal direction past the distal end of the base cannula. A proximal stopper on the proximal end of the floating cannula configured to prevent movement of the proximal stopper in the distal direction past the proximal end of the base cannula, where the distance from the proximal stopper to the distal stopper is greater than the distance between the proximal and distal ends of the base cannula. Also disclosed is a needle connected to the distal end of the floating cannula, and a delivery tube connected to the needle and partially contained inside the lumen of the floating cannula and/or the lumen of the base cannula.

In some embodiments, a pair of support tabs may be connected to the base cannula. In some embodiments, a connector is removably attached (or permanently attached) to the support tabs. In some embodiments, the connector is removably attached (or permanently attached) to the guiding arm of a stereotactic device, including any stereotactic device described herein. In some embodiments, the delivery tube is connected to an external pump and reservoir containing a substance to inject into the tissue site. In some embodiments, the needle includes a tissue stopper. In certain embodiments, the positions of the distal and/or proximal stoppers on the floating cannula may be movable. In some embodiments, the support tabs have thumb grips. In some embodiments, the connector includes spaces or indentations that are configured to fit an end of the one or more support tabs, and a corresponding tab lock that locks the one or more tabs into the spaces or indentations. In some embodiments, the base cannula is positioned at least partially within the lumen of the floating cannula (reversing the orientation described above).

In some embodiments, the invention includes a method of manufacturing a cannula that may include manufacturing a base cannula and a floating cannula, wherein the floating cannula is manufactured so the diameter to the outside edges is slightly smaller than the diameter of the lumen of the base cannula or vice versa. This will allow the floating cannula (or base cannula when reversed as indicated above) to slide along the base cannula and restrict its movement in the lateral direction, while still allowing movement in the direction of the longitudinal axes of the cannulas. In some embodiments, the floating cannula will be manufactured to be significantly longer than the base cannula, for example two centimeters longer, three centimeters, or any other suitable amount for a particular procedure. In some embodiments, the base cannula and floating cannula may be manufactured from surgical grade metal, plastic or other suitable materials.

In some embodiments, one, two, or more support tabs may be attached to the base cannula. The support tabs may be manufactured from plastic or another suitable material, and may include thumb tabs. In some embodiments, the support tabs will include holes through which the base cannula may be inserted, and then adhesive may be applied to fix the support tabs in place. In other embodiments, the support tabs may be welded or otherwise affixed onto the base cannula.

In some embodiments the floating cannula will be inserted into the base cannula and then stoppers will be fixed to both ends of the floating cannula that protrude from the base cannula. In some embodiments, the stoppers will be positioned so that the base cannula has a few centimeters of travel between a proximal stopper and a distal stopper, or put differently, the floating cannula will be able to move a few centimeters in the distal and proximal direction before the base cannula's proximal or distal rims (located at its ends) restrict further movement. In some embodiments, the stoppers are positioned to allow several or many inches of travel, depending upon the anatomical target and procedure.

In some embodiments, a delivery tube will be threaded through the entire floating cannula system and fixed to (such that it is in fluid communication with) a hollow needle located at the distal end of the floating cannula. In some embodiments, the delivery tube will include a hollow needle, or a hollow needle will be fixed to it, and then it will be threaded through and fixed to a collar or needle fixture at the distal end of the floating cannula. The delivery tube may be made from any suitable plastic, metal or other material to delivery substances for injection. For instance, standard PTFE tubing of the appropriate thickness may be used.

In some embodiments, the invention discloses a method for injecting a substance into a subject using a floating cannula system. In some embodiments, the method includes providing a floating cannula system that includes a floating cannula, a base cannula, a distal stopper, a hollow needle, a tissue stopper and a delivery tube. The method may further include advancing the floating cannula system towards a tissue site on the subject, until the hollow needle contacts the tissue site; and then advancing the floating cannula system farther in the same direction until the distal stopper prevents the floating cannula from moving with respect to the base cannula; and then advancing the floating cannula system farther in the same direction until the hollow needle is inserted into the tissue site; and then advancing the floating cannula system farther in the same direction until the tissue stopper contacts the tissue site. In some embodiments, the method further includes retracting the base cannula from the tissue site such that there is space between the distal stopper on the floating cannula and the distal end of the base cannula, thus allowing the floating cannula to travel along the longitudinal axis of the cannulas in response to the subject's movement along that axis (e.g. bucking or breathing), while allowing the hollow needle to remaining engaged in the tissue site. In some embodiments, the method further includes injecting a substance through the delivery tube and hollow needle and into the tissue site. In some embodiments, the floating cannula system is advanced towards the tissue site by advancing a guiding arm of a stereotactic device to which the floating cannula/system is attached towards the tissue site (as demonstrated in the drawings and described herein).

In various embodiments, the invention teaches a syringe pump system. In some embodiments, the term “syringe pump system” is used broadly to define a system that incorporates a syringe pump, along with other apparatuses and systems connected thereto (e.g. cannulas and delivery tubes). In some embodiments, the syringe pump system includes a motor assembly, including (a) a housing, including a first end and a second end, (b) a motor, and (c) a rotatable drive shaft, wherein the motor is configured to cause the rotatable drive shaft to rotate, and the motor and rotatable drive shaft are at least partly contained within the housing. In some embodiments, the syringe pump system further includes a carpule assembly, including (a) a first end including an elongated inlet port, (b) a second end including an elongated outlet port, and (c) a chamber disposed between and in fluid communication with the elongated inlet port and the elongated outlet port. In some embodiments, the syringe pump system further includes an elongated plunger, which includes (a) a receiving end, (b) a body, and (c) a pushing end, wherein (1) the elongated plunger is configured to nest within the elongated inlet port, (2) the pushing end of the elongated plunger is configured to form a substantially fluid-tight seal with the chamber, and wherein the rotatable drive shaft is configured to apply a drive force to the receiving end of the elongated plunger, either directly, or indirectly through an intervening shaft, such that the elongated plunger can be pushed in the direction of the outlet port, thereby expelling any liquid in the chamber. In some embodiments, the syringe pump system further includes a coupling collar that includes a first end and a second end, wherein the first end of the coupling collar is configured to interact with and removably connect to the second end of the housing, and wherein the second end of the coupling collar is configured to interact with and removably connect to the first end of the carpule assembly. In some embodiments, the syringe pump system further includes a delivery tube that includes a first end and a second end, wherein the first end of the delivery tube is connected to and in fluid communication with the second end of the carpule assembly. In some embodiments, the second end of the delivery tube is connected to and in fluid communication with a cannula that includes a hollow needle.

In various embodiments, the second end of the delivery tube described above is connected to and in fluid communication with a floating cannula system configured to inject a substance into a subject. In some embodiments, the floating cannula system includes a base cannula that includes a proximal end, a distal end, and a lumen; a floating cannula that includes a lumen, wherein (1) the floating cannula is configured to be at least partially contained inside the lumen of the base cannula, (2) the floating cannula includes a proximal end and a distal end that extend farther proximally and distally than the proximal end and distal end of the base cannula when engaged therein, and (3) the floating cannula is configured to move in a direction along a longitudinal axis of the base cannula when engaged therein; a distal stopper connected to the distal end of the floating cannula, wherein the distal stopper is configured and positioned to prevent movement of the distal stopper in the proximal direction past the distal end of the base cannula when the floating cannula is engaged in the base cannula; a proximal stopper connected to the proximal end of the floating cannula, wherein the proximal stopper is configured and positioned to prevent movement of the proximal stopper in the distal direction past the proximal end of the base cannula, and wherein the distance from the proximal stopper to the distal stopper is greater than the distance between the proximal and distal ends of the base cannula; and a hollow needle connected to the distal end of the floating cannula. In some embodiments, the second end of the delivery tube described above is connected to the hollow needle, and at least part of the length of the delivery tube is contained inside the lumen of the floating cannula and the lumen of the base cannula. In certain embodiments, a pair of support tabs is connected to the base cannula. In some embodiments, the syringe pump system further includes a connector removably attached to the support tabs of the cannula system. In some embodiments, the syringe pump system further includes a stereotactic device that includes a guiding arm configured to be advanced toward and retracted from a tissue site (as described herein), and the connector is removably (or permanently) attached to the guiding arm of the stereotactic device. In some embodiments of the syringe pump system, the hollow needle of the floating cannula system includes a tissue stopper. In some embodiments of the syringe pump system, the positions of the distal and/or proximal stoppers on the floating cannula may be changed. In some embodiments of the syringe pump system, the support tabs of the floating cannula system include thumb grips. In certain embodiments, the connector includes one or more indentations configured to closely fit an end of one or more of the support tabs. In certain embodiments of the syringe pump system, the connector includes a tab lock that locks one or more of the support tabs in place in the one or more indentations.

In various embodiments, the invention teaches a method for injecting a fluid substance into a subject. In some embodiments, the method includes (1) providing a syringe pump system, wherein the syringe pump system includes: a motor assembly, including (a) a housing, which includes a first end and a second end, (b) a motor, and (c) a rotatable drive shaft, wherein the motor is configured to cause the rotatable drive shaft to rotate, and the motor and rotatable drive shaft are at least partly contained within the housing; a carpule assembly, including (a) a first end including an elongated inlet port, (b) a second end including an elongated outlet port, and (c) a chamber disposed between and in fluid communication with the elongated inlet port and the elongated outlet port; an elongated plunger, including (a) a receiving end, (b) a body, and (c) a pushing end, wherein (1) the elongated plunger is configured to nest within the elongated inlet port, (2) the pushing end of the plunger is configured to form a substantially fluid-tight seal with the chamber, and wherein the rotatable drive shaft is configured to apply a drive force to the receiving end of the plunger, either directly, or indirectly through an intervening shaft, such that the plunger can be pushed in the direction of the outlet port, thereby expelling any liquid in the chamber. In some embodiments, the syringe pump system further includes a floating cannula system, including a delivery tube that includes a first delivery tube end and a second delivery tube end, wherein (1) the first delivery tube end is connected to and in fluid communication with the second end of the carpule assembly, and (2) the second delivery tube end is connected to and in fluid communication with a hollow needle. In some embodiments, the syringe pump system further includes a medically useful fluid substance located within the chamber of the carpule assembly. In some embodiments, the method includes (1) inserting a portion of the hollow needle into the subject, and (2) pumping the medically useful fluid substance out of the chamber of the carpule assembly, through the delivery tube and hollow needle, and into the subject. In some embodiments the hollow needle is inserted into the spinal cord of the subject.

In certain embodiments, the cannula system incorporated into the syringe pump system is a floating cannula system that includes a base cannula including a proximal end, a distal end, and a lumen; a floating cannula including a lumen, wherein (a) the floating cannula is configured to be at least partially contained inside the lumen of the base cannula, (b) the floating cannula includes a proximal end and a distal end that extend farther proximally and distally than the proximal end and distal end of the base cannula when engaged therein, and (c) the floating cannula is configured to move in a direction along a longitudinal axis of the base cannula when engaged therein; a distal stopper connected to the distal end of the floating cannula, wherein the distal stopper is configured and positioned to prevent movement of the distal stopper in the proximal direction past the distal end of the base cannula, when the floating cannula is engaged in the base cannula; a proximal stopper connected to the proximal end of the floating cannula, wherein (1) the proximal stopper is configured and positioned to prevent movement of the proximal stopper in the distal direction past the proximal end of the base cannula; (2) the distance from the proximal stopper to the distal stopper is greater than the distance between the proximal and distal ends of the base cannula; (3) a hollow needle is connected to the distal end of the floating cannula; (4) a delivery tube is connected to the hollow needle, and (5) at least part of the length of the delivery tube is contained inside the lumen of the floating cannula and/or the lumen of the base cannula. In certain embodiments, the floating cannula system incorporated within the syringe pump system further includes a pair of support tabs connected to the base cannula. In certain embodiments, the floating cannula system further includes a connector removably attached to the support tabs. In certain embodiments, the syringe pump system further includes a stereotactic device including a guiding arm configured to be advanced toward or retracted from a tissue site, and the connector is removably attached to the guiding arm of the stereotactic device. In certain embodiments, the hollow needle of the floating cannula system includes a tissue stopper. In some embodiments, the positions of the distal and/or proximal stoppers on the floating cannula may be changed. In certain embodiments, the support tabs of the floating cannula system include thumb grips. In some embodiments, the connector of the floating cannula system includes one or more indentations configured to closely fit an end of one or more of the support tabs. In certain embodiments, the substance injected into the subject's spinal cord includes cells. In various embodiments, the cells are neural progenitor cells. In certain embodiments, the neural progenitor cells express glial cell line derived neurotrophic factor. In some embodiments, the subject is a human who has been diagnosed with amyotrophic lateral sclerosis (ALS).

In some embodiments, the invention teaches a kit that includes any of the syringe pump systems described above, and instructions for the use thereof to inject a medically useful substance into a subject.

In various embodiments, the invention teaches an imaging system that includes (a) a tissue retractor including a first blade and a second blade; (b) a radiation source coupled to the first blade of the tissue retractor; and (c) a radiation detector coupled to the second blade of the tissue retractor. In some embodiments, the imaging system further includes a collimating device configured to collimate the radiation emitted from the radiation source. In some embodiments, the radiation source is an ionizing radiation source that includes argon. In certain embodiments, the radiation detector is a digital radiation detector. In certain embodiments, each of the retractor blades is connected to a retractor arm. In some embodiments, the imaging system further includes a stereotactic device attached to one of the retractor arms, and the stereotactic device includes an elongated guiding arm capable of advancing towards and retracting from a tissue site of a subject. In some embodiments, the imaging system further includes a cannula system, and (1) the cannula system is attached to the guiding arm of the stereotactic device, and (2) the cannula system includes a hollow needle and a tube connected thereto. In some embodiments, the imaging system further includes a syringe pump, and the syringe pump is operably connected to and in fluid communication with the tube of the cannula system. In certain embodiments, the syringe pump is attached to the stereotactic device. In some embodiments, the imaging system further includes a computing system operably connected to the ionizing radiation source and/or the digital radiation detector. In some embodiments, the computing system includes a processor configured to process data acquired by utilizing the ionizing radiation source and the digital radiation detector. In certain embodiments, the computing system is wirelessly connected to the ionizing radiation source and/or the digital radiation detector. In some embodiments, the computing system is connected to the ionizing radiation source and/or the digital radiation detector through one or more wires.

In various embodiments, the invention teaches a method that includes (1) providing an imaging system that includes (a) a tissue retractor including a first blade and a second blade; (b) a radiation source coupled to the first blade of the tissue retractor; and (c) a radiation detector coupled to the second blade of the tissue retractor; (2) positioning each of the first and second retractor blades of the imaging system on opposing sides of an anatomical structure of a subject; and (3) imaging the anatomical structure by operating the radiation source and the radiation detector. In certain embodiments, the imaging system further includes a collimating device configured to collimate the radiation emitted from the radiation source. In some embodiments, the radiation source of the imaging system is an ionizing radiation source that includes argon. In certain embodiments, the radiation detector of the imaging system is a digital radiation detector. In some embodiments, each of the retractor blades of the imaging system is connected to a retractor arm. In certain embodiments, the imaging system further includes a stereotactic device attached to one of the retractor arms, and the stereotactic device includes an elongated guiding arm capable of advancing into and retracting away from a tissue site on a subject. In some embodiments, the imaging system further includes a cannula. In some embodiments, the cannula (1) is attached to the guiding arm of the stereotactic device, and (2) includes a hollow needle and a tube connected thereto. In some embodiments, the imaging system further includes a syringe pump. In some embodiments, (1) the syringe pump is operably connected to and in fluid communication with the tube of the cannula, and (2) the syringe pump is attached to the stereotactic device. In certain embodiments, the imaging system further includes a computing system operably connected to the ionizing radiation source and/or the digital radiation detector, and the computing system includes a processor configured to process data acquired by utilizing the ionizing radiation source and digital radiation detector. In some embodiments, the computing system is wirelessly connected to the ionizing radiation source and/or the digital radiation detector. In certain embodiments, the computing system is connected to the ionizing radiation source and/or digital radiation detector through one or more wires. In certain embodiments, the anatomical structure is the subject's spinal cord. In some embodiments, the method further includes introducing the needle of the cannula system into the subject's spinal cord by advancing the guiding arm of the stereotactic device toward the subject's spinal cord. In some embodiments, the method further includes operating the syringe pump to pump a composition that includes cells into the subject's spinal cord through the needle. In some embodiments, the cells include neural progenitor cells. In certain embodiments, the neural progenitor cells express glial cell line derived neurotrophic factor. In some embodiments, the subject has been diagnosed with amyotrophic lateral sclerosis.

In various embodiments, the invention teaches a kit that includes any of the imaging systems described herein and instructions for the use thereof to image an anatomical structure of a subject.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1A depicts, in accordance with an embodiment of the invention, stereotactic apparatus 100. Stereotactic apparatus 100 is clamped to arm 301 of tissue retractor 300. Cylindrical object 400 is fastened to stereotactic apparatus 100 by side clamp 6000. FIG. 1B depicts stereotactic apparatus 100 without attachment to a tissue retractor. FIG. 1C depicts stereotactic apparatus 200. FIG. 1D depicts stereotactic apparatus 100 attached to cylindrical object 400 and tissue retractor 300. Instrument 7000 is shown attached to guiding arm 1000 of stereotactic apparatus 100, and extending downward along the z-axis between the arms of tissue retractor 300.

FIG. 2A depicts, in accordance with an embodiment of the invention, stereotactic apparatus 100. Tissue retractor 300 and cylindrical object 400 are shown. FIG. 2B depicts an alternate view of stereotactic apparatus 100. FIG. 2C depicts an alternate view of stereotactic apparatus 200.

FIG. 3 depicts, in accordance with an embodiment of the invention, a partially exploded view of stereotactic apparatus 100.

FIG. 4 depicts, in accordance with an embodiment of the invention, a partially exploded view of stereotactic apparatus 100.

FIG. 5 depicts, in accordance with an embodiment of the invention, loosening knob 114 allows for adjustment of the position of positioning arm 2000 along the x-axis.

FIG. 6 depicts, in accordance with an embodiment of the invention, loosening screw 135 allows for adjustment of the position of positioning arm 2000 along the y-axis.

FIG. 7 depicts, in accordance with an embodiment of the invention, loosening knob 130 allows for adjustment of the position of cylindrical object 400 along the x-axis.

FIG. 8 depicts, in accordance with an embodiment of the invention, loosening of knob 114 allows for rotation of positioning arm 2000 around the x-axis and associated motion of guiding arm 1000 along the y-z plane.

FIG. 9 depicts, in accordance with an embodiment of the invention, loosening screw 135 allows for rotation of cross clamp 132 around the y-axis, and associated motion of guiding arm 1000 along the x-z plane.

FIG. 10 depicts, in accordance with an embodiment of the invention, rotating dial 116 causes telescoping of inner nesting element 112 of positioning arm 2000. FIG. 10 also shows rotating dial 101 causes motion of instrument attachment component 107 along the z-axis.

FIG. 11 depicts, in accordance with an embodiment of the invention, rotating dial 131 causes telescoping motion of inner nesting element 119 of connecting arm 3000.

FIG. 12 depicts, in accordance with an embodiment of the invention, a partially exploded view of connecting arm 3000. Arrows labeled “14A” indicate the cross section represented in FIG. 14A.

FIG. 13 depicts, in accordance with an embodiment of the invention, an exploded view of a portion of connecting arm 3000.

FIG. 14A depicts, in accordance with an embodiment of the invention, a cross-sectional view of the long axis of connecting arm 3000. FIG. 14B depicts a cross-sectional view of the short axis of connecting arm 3000.

FIG. 15 depicts, in accordance with an embodiment of the invention, a partially exploded view of positioning arm 2000. Arrows labeled “17A” indicate the cross section represented in FIG. 17A.

FIG. 16 depicts, in accordance with an embodiment of the invention, a partially exploded view of a portion of positioning arm 2000.

FIG. 17A depicts, in accordance with an embodiment of the invention, a cross-sectional view of the long axis of positioning arm 2000. FIG. 17B depicts, in accordance with an embodiment of the invention, a cross sectional view of the short axis of positioning arm 2000.

FIG. 18 depicts, in accordance with an embodiment of the invention, an exploded view of guiding arm 1000. Arrows labeled “19” indicate the cross section represented in FIG. 19.

FIG. 19 depicts, in accordance with an embodiment of the invention, a cross-sectional view of the long axis of guiding arm 1000.

FIG. 20 depicts, in accordance with an embodiment of the invention, an exploded view of side clamp 6000, and it's attachment to securing arm 4000.

FIG. 21 depicts, in accordance with an embodiment of the invention, an alternate exploded view of securing arm 4000.

FIG. 22 depicts, in accordance with an embodiment of the invention, side clamp 6000.

FIG. 23 depicts, in accordance with an embodiment of the invention, a perspective view of a floating cannula system 8000.

FIG. 24 depicts, in accordance with an embodiment of the invention, a perspective view of a floating cannula system 8000 attached to connector 420.

FIG. 25 depicts, in accordance with an embodiment of the invention, an exploded view of a floating cannula system 8000 and connector 420.

FIG. 26 depicts, in accordance with an embodiment of the invention, a perspective and exploded view of a floating cannula system 8000 attached to connector 420.

FIG. 27 depicts, in accordance with an embodiment of the invention, a side view of a floating cannula system 8000 attached to connector 420.

FIG. 28 depicts, in accordance with an embodiment of the invention, a perspective view of a floating cannula system 8000 prior to attachment to a connector 420 and stereotactic device 100.

FIG. 29 depicts, in accordance with an embodiment of the invention, a perspective view of a floating cannula system 8000 and support tabs 402 that have been mounted on pins 424 of connector 420 and stereotactic device 100.

FIG. 30 depicts, in accordance with an embodiment of the invention, a perspective view of a floating cannula system 8000 attached to a connector 420 and stereotactic device 100 after the support tabs 402 have been rotated into spaces or indentations 422.

FIG. 31 depicts, in accordance with an embodiment of the invention, a partially exploded view of syringe pump 9000.

FIG. 32 depicts, in accordance with an embodiment of the invention, a cross-sectional and partially exploded view of a portion of syringe pump 9000.

FIG. 33 depicts, in accordance with an embodiment of the invention, a cross-sectional view of a portion of syringe pump 9000.

FIG. 34 depicts, in accordance with an embodiment of the invention, syringe pump 9000 can be positioned in side clamp 6000 of stereotactic device 100.

FIG. 35 depicts, in accordance with an embodiment of the invention, syringe pump 9000 engaged in side clamp 6000 of stereotactic device 100.

FIG. 36 depicts, in accordance with an embodiment of the invention, syringe pump 9000 connected to floating cannula 8000 through delivery tube 7000. The floating cannula 8000 is shown connected to the guiding arm of stereotactic device 100.

FIG. 37 depicts, in accordance with an embodiment of the invention, syringe pump 9000 can be connected to floating cannula 8000 through tube 10000. Tube 10000 terminates in coupler/connector 10001 on one end, which couples tube 10000 to syringe pump 9000. On the other end, tube 10000 is connected to delivery tube 7000 through male Luer lock fitting 10002 and female Luer lock fitting 10003. The floating cannula 8000 is shown connected to the guiding arm of stereotactic device 100.

FIG. 38 depicts, in accordance with an embodiment of the invention, an ionizing radiation source and detector coupled to the blades of a tissue retractor. The figure illustrates the positions of the ionizing radiation source and detector relative to a section of the spine that is being imaged.

FIG. 39 depicts, in accordance with an embodiment of the invention, a specific orientation of an ionizing radiation source and a detector coupled to the blades of a tissue retractor.

FIGS. 40A-40F depict, in accordance with an embodiment of the invention, a retractor device (including blades) that can be utilized in conjunction with the radiation sources and detectors depicted in FIGS. 38 and 39, as well as other embodiments of radiation sources and detectors described herein, and stereotactic devices described herein.

FIG. 41 depicts, in accordance with an embodiment of the invention, stereotactic apparatus 100. Stereotactic apparatus 100 is shown clamped to fixed arm 18004 of tissue retractor 18000.

FIG. 42A depicts, in accordance with an embodiment of the invention, stereotactic apparatus 100, and the location of attachment to tissue retractor 18000.

FIG. 43A depicts, in accordance with an embodiment of the invention, a top-down view of tissue retractor 10000, with curved tissue retractor blades 10001a and 10001b in close proximity. FIG. 43B depicts a top-down view of tissue retractor 10000, with curved tissue retractor blades 10001a and 10001b separated.

FIG. 44A depicts, in accordance with an embodiment of the invention, a top-down view of tissue retractor blade system 11000 with tissue retractor blades 11001a and 11001b that slide along tracks 11002a and 11002b. Tissue retractor blades 11001a and 10001b are shown in close proximity in FIG. 44A and separated in the top-down view shown in FIG. 44B.

FIG. 45A depicts, in accordance with an embodiment of the invention, a top-down view of tissue retractor blade system 12000 with tissue retractor blades 12001a and 12001b and hinged folding bars 12002a and 12002b. Tissue retractor blades 12001a and 12001b are shown in close proximity in FIG. 45A and separated in the top-down view shown in FIG. 45B.

FIG. 46A depicts, in accordance with an embodiment of the invention, a top-down view of tissue retractor system 13000 with tissue retractor blades 13001a and 13001b and medial-lateral sliding components 13002a, 13002b, 13003a, 13003b, 13003c, and 13003d. Tissue retractor blades 13001a and 13001b are shown in close proximity (without a medial bridge engaged) in FIG. 46A and separated by medial bridges 13002a and 13002b in FIG. 46B.

FIG. 47A depicts, in accordance with an embodiment of the invention, a top-down view of tissue retractor system 14000 with tissue retractor blades 14001a and 14001b. Tissue retractor blades 14001a and 14001b are shown in close proximity (overlapping) in FIG. 47A and separated in FIG. 47B.

FIG. 48A depicts, in accordance with an embodiment of the invention, a top-down view of tissue retractor system 15000 with interlocking tissue retractor blades 15001a and 15001b, which include sets of finger projections 15001c, 15001d, 15001e, 15001f (shown in FIG. 48B). Tissue retractor blades 15001a and 15001b are shown in close proximity in FIG. 48A and separated in FIG. 48B. FIG. 48C shows a side view of tissue retractor blades 15001a and 15001b3 with interlocking ‘fingers.’ FIG. 48D shows a side view of tissue retractor blades 15001a and 15001b in which tissue retractor blades 15001a and 15001b are separated.

FIG. 49A depicts, in accordance with an embodiment of the invention, a top-down view of tissue retractor system 16000 with tissue retractor blades 16001a and 16001b and contoured sections 16002a and 16002b. FIG. 49B shows a perspective view of tissue retractor system 16000, in which tissue retractor blades 16001a and 16001b are in close proximity. FIG. 49C shows a perspective view of tissue retractor system 16000, in which tissue retractor blades 16001a and 16001b are separated.

FIG. 50 depicts, in accordance with an embodiment of the invention, a perspective view 10 of tissue retractor system 17000 with tissue retractor blades 17001a and 17001b, which include openings 17001c and 17001d, as shown.

FIG. 51A depicts, in accordance with an embodiment of the invention, tissue retractor system 18000 in a closed configuration, in which opposing tissue retractor blades 18001a and 18001b are interlocked/overlapping. FIG. 51B depicts, in accordance with an embodiment of the invention, an exploded view of tissue retractor system 18000.

FIG. 52 depicts, in accordance with an embodiment of the invention, a perspective view of tissue retractor system 18000.

FIG. 53A depicts, in accordance with an embodiment of the invention, a side view of tissue retractor 18000, in which guide pin 18002b and rotation stops 18030a and 18031a are shown. FIG. 53B depicts a side view of tissue retractor system 18000, in which tissue undercut teeth 18022a and 18022b, tissue engagement ridges 18021a, and blade nesting slot 18031a are shown.

FIG. 54 depicts, in accordance with an embodiment of the invention, a view of blade nesting slot 18031a of tissue retractor system 18000.

FIG. 55 depicts, in accordance with an embodiment of the invention, tissue 20000 encroaching between the blades of tissue retractor 10000.

FIG. 56 depicts, in accordance with an embodiment of the invention, tissue retractor 10000 with medial lateral retractor blades 21000a and 21000b attached thereto and preventing encroachment of tissue.

FIG. 57 depicts, in accordance with an embodiment of the invention, a perspective view of stabilizing arms attached to tissue retractor system 18000; and

FIG. 58 depicts, in accordance with an embodiment of the invention, a perspective view of stabilizing arms attached to tissue retractor system 18000.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

The terms “patient” and “subject” are used interchangeably herein. These terms are intended to include all animal subjects, including mammals. Human patients/subjects are intended to be within the scope of the patients/subjects treated using the various embodiments of the inventive systems, apparatuses and methods described herein.

As used herein, the terms “anatomical feature” and “anatomical structure” include any tissue or collection of tissues found on or in a subject's body.

As demonstrated herein, in some embodiments the invention discloses novel stabilizing apparatuses, cannula systems and apparatuses, syringe pump systems and apparatuses, and methods of use thereof. In some embodiments, the invention discloses imaging systems and methods that can be used alone or in conjunction with the aforementioned apparatuses, systems and methods. While one of skill in the art would readily appreciate that there are many possible applications of the systems and apparatuses described herein, certain embodiments are especially useful for procedures performed on or around the spinal cord, including delivery of cutting edge cellular and molecular therapies thereto.

Although numerous embodiments of stereotactic apparatuses are described herein, there are certain features common to all of them. First, each stereotactic apparatus includes one or more components that make up a “securing section” capable of stably connecting to an arm of a tissue retracting device. The second feature common to each of the stereotactic apparatuses described herein is a “positioning section,” which includes one or more components capable of positioning an instrument over a desired location in a subject's body. The third common feature is a “connecting section,” which serves to operably connect the positioning section and the securing section. A fourth common feature is a “guiding section,” which can be used to guide an instrument into or remove an instrument from a subject's body.

Provided below are descriptions of various components, combinations of components, and configurations of components relative to one another that can be used to arrive at each of the common sections described above. Additional features that can be added to the stereotactic apparatus are also described.

### Securing Section

In some embodiments, the securing section of the stereotactic apparatus is configured to removably attach to an arm of a tissue retractor. Removable attachment can be accomplished in any of a number of ways, using a wide range of components and combinations thereof. Merely by way of non-limiting examples, the securing section could attach to the arm of a tissue retractor by using one or more clasps, one or more clamps, one or more magnets, one or more screws, one or more pins, one or more slot and groove arrangements, one or more straps, combinations thereof and the like. Therefore, each of these components, and modified versions thereof, are within the scope of the invention. It is further contemplated that the attaching portion of the apparatus could be configured to attach to any of a variety of types of equipment that might be found in a setting in which a medical procedure is performed, including, but in no way limited to a table, a lamp, a brace, a tray, imaging equipment, and the like. It is also contemplated that the device could be configured for use in a non-surgical setting, in which it may be used to perform any objective that requires the use of precision guidance. It is also contemplated that the device could be scaled appropriately for such objectives.

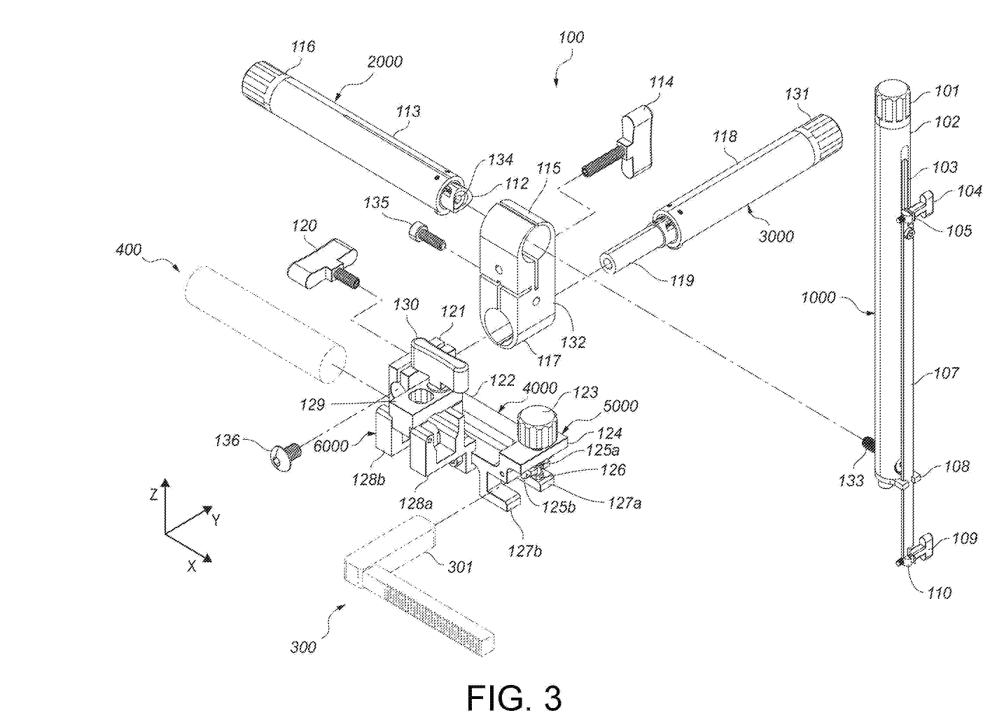

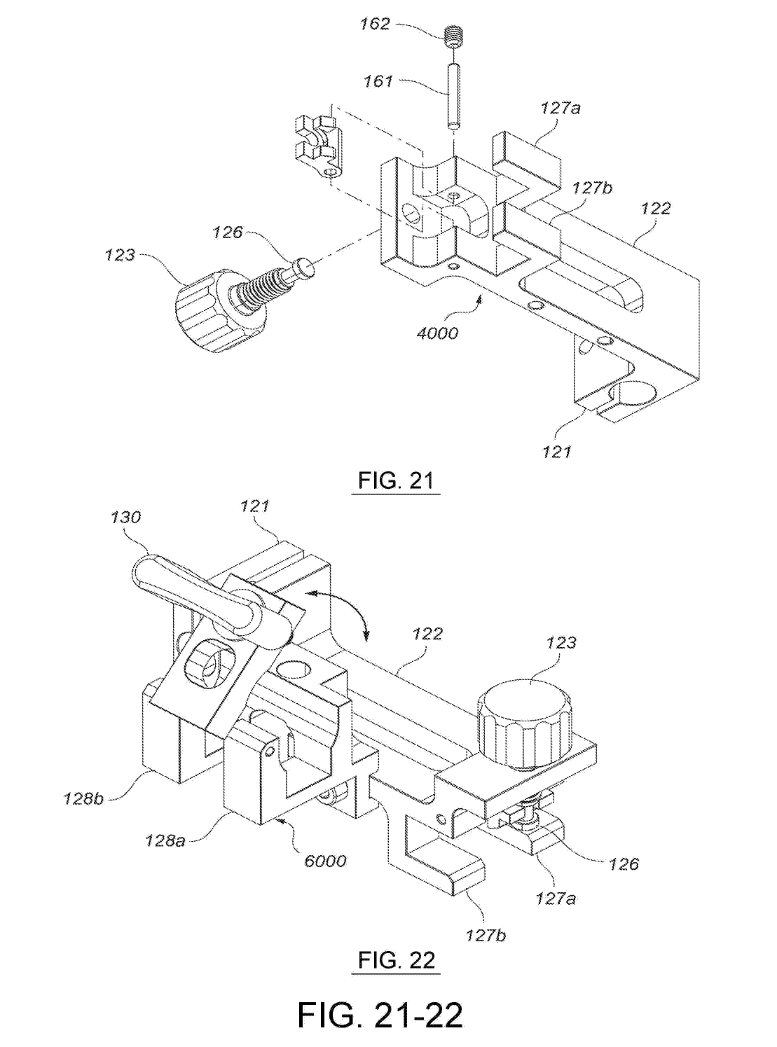

In some embodiments, a clamping mechanism is incorporated on the securing arm, and used to attach the stereotactic apparatus to the arm of a tissue retractor. One of skill in the art would readily appreciate that numerous types of clamping mechanisms are suitable to accomplish this function. One non-limiting example is depicted in FIG. 3, which shows clamping mechanism 5000 of securing arm 4000 can be used to clamp arm 301 of tissue retractor 300 (partially shown). A more detailed view of the clamping components of this particular embodiment is shown in FIG. 21, and the individual components (and their functions) are thoroughly described in the examples section.

Importantly, the clamping mechanism shown in FIG. 21 can be used to securely and removably attach a stereotactic apparatus (including stereotactic apparatus 100) to the arm of a number of different types of tissue retractors. Non-limiting examples of retractors to which the clamping mechanism can attach include the Mast Quadrant Retractor System (Medtronic), the MARS Retractor System (Globus Medical), the Spyder Retractor System (Aesculap), the Ravine Retractor System (K2M), the Synframe Retractor System (DePuy Synthes), and the Luxor Retractor System (Stryker). One of skill in the art would readily appreciate that any retractor with one or more arms similar to those retractors described above could also be used in conjunction with the inventive stereotactic apparatuses described herein. One of skill in the art would further appreciate that the alternative attaching mechanisms described above would allow for the attachment of the securing section of an apparatus to one or more arms of alternative retractor devices that are not specifically listed above.

### Positioning Section

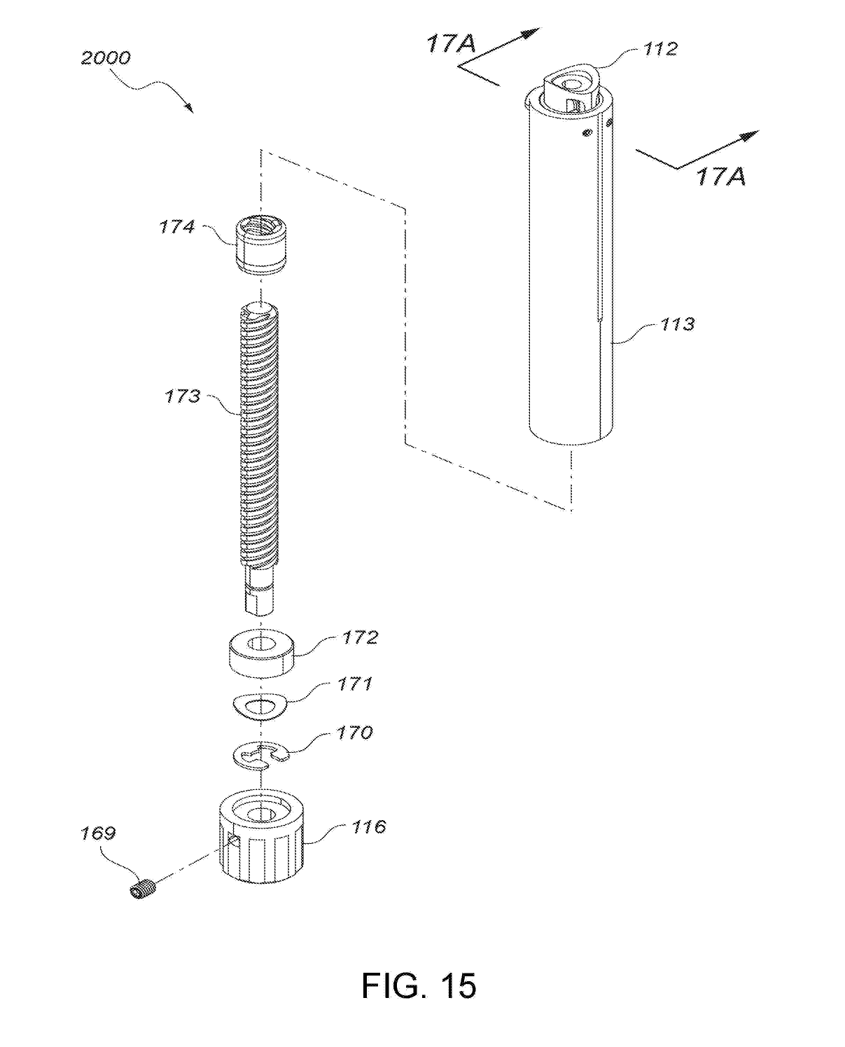

The purpose of the positioning section is to allow for stable positioning of an instrument over a desired anatomical location, by positioning a guiding arm to which the instrument is attached. One of skill in the art would readily appreciate that there are many possible components and configurations thereof that could make up a positioning section of the stereotactic apparatus. In certain embodiments the positioning section includes components that allow for telescoping motion, which permits fine adjustment of the position of the instrument attached to the guiding arm. In some embodiments, a positioning arm is used. In various embodiments, the positioning arm includes two or more nested elements that are operably connected to one another as well as an input component (e.g., a dial) in a manner that allows for telescoping motion. In a non-limiting example, the telescoping motion is accomplished by the components depicted in FIGS. 15-17. The interaction between and operation of the components of FIGS. 15-17 are thoroughly described in the examples section.

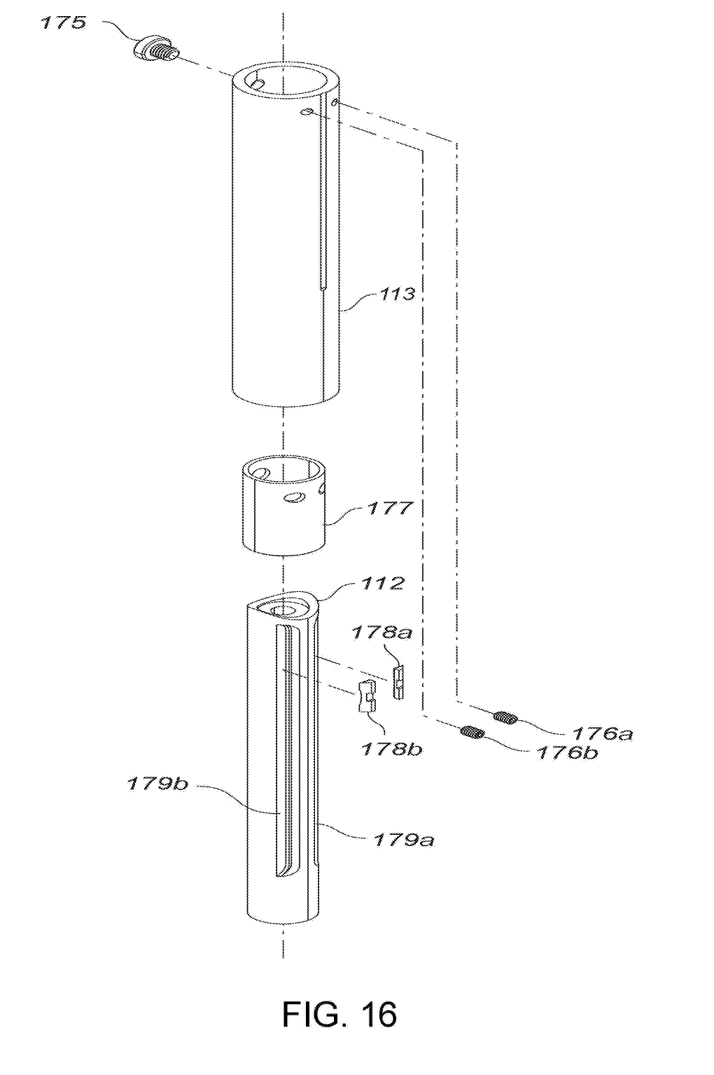

One of skill in the art would readily appreciate that there are numerous possible ways of stabilizing and controlling the telescoping motion of the positioning arm. Merely by way of non-limiting example, if a mechanism with a threaded shaft is used, as depicted in FIGS. 15-17, the number of threadings on the shaft and the pitch of the threadings can be used to dictate the degree to which the positioning arm telescopes in response to associated input (e.g. rotation of a dial). In certain embodiments, the positioning arm is stabilized through the use of components that limit its range of motion in all but the axis along which it is advanced or retracted. Merely by way of non-limiting example, FIG. 16 shows the configuration of guiding set screws 176a and 176b and supporting elements 178a and 178b is used to apply pressure on L-shaped tracks 179a and 179b of inner nested element 112 of positioning arm 2000. FIG. 16 also shows that screw 175 is positioned on the opposite side of set screws 176a and 176b, in order to add to the stability of inner nested component 112, especially while it is being extended or retracted.

One of skill in the art would readily appreciate that there are many possible ways of attaching the positioning arm to the guiding arm. As shown in FIG. 3, one way positioning arm 2000 can be connected to guiding arm 1000 is through the use of screw 133 that traverses the short axis of guiding arm 1000 and connects to grooved receiving socket 134.

### Connecting Section

The long axis of the connecting section of the stereotactic apparatus can be configured to be perpendicular to the long axis of the securing section and the positioning section. In some embodiments, the connecting section, like the positioning section, is a telescoping arm. In some embodiments, the telescoping connecting arm can be stabilized and controlled by any of the aforementioned components associated with the positioning section. Merely by way of non-limiting example, telescoping of the connecting arm can be accomplished through the use of the components shown in FIGS. 12-14, the interaction between which and function of which are thoroughly described in the examples section.

### Guiding Section

The guiding section can be configured to allow for the attachment of one or more instruments that can be extended into and retracted from a subject's body. In some embodiments, the guiding section includes a guiding arm. There are many possible ways by which an instrument can be attached to a guiding arm. One of skill in the art would readily appreciate that the possible components that could be used to attach an instrument to a guiding arm would vary depending upon the dimensions and nature of the instrument to be attached. Merely by way of non-limiting examples, attachment of various instruments to the guiding arm can be accomplished by using one or more straps, clamps, clasps, magnets, and combinations thereof.

Examples of instruments that could be attached to the guiding arm include, but are in no way limited to a cannula (including the floating cannula system described herein), a biopsy needle, a needle, a tube, a cauterization device, a laser, a drill, an endoscope, a guidewire, a fiberoptic device, an electrode, a saw, an ultrasonic device, a spectroscopic device, a camera, an electrical sensor, a thermal sensor, a catheter, a draining tube, an imaging device (such as any of those listed and/or described herein) and the like. In certain embodiments, the instrument guided by the inventive apparatuses described herein includes a guide needle and an injection needle configured to be concentrically housed therein. In some embodiments, the concentric arrangement of the guide needle and the injection needle allows the injection needle to be advanced through the guide needle, once the guide needle is properly positioned in a subject during a medical procedure, so that the injection needle can deliver a payload of biological or chemical material to an appropriate site in the subject. In some embodiments, the instrument guided and/or stabilized by the inventive apparatus is the spinal multi-segmental cell and drug delivery device described in U.S. patent application Ser. No. 12/598,667, which is incorporated by reference herein in its entirety as though fully set forth.

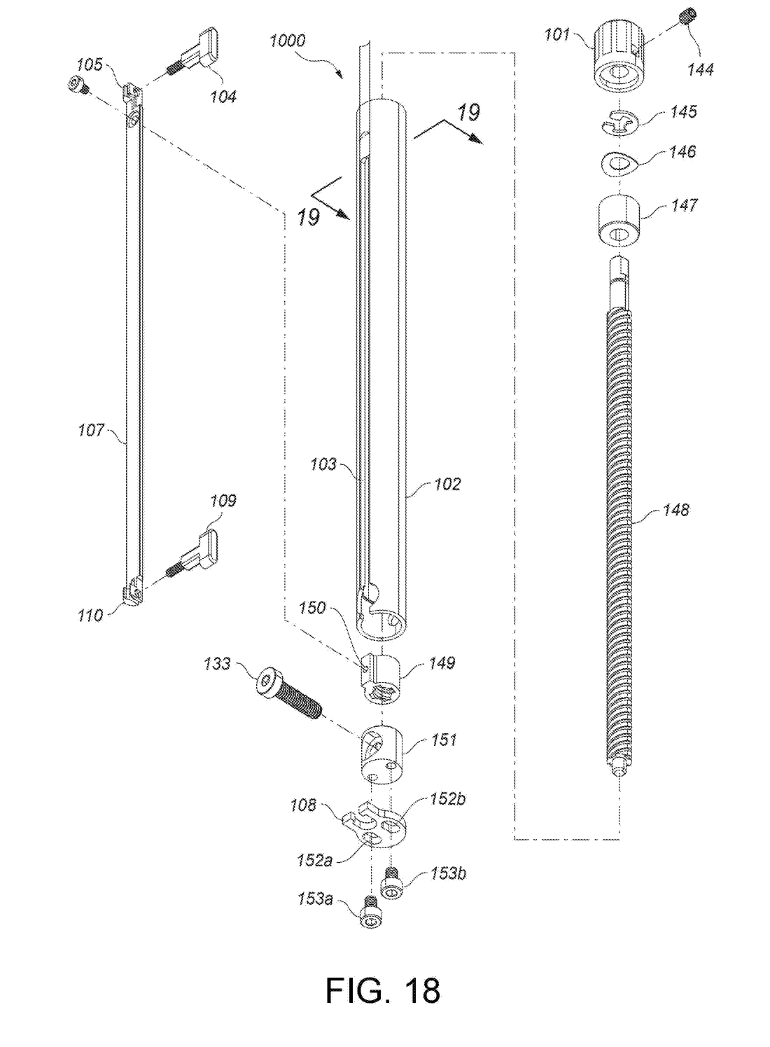

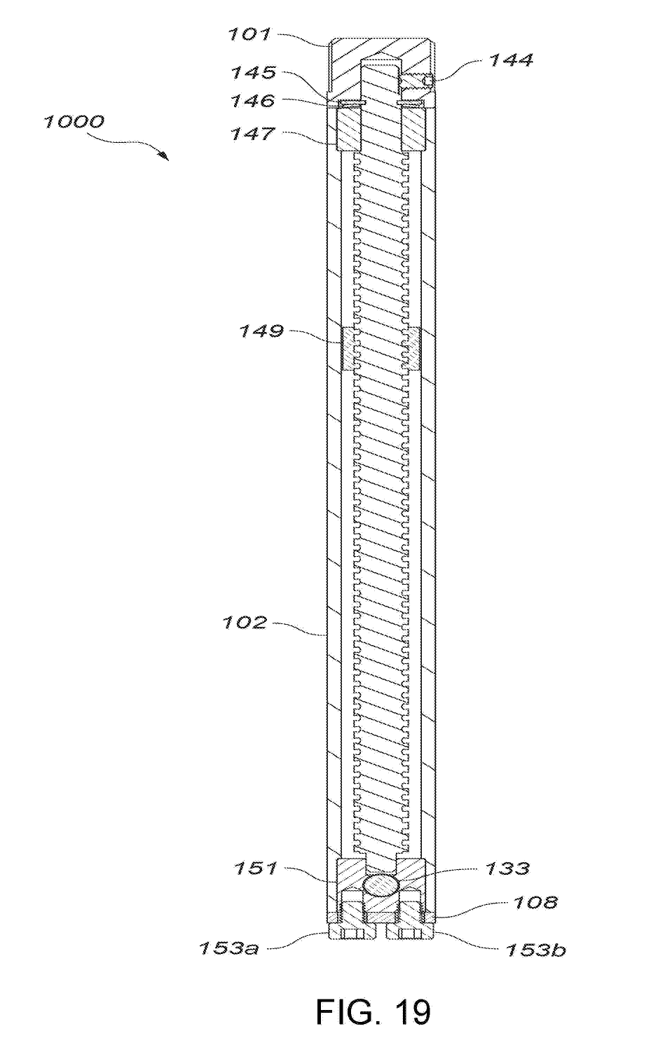

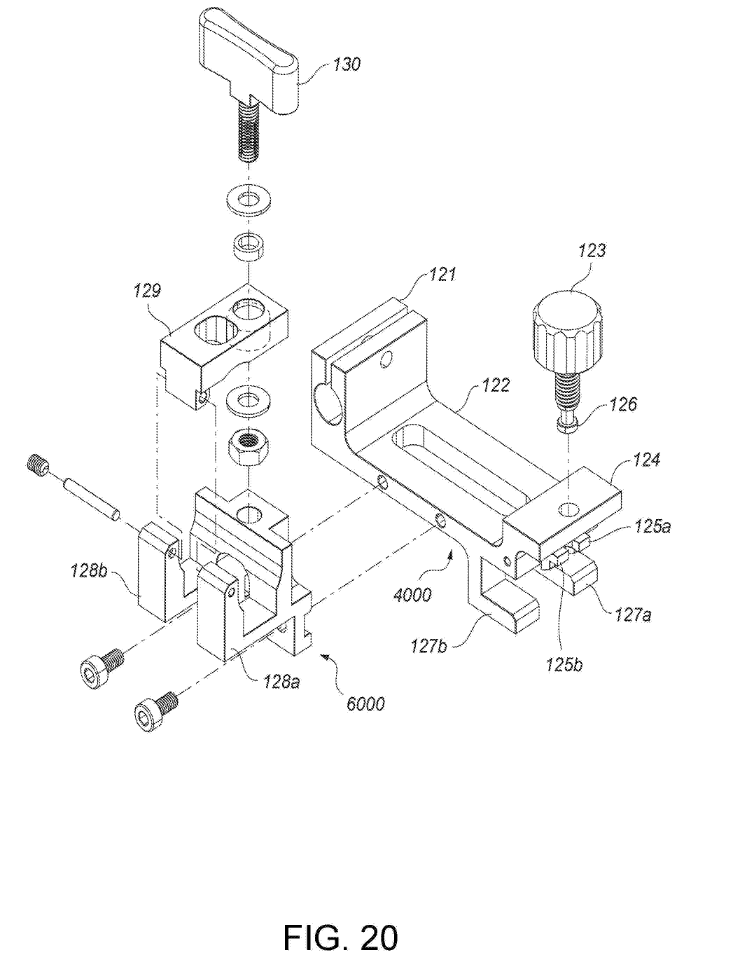

One of skill the art would also readily appreciate that there are numerous possible ways by which the apparatus can be configured to allow for an instrument to be extended into and retract from a subject while connected to the guiding arm. FIG. 18 depicts one non-limiting example of a mechanism that can be used for that purpose. The association between the components shown in FIG. 18 and the function of those components are thoroughly described in the examples section.

### Orientation of Individual Sections

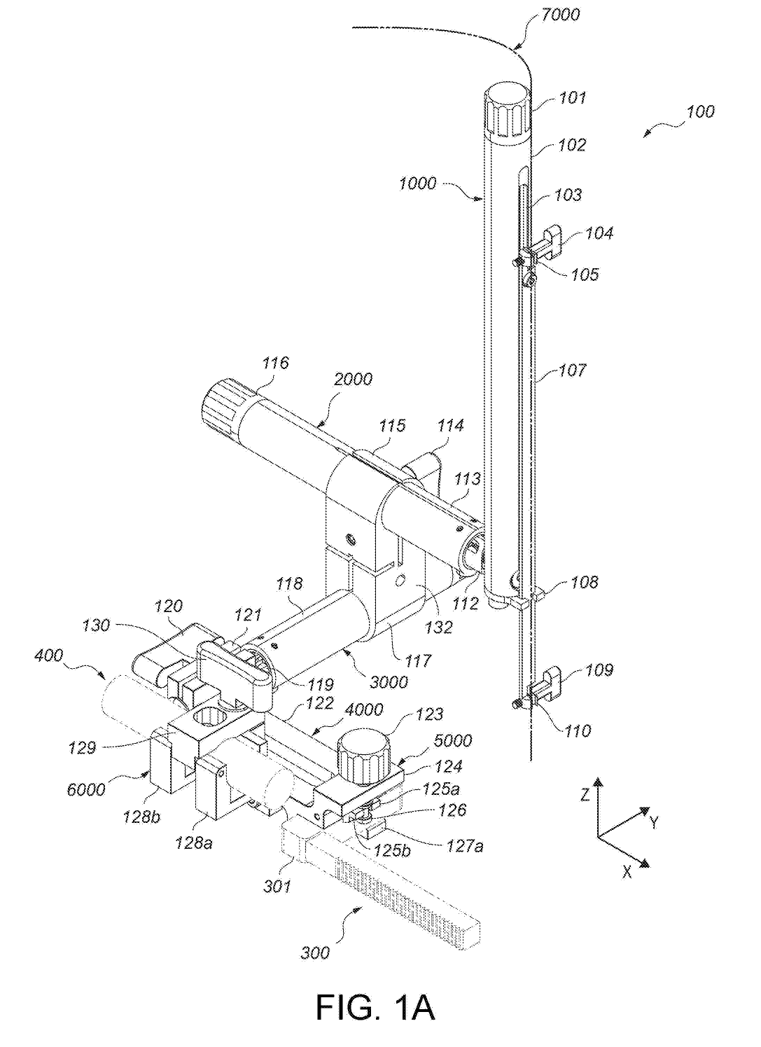

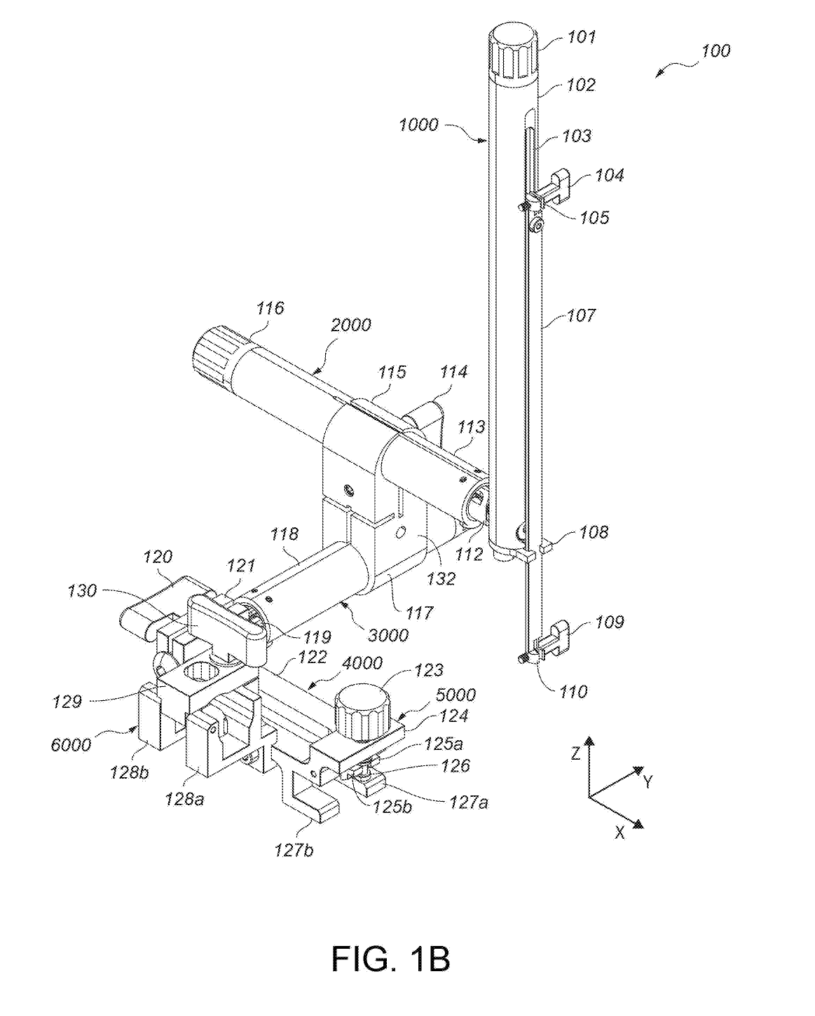

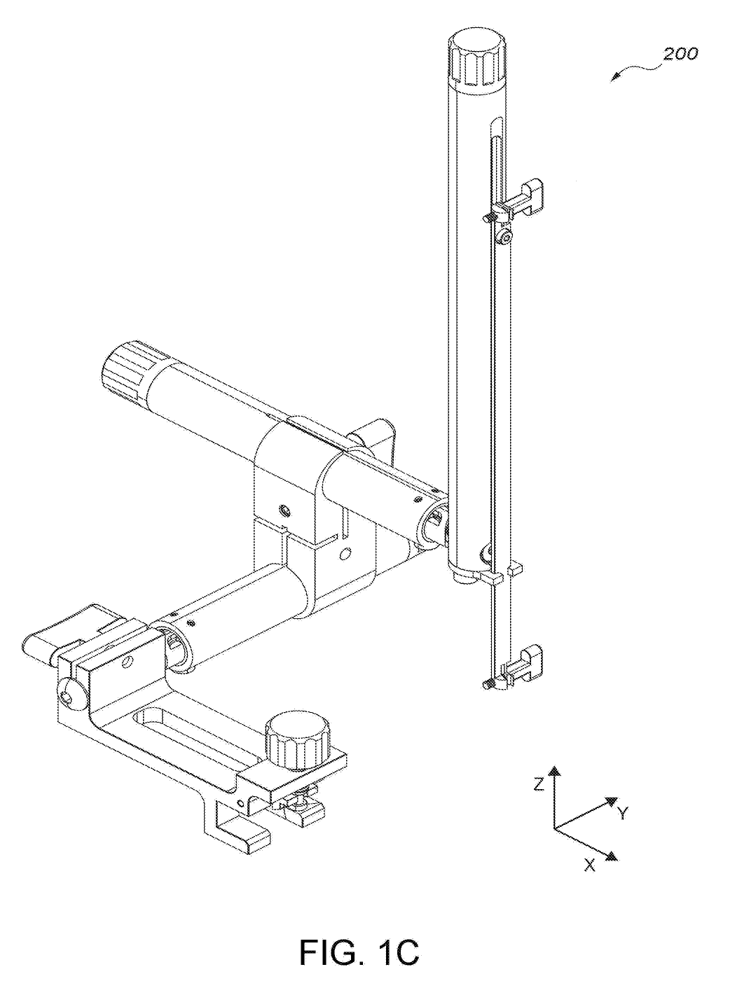

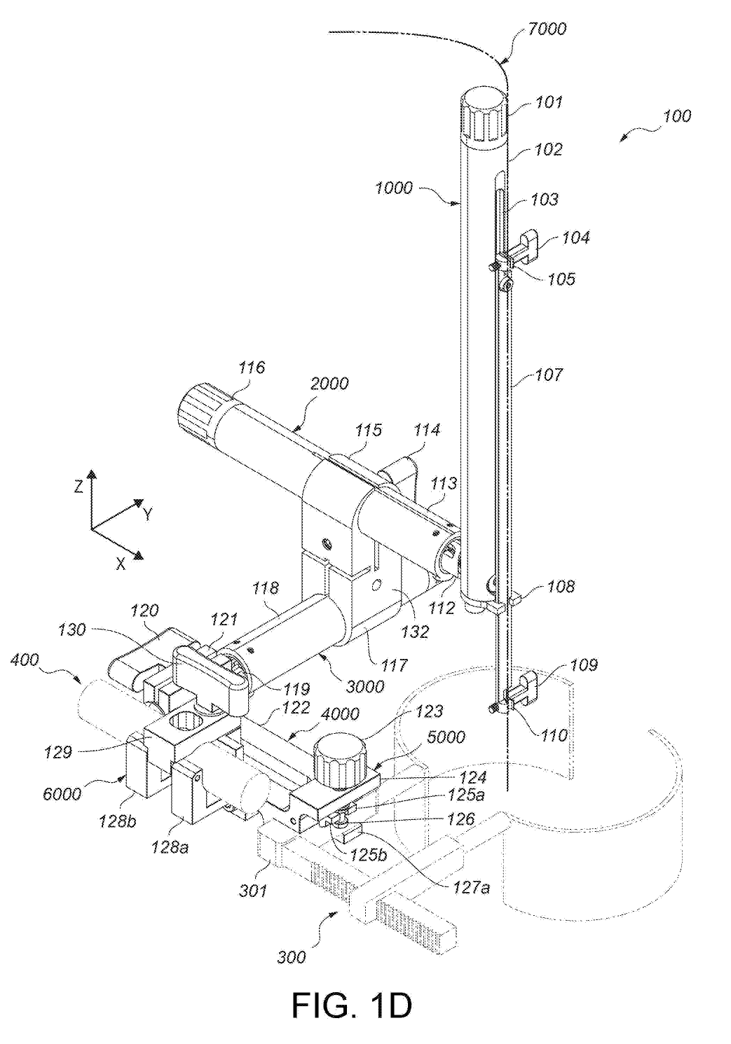

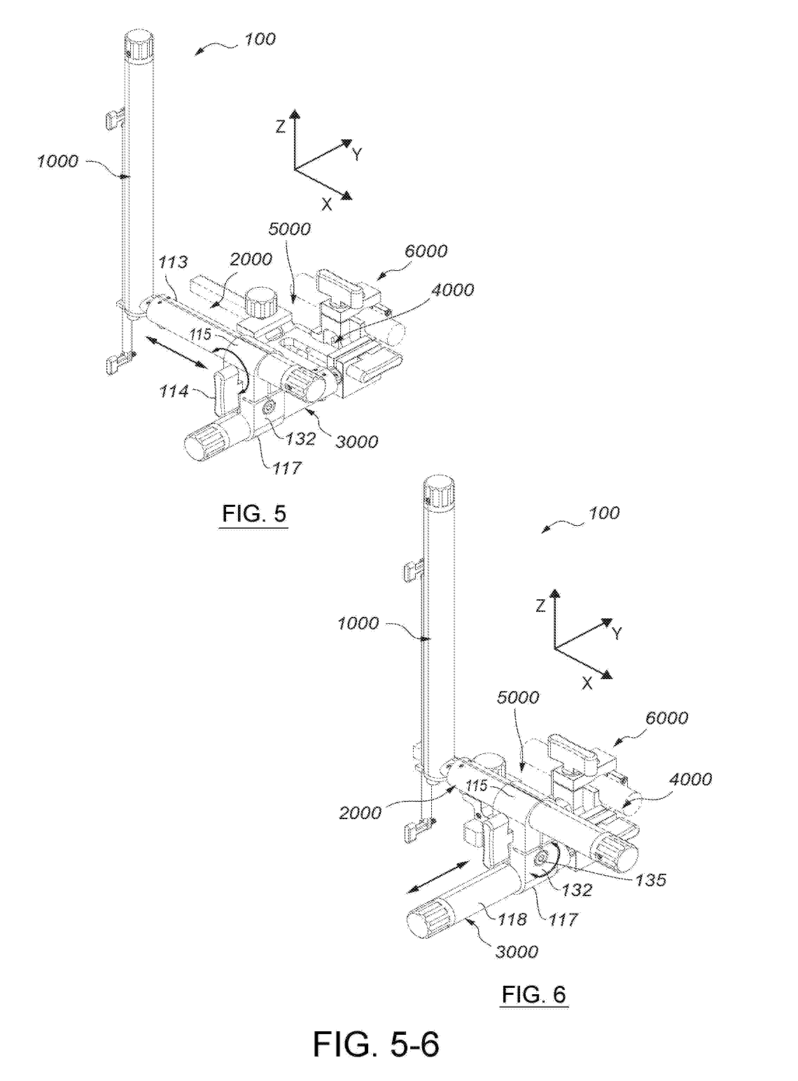

The securing section, connecting section, positioning section and guiding section can be connected to one another by any of a variety of ways depending upon the desired range of motion of each section. In some embodiments, a perpendicular orientation of the positioning arm and connecting arm, relative to one another, is established through the use of a component with perpendicularly situated clamping collars. In an embodiment, cross clamp 132 (depicted in FIG. 1A) can be used. As shown in FIG. 5, when cross clamp 132 is used to secure positioning arm 2000, knob 114 can be rotated to loosen collar 115, thereby allowing for adjustment of the position of positioning arm 2000 along the x-axis. As shown in FIG. 8, loosening of collar 115 by rotating knob 114 also allows for rotation of positioning arm 2000 along the x-axis, which translates into motion of guiding arm 1000 along the y-z plane.

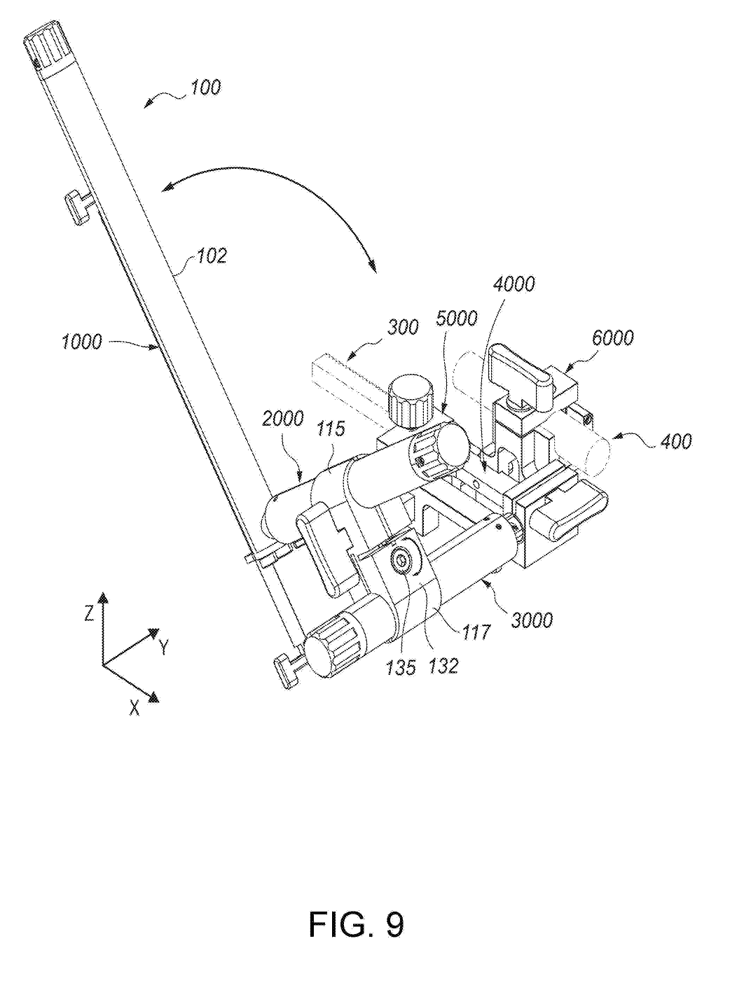

As shown in FIG. 6, when cross clamp 132 is used to secure connecting arm 3000, rotation of screw 135 loosens lower collar 117, which allows for adjustment of the position of positioning arm 2000 along the y-axis. As shown in FIG. 9, loosening collar 117 also allows for rotation of cross clamp 132 along the y-axis, which in turn translates into motion of guiding arm 1000 along the x-z plane.

### Additional Features

The main sections of the stereotactic apparatuses described above can be configured to allow for incorporating additional features on the apparatuses. For example, the stereotactic apparatus can include clamps (or any other means of attachment described herein) situated on one or more of the main sections of the apparatus (i.e. guiding section, positioning section, connecting section, and attaching section) for attaching additional useful instruments or devices.

In certain embodiments, the stereotactic apparatus includes a side clamp attached to the securing section, which allows for attaching a useful instrument or device. For example, as demonstrated in FIG. 3, side clamp 6000 can be used to hold cylindrical device 400. The components of side clamp 6000 are clearly shown in FIG. 22, and thoroughly described in the examples section. One of skill in the art would readily appreciate that a side clamp such as side clamp 6000 can be used to attach any of a number of devices with appropriate dimensions to the stereotactic apparatus.

Devices that can be attached to the stereotactic apparatuses described herein can include, but are in no way limited to, a pump (such as the pump of the syringe pump system described herein), a reservoir for receiving a substance removed from a subject's body, a small motor, a control panel, an imaging device or portion thereof (including any appropriately sized imaging device described herein) and the like. In some embodiments, the device attached is a fiber optic camera that can be positioned to view an opening in a patient's body in which a tissue retractor is engaged. In some embodiments, a reservoir attached to the apparatus can be configured to hold any of a variety of useful substances, including but in no way limited to cells, gasses, liquids, medications, contrast agents, radioactive materials, combinations thereof, and the like.

An additional category of devices that could be attached to one or more sections of the inventive apparatuses described herein is a light source. In various embodiments, the inventive apparatuses may include one or more light sources configured to project light onto a region of interest on or in a subject's body during a medical procedure. In some embodiments, one or more of the light sources is attached to the guiding arm. In some embodiments, the light source is a laser. In some embodiments, the light source is a relatively high energy laser that can be used for cauterizing or cutting. In some embodiments, the light source is a relatively low energy laser that can be used for visually targeting a region on or in a subject's body for incision or other medical intervention. In other embodiments, the light source provides relatively low energy light for aiding in visualizing a region of interest. In still other embodiments, the light source provides light of a wavelength that causes fluorescence of a fluorophore. In various embodiments, the fluorophore is introduced into a subject's body directly, present in cells residing in a subject's body, or naturally occurring. Merely by way of non-limiting examples, the wavelength of the light projected by the light source can be in the visible, IR, or UV range.

Another category of devices that can be incorporated onto the stereotactic apparatuses described herein is an imaging modality. In some embodiments, the imaging modality is attached to the guiding arm. However, one of skill in the art would recognize that all or a portion of an imaging modality (or any other device described herein, or similar thereto) of an appropriate size could be attached to any arm of the apparatuses described herein, by any form of attachment described herein. In some embodiments, the imaging modality includes a device used to perform MRI, CT, or ultrasound imaging. In some embodiments, an endoscope is attached to the guiding arm. In some embodiments, one or more components of a microscope or other magnifying instrument are attached to the guiding arm. One of skill in the art would readily appreciate that any of a number of other useful instruments of a size suitable for attaching to the guiding arm could be used in conjunction with the inventive apparatuses described herein, and attached thereto by any means for attachment described herein.

As indicated above, in some embodiments, the apparatus is configured so that the positions of the various sections described above can be manipulated manually. However, one of skill in the art would readily appreciate that the apparatus could also be configured with one or more motors, gears, pulleys, and electronic controls, so that one or more sections of the apparatus could be electronically controlled.

In some embodiments, the apparatuses described herein are made of stainless steel. In some embodiments, the apparatuses are made of titanium, austenitic steel, martensitic steel, brass, carbon fiber, plastic, combinations thereof, and the like. In preferred embodiments, the material or materials used are biocompatible.

In some embodiments, the invention teaches a method that includes using any of the stereotactic apparatuses described herein for the purposes of facilitating one or more of the processes of (1) introducing a substance into a subject, (2) removing a substance from a subject, and (3) manipulating a portion of a subject's body. One of skill in the art would readily appreciate that the device could be used to introduce a substance into and/or remove a substance from any portion of subject's body, including, but in no way limited to an organ, joint (shoulder, hip, knee, etc.), ligament, tendon, muscle, eye, cavity, or any other tissue. In some embodiments, the substances introduced into the subject's body can include but are in no way limited to biological and/or synthetic substances. Biological substances can include, but are in no way limited to stem cells, neural progenitor cells, tissues, blood, hormones, clotting factors, vectors (including but not limited to viral vectors, plasmids and the like), DNA, RNA, proteins, growth factors, inhibitory substances, matrices, combinations thereof, and the like. Synthetic substances that can be introduced into a subject's body can include but are in no way limited to pharmaceutical agents, markers (including but not limited to biomarkers or any other type of marker that could be visualized with or without the use of imaging equipment), implantable medical devices, electrical sensors, electrical stimulators, glue, sutures, chemotherapeutics, radioactive substances, hyperpolarized substances, combinations thereof, and the like.

Substances that can be removed from a subject's body utilizing the inventive stereotactic apparatuses and methods include, but are in no way limited to, any of the above-named substances that can be introduced into a subject, in addition to tissues, organs, cancer cells and pre-cancer cells, bone marrow, fluid, foreign bodies, combinations thereof, and the like.

In some embodiments, the inventive method includes using any of the inventive apparatuses described herein to position any of the instruments described herein such that they can be introduced between the spreading elements of a retractor device described herein and then the adjacent sections of tissue associated therewith. In an embodiment, the inventive method includes using guiding arm 1000 of inventive apparatus 100 to introduce a needle associated with a cannula into any portion of a subject's spinal cord (including the section specifically described in the non-limiting examples herein). A payload of neural progenitor cells is then advanced through the cannula and needle and into the subject's spinal cord.

In some embodiments, the invention teaches a method that includes (1) attaching any apparatus described herein to the arm of a retractor, (2) attaching any instrument described herein to the guiding arm of the apparatus (by any means described above), and (3) advancing the instrument through the separating elements of the retractor and into a subject's body through an incision in the subject's body. FIG. 1D shows a non-limiting example of how the components of an apparatus can be situated to perform this method.

### Floating Cannula Instrument

In some embodiments, a guiding arm of any of the stereotactic devices described herein may be attached to any of the floating cannula systems described herein. The floating cannula system attached to a guiding arm may be utilized to perform precision injections (including injecting any medically useful substance, whether described herein and otherwise). Merely by way of non-limiting example, the cannula system and stereotactic device may be used when injecting a substance into the spinal cord, thus allowing a caregiver to accurately position the cannula and needle in the correct location.

Typically, once a needle is inserted into a subject's tissue, any movement of the subject with respect to the needle may damage the subject's tissue. This is particularly problematic for injections into sensitive areas, such as the spinal cord, as damage to a spinal cord could have severe consequences. For instance, if a stereotactic device lowered a needle into the spine, and the needle did not provide a stopping mechanism, or allow for movement along the longitudinal axis of the needle, a reflex, twitch, or bucking of the patient could cause the needle to penetrate too far, or otherwise change directions and damage or sever spinal cord tissue (e.g. by shearing). This could have catastrophic consequences to the patient.

Therefore, in some embodiments the invention teaches a floating cannula system that can be attached to the guiding arm of a stereotactic device, and allows for movement of the cannula in response to patient movement, once the needle has been inserted into the patient. In some embodiments, the system includes a floating cannula interacting with a base cannula, where the floating cannula may move up and down with respect to the base cannula to accommodate movement of the patient. The base cannula may be attached to a connector attached to a stereotactic device. This provides stability and support derived from the connector's attachment to the stereotactic device. In other embodiments, the base cannula may be attached directly to the stereotactic device. The base cannula may include two or more support tabs, with holes that receive pins attached to the connector. Additionally, the tabs may include thumb pads for easy manipulation and handling of the cannula by a caregiver.

In some embodiments, the support tabs may include sockets for removably connecting to or mounting the support tabs onto pins that are supported by the connector. This will allow the tabs to hold the base cannula in place while allowing rotation about the pins. In some embodiments, the connector may include a locking member. In some embodiments, the support tabs may be rotated into recesses or spaces in the connector, and then the locking member may be moved to block the support tabs from rotating back out. Accordingly, this system will fix the support tabs and attached floating cannula system in place while the locking tab blocks one or more of the support tabs from coming out of the recess(es). In some embodiments, more than one locking tab may be utilized to block the support tabs.

In some embodiments, the locking member is a physical restraint that creates an interference fit by rotating a handle that blocks the support tabs from rotating out of place. The locking handle may be rotated into place once the tabs are mounted on the pins, and then rotated into one or more slots on the connector. Accordingly, the base cannula, in some embodiments, may be rigidly attached to the stereotactic device through the connector. In other embodiments, the base cannula may attach directly to the guiding arm or other positioning section of a stereotactic device through tabs. In other embodiments, the stereotactic device may include pressure cuffs that attach directly to the round tube of the cannula. Ultimately, a variety of methods/devices may be utilized for attaching a base cannula to a stereotactic device, including one or more of any suitable type of attachment mechanism described herein.

A floating cannula may extend down from the base cannula that is supported by the stereotactic device. In some embodiments, the floating cannula will fit inside the lumen of the base cannula. In other embodiments, the base cannula will fit inside the lumen of the floating cannula. In both embodiments, the concentric fit allows the base cannula to contact the floating cannula while allowing the floating cannula to slide freely along a longitudinal axis of the base cannula and with respect to the base cannula. In some embodiments, the fit between the floating cannula and the base cannula will prevent the floating cannula from moving substantially in other directions, aside from along the longitudinal axis. In other embodiments, the floating cannula and base cannula may be connected to the stereotactic device through a hinged mechanism that allows for motion in a direction perpendicular to the longitudinal axis of the cannulas, in order to accommodate patient movement after the needle is placed.

In some embodiments, the floating cannula will run along the inside lumen of the base cannula and protrude on both sides of the base cannula. Additionally, the floating cannula may include stoppers situated beyond each of the ends of the base cannula on the portions of the floating cannula that protrude therefrom. Accordingly, a top or proximal stopper may be fixed to the top or proximal end of the floating cannula to prevent the floating cannula from falling down and out of the base cannula. Additionally, a lower stopper may be fixed to the distal end of the floating cannula to allow the needle at the bottom to be inserted into the tissue based on resistance from the base cannula pushing on the lower stopper. Otherwise, without the bottom stopper, the floating cannula would not provide significant pressure for insertion of the needle into the anatomical target (e.g. spinal cord) on the subject, when the base cannula is lowered toward the subject.

In some embodiments, the floating cannula will contain a tissue stopper that is attached to the needle. The tissue stopper may be any suitably shaped piece of material secured to the needle that will limit the depth of an injection when the tissue stopper makes contact with the tissue at the injections site. The tissue stopper may be positioned at any point along the needle, depending on the depth of injection required for a particular procedure. The tissue stopper may be any of a number of shapes, including but in no way limited to flat, wedge-shaped, ball-shaped, and cup-shaped. Any suitable shape which provides a mechanical means to limit how far a needle injects into a tissue site (e.g. the spinal cord) is within the scope of the invention.

In some embodiments, the floating cannula will fit inside the base cannula, and thus the stoppers may be positioned on the floating cannula, so that they contact the proximal and distal rims of the base cannula and prevent the floating cannula from moving past certain points with respect to the base cannula. In other embodiments, the floating cannula may fit on the outside of the base cannula (the base cannula would run at least partially inside the lumen of the floating cannula) and may have internal and/or external stoppers. In some embodiments, there will also be space for attaching the base cannula to the stereotactic device through the floating cannula. In some embodiments, the floating cannula will fit inside the base cannula and protrude on both sides of the base cannula. In other embodiments, the base cannula will fit inside the lumen of the floating cannula, but the floating cannula will only cover a distal portion of the base cannula. In this embodiment, other stoppers or movement restriction systems may be utilized to limit the travel of the floating cannula with respect to the base cannula. For example, the base cannula may include a slot, along which a tab connected to the inside lumen of the floating cannula, would ride. The tab may contact another tab on the inside of the lumen of the base cannula that is configured to contact the tab from the floating cannula.

In some embodiments, a delivery tube connected to a liquid reservoir will run the entire length of the system and connect to the needle of the cannula. In some embodiments, the liquid reservoir will be connected to a liquid pump. Accordingly, in some embodiments there may be an external (or internal) pump and reservoir that contain a therapeutic or other injectable substance for injecting into a tissue site (or other location in a subject).

In some embodiments, the invention includes a procedure for injecting a substance into a subject using a floating cannula system and stereotactic device described herein. This procedure may include attaching the floating cannula system to the guiding arm of a stereotactic device (through any mechanism/means described herein). Then, the cannula will be advanced towards an injection site by advancing the guiding arm of the stereotactic device. In some embodiments, the needle will contact the injection site (e.g. the spinal cord) and pressure will push the floating cannula proximally into the lumen of the base cannula. The floating cannula will continue to move proximally until the stopper on the bottom portion of the floating cannula contacts the distal end of the base cannula. Then, the distal end of the base cannula will apply pressure to the stopper, which will be transferred to the floating cannula. Thereafter, the pressure will push the needle into the injection site. In some embodiments, the needle will be inserted until the tissue stopper makes contact with the tissue at the injection site (e.g. the spinal cord). Once the needle is inserted into the tissue, the friction from the tissue on the needle and potentially the negative pressure from the injection site on the needle will hold the needle in place such that it is situated in the direction of the longitudinal axis of the cannulas.

In some embodiments, once the needle is fully inserted into the injection site (as limited by the tissue stopper), the guiding arm of the stereotactic device may be retracted from the injection site, so that the base cannula also moves away from the injection site, and upward with respect to the floating cannula and the needle. This will create space between the distal stopper of the floating cannula and the distal end of the base cannula, which will allow movement of the floating cannula along the longitudinal axis of the cannulas. This freedom of movement will accommodate movement of the subject in whom the needle is inserted (e.g. movement from respiration, heartbeat, bucking, and the like). Accommodation of movement is particularly important in procedures requiring injecting into delicate areas (e.g. the spinal cord), as a sudden force along the longitudinal axis of the needle has the potential to cause the needle to puncture further into the subject and cause considerable damage, depending on the local organs or other anatomical structures in the needle's path.

### Syringe Pump

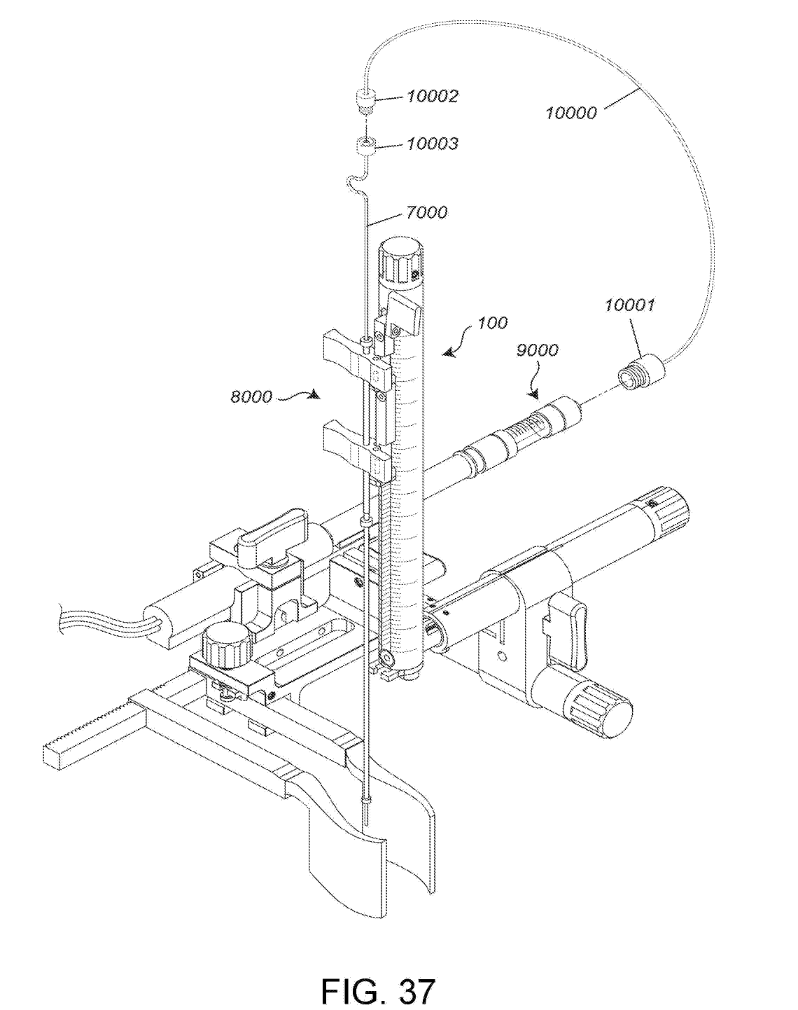

In various embodiments, the invention teaches a syringe pump system that can be used to facilitate the precision injections described herein above, as well as for other purposes. In certain embodiments, the syringe pump system may be configured to attach to a stereotactic device, including any of the stereotactic devices described herein. In some embodiments, the syringe pump system is configured to be secured by the side clamp of a stereotactic device described herein. Although the figures (for example FIG. 37) depict a syringe pump system oriented in one direction relative to the stereotactic device, the syringe pump systems described herein can also be oriented in the opposite direction relative to the stereotactic device. In addition, the syringe pump systems described herein may be configured to interact with and attach to (permanently or removably) any of the cannulas and cannula systems described herein, including the floating cannula systems described herein, whether for the purpose of facilitating the injection of a therapeutic substance into a subject (as described herein), or otherwise.

The syringe pump systems described herein all include the following central components: (a) a carpule with an interior chamber configured to hold a quantity of a therapeutic substance or other medically useful substance, (b) a plunger configured to interact with the interior chamber of the carpule and advance therein in order to expel a therapeutic substance (or other medically useful substance) therefrom, and (c) a motor for imparting a drive force (either directly, or indirectly—i.e. through one or more drive shafts) to the plunger. In some embodiments, the syringe pump systems described herein may be utilized to deliver therapeutic agents, such as stem cells, pain medications, chemotherapeutic agents and/or other medications (along with any other medically useful substance or combination of substances described elsewhere herein), safely, by regulating fluid dynamics and monitoring flow pressure during injection. In preferred embodiments, the size of the syringe pump system may be such that it does not significantly encumber the surgical space for the procedure in which it is utilized. As such, the syringe pump system may be configured to be a small hand-held device and/or a stand-alone pump that may be utilized in surgical procedures that do not require a stereotactic system.

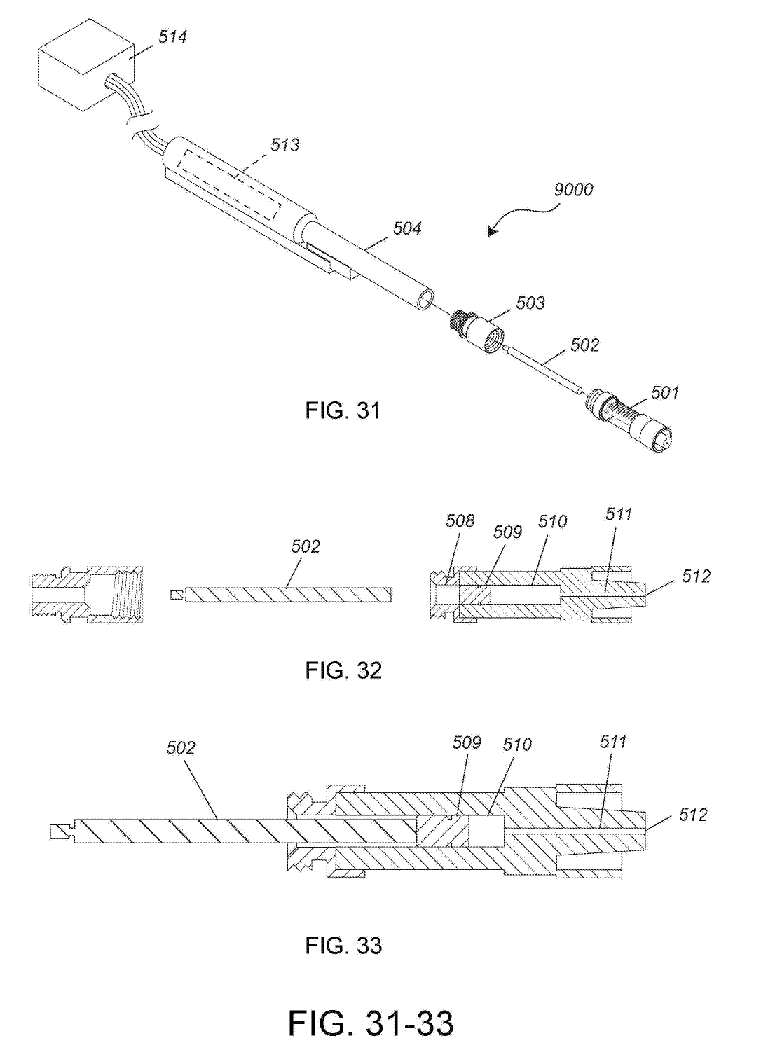

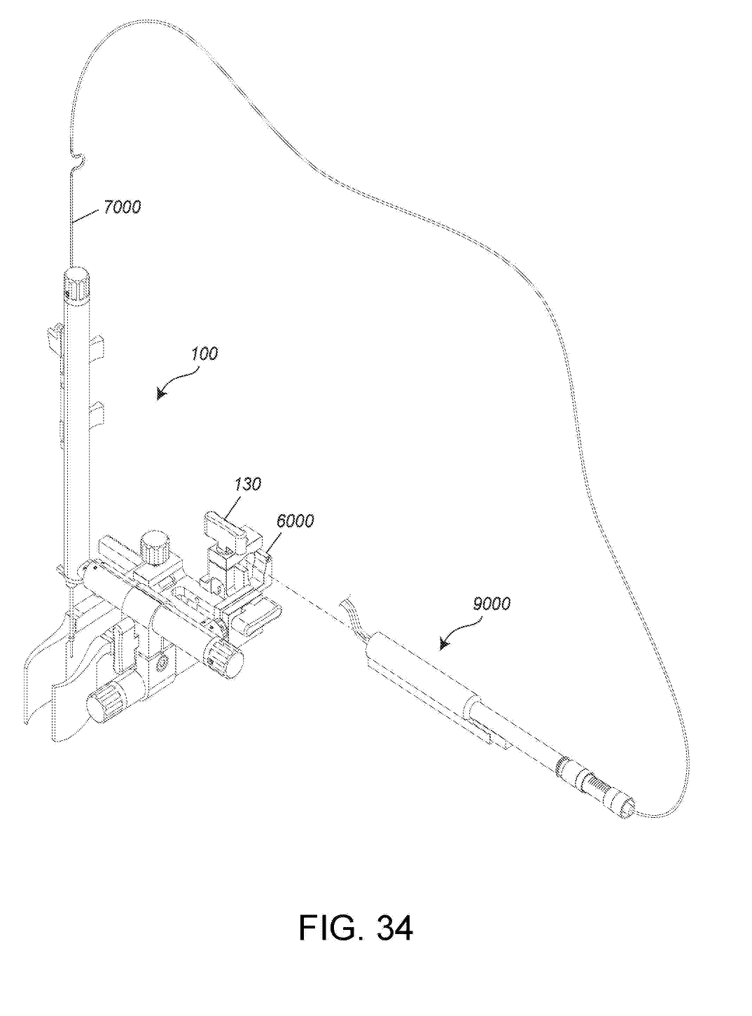

With regard to the carpule component, in certain embodiments the carpule may be configured as a disposable component that is removably coupled to the syringe pump. In some embodiments, the carpule component may contain a therapeutic agent (or other medically useful substance) with a predetermined amount and/or dosage to be injected into a specific anatomical location and/or tissue (e.g. spinal cord, brain, tumor tissue, etc.). A drawing of one embodiment of a syringe pump system including a carpule is shown in FIG. 31.

In certain embodiments, the carpule is made of one or more sterilizable materials (e.g. glass, plastic, metal, etc). In some embodiments, the carpule has an interior chamber with a volume of 50 ul. In some embodiments, the carpule may have an interior chamber with a volume of 100 ul, 250 ul, 500 ul, or more. In various embodiments, the volume of the chamber may be from 20 ul to 10 ml or more. The size of the carpule and volume of its interior chamber may be configured to be appropriate to accommodate a volume and dosage of a therapeutic agent (or other medically useful substance) needed for a particular application/procedure. In certain embodiments, the interior chamber is cylindrical, but other shapes are possible and within the scope of the present invention. In certain embodiments, the carpule is removably coupled (directly or indirectly) to a component including a motor and drive shaft and/or plunger. In some embodiments, the carpule is configured to be prefilled with a therapeutic agent (such as any type of cellular therapeutic composition described herein) prior to use. In some embodiments, the carpule may be removably coupled to the syringe pump, so that it may be sterilized before and/or after use in a medical procedure (e.g. by gamma radiation, EtO, etc.). In some embodiments, the syringe pump system may also include a mechanism to rotate and/or vibrate the carpule component, in order to reduce or avoid settling, clogging and/or clumping of the therapeutic agent (e.g. cells). In some embodiments, the syringe pump system is configured to deliver a therapeutic agent via a microfluidic flow process.

Merely by way of non-limiting examples, the interior chamber of the carpule may be smooth, rigid, and/or contain grooves. In certain embodiments, the carpule may be designed such that it has a cone-shaped interior, in order to facilitate fluid flow out of the carpule. By way of non-limiting examples, the cone-shaped interior may be smooth, rigid, and/or contain grooves. In some embodiments, the carpule may contain markings on the interior or exterior surface. The markings may allow the user to determine how much volume of a substance has been loaded in and/or expelled from the carpule. In some embodiments, the carpule includes a window made of glass, plastic, or another transparent or semi-transparent material that allows the substance in the chamber to be viewed.

In certain embodiments, the carpule includes (a) a first end including an elongated inlet port, (b) a second end including an elongated outlet port, and (c) a chamber disposed between and in fluid communication with the elongated inlet port and the elongated outlet port. In some embodiments, the chamber, elongated inlet port and elongated outlet port are approximately the same size (i.e. diameter). In other embodiments, these sections of the carpule are different sizes.

Turning now to the plunger component, in some embodiments the plunger is elongated and it includes a receiving plunger end, a pushing plunger end, and an elongated plunger body. In some embodiments, the plunger is configured to nest within the first elongated inlet port of the carpule. In certain embodiments, the pushing end of the plunger is configured to form a fluid-tight seal with the interior chamber of the carpule. In some embodiments, the receiving plunger end is configured to receive pressure from a drive shaft attached to the motor, such that the plunger is advanced along the interior chamber of the carpule, thereby expelling liquid contained in the chamber. In other embodiments, the plunger is directly attached to the motor, and configured to advance along the chamber of the carpule in response to input from the motor.

With regard to the motor of the syringe pump system, in certain embodiments the motor (513 in FIG. 31) is contained within a housing (504 in FIG. 31). In some embodiments, the syringe pump is connected to a control box (514 in FIG. 31), which electronically controls the flow of fluid pumped by the syringe pump (rate, duration, volume, etc.). As indicated above, the motor of the syringe pump may be connected to a drive shaft for imparting a drive force on the plunger, thereby causing the plunger to advance along the interior chamber of the carpule and expel a therapeutic or other medically useful substance contained therein.

Although the syringe pump can be connected to a control box (or other controller) via wires (as shown in FIG. 31), a wireless connection to the control box/controller is also within the scope of the present invention, and can be accomplished utilizing any appropriate wireless transmitters and receivers known in the art.

With respect to its power supply, in some embodiments the syringe pump may be battery operated, while in other embodiments the syringe pump may include a power cord to connect to a power source.

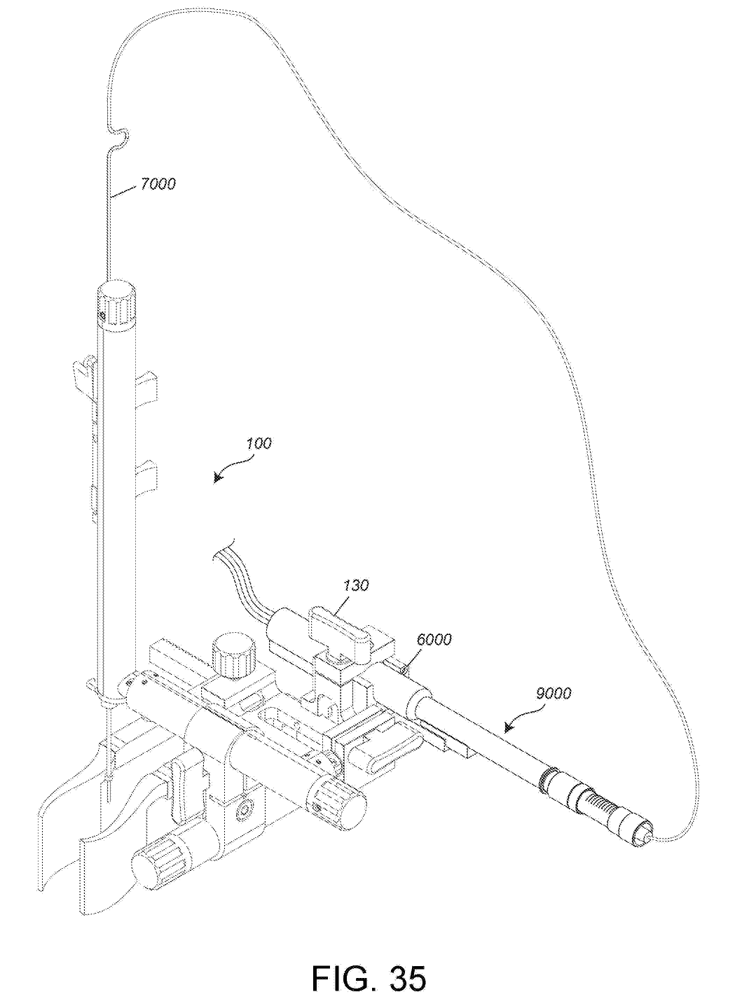

With respect to its shape, in certain embodiments the syringe pump may be substantially cylindrical, such that it may be held in a side clamp of a stereotactic device (as described herein and shown in FIG. 35) and/or easily held by a user. In other embodiments, the syringe pump may be configured to be a different shape, which is useful for a particular application/procedure.

In certain embodiments, the syringe pump system may also include a connector/coupler component. In some embodiments, the connector/coupler contains threading on one end to allow for its attachment to the housing that contains the motor of the syringe pump. For example connector 503 in FIG. 31 has threading that mates with grooves on the inside of housing 504. Although threading can be used to attach the connector/coupler to the housing of the syringe pump, the connector/coupler may also be attached by any mechanism for attachment described herein that is suitable for that purpose. In some embodiments, the connector/coupler is configured (with threading or otherwise) to attach to a syringe pump motor housing with its first end, and a carpule with its second end, thereby facilitating the connection between the syringe pump motor and drive shaft and the carpule and plunger components (as shown in FIG. 31).

In certain embodiments, the syringe pump system may also include a blockage detection device that monitors variations in flow pressure in the interior of the carpule component (e.g through a flow sensor). In some embodiments, the flow rate may be controlled by the speed of the motor and the force exerted (directly or indirectly) onto the plunger.

In various embodiments, the syringe pump system further includes a delivery tube connecting the carpule to a cannula. In certain embodiments, the syringe pump system further includes a cannula described herein. In some embodiments, the cannula connected by the delivery tube to the syringe pump is a floating cannula described herein. In some embodiments, the delivery tube is made of PTFE. In other embodiments, the delivery tube is made of any suitable material known in the art (e.g. any suitable plastic, etc.).

In some embodiments, the syringe pump system includes a flexible and sealable carpule delivery tube. This component may be used in conjunction with the carpule component described above, and can be configured in the manner of the flexible and sealable delivery tube 10000 shown in FIG. 37. In some embodiments, the flexible and sealable carpule delivery tube may attach to the carpule through a coupling component situated on its first end. This attachment may be accomplished, for example, through a threaded coupling component 10001, as shown in FIG. 37. The carpule delivery tube may also be configured to simultaneously attach to a cannula system. Merely by way of example, this can be accomplished by incorporating complimentary Luer lock fittings 10002 and 10003, as shown in FIG. 37. In some embodiments, the carpule delivery tube may include a valve at one or both ends that serves to allow fluid to flow only in the direction of the cannula. In some embodiments, the carpule of the syringe pump system may be pre-loaded with a sterile saline solution, and the carpule delivery tube may be pre-loaded with a solution that includes cells (including any type of cell described herein). Thus, when the syringe pump is activated, the plunger of the syringe pump pushes the saline solution through the end of the carpule, which in turn advances the cells through the carpule delivery tube, then through the cannula system, and finally both the cells and saline flow through a hollow needle at the tip of the cannula and into a target site in a patient into whom the needle has been introduced. In some embodiments, the carpule delivery tube may prevent settling, clogging, or clumping of the therapeutic agent being expelled therefrom (e.g. cells).

In some embodiments, the carpule itself is pre-loaded with a therapeutic liquid substance (e.g. cells), and the therapeutic liquid substance is pumped from the carpule, through a delivery tube, then through a cannula, and finally into a target site in a subject. The delivery tube used for these embodiments can be any delivery tube described herein, and the cannula can likewise be any cannula described herein.

### Imaging System

One of skill in the art would readily appreciate that the imaging systems described herein could be used in conjunction with many types of surgical procedures in which a tissue retractor device is typically used. The inventive imaging systems are especially effective when used in conjunction with the procedures for performing spinal cord injections described herein. In preferred embodiments, the imaging systems of the invention are used while performing injections into the spinal cord of a subject who has been diagnosed with amyotrophic lateral sclerosis (ALS), according to the methods described herein.

Until the development of the present technology, imaging used to determine the proper placement of a cannula needle into the spinal cord was limited to preoperative imaging such as an MRI. Incorporating intraoperative imaging of the spine by using the inventive imaging systems improves the accuracy of injections, because it can help identify/map key landmarks prior to and during the injection process. These landmarks may include, but are in no way limited to, gray matter, white matter, the dorsal horn, the ventral horn, the central canal, and the spinal cord roots.

In some embodiments, the intraoperative imaging components described herein (e.g. radiation source and radiation detector) are configured to be coupled to a retractor device described herein. Once the imaging components are coupled with the retractor device, an arm of the retractor device can be attached to a stereotactic device (as described herein). In certain embodiments, the intraoperative images may be produced dynamically, such that a physician can change coordinates and/or parameters of the stereotactic device during the procedure, which is especially useful when an automated stereotactic device is utilized.

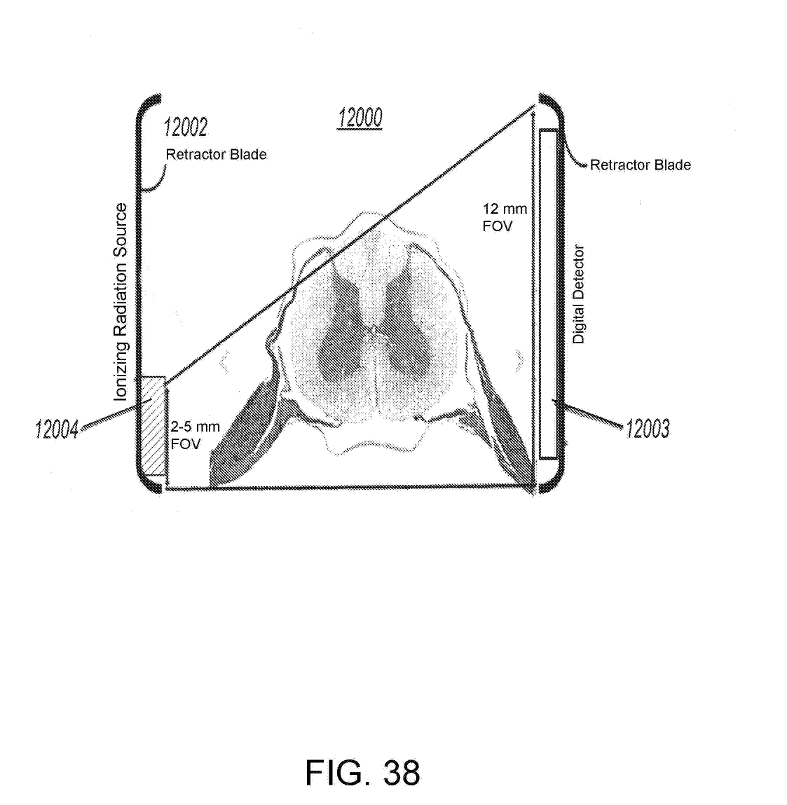

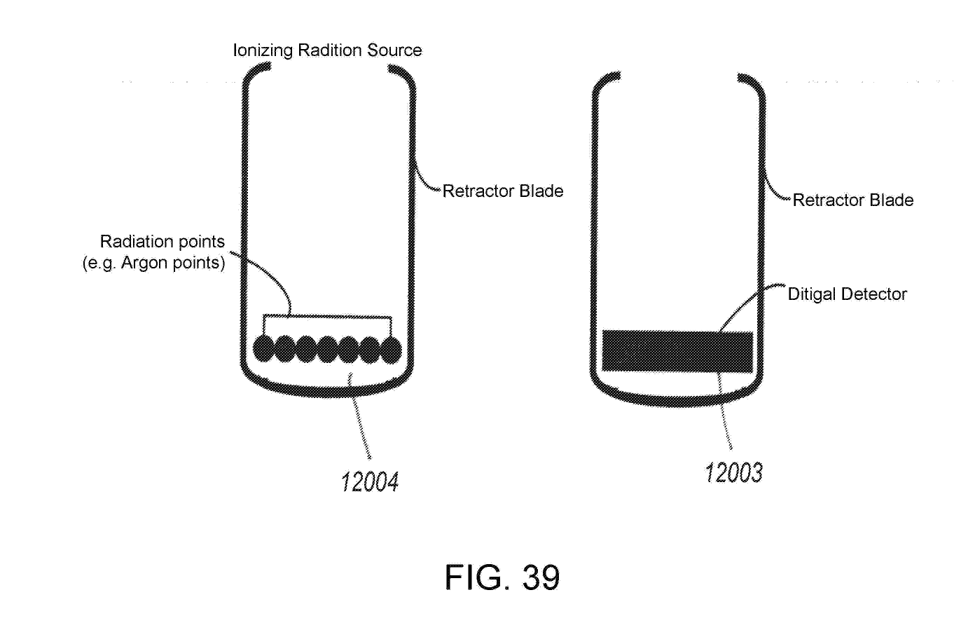

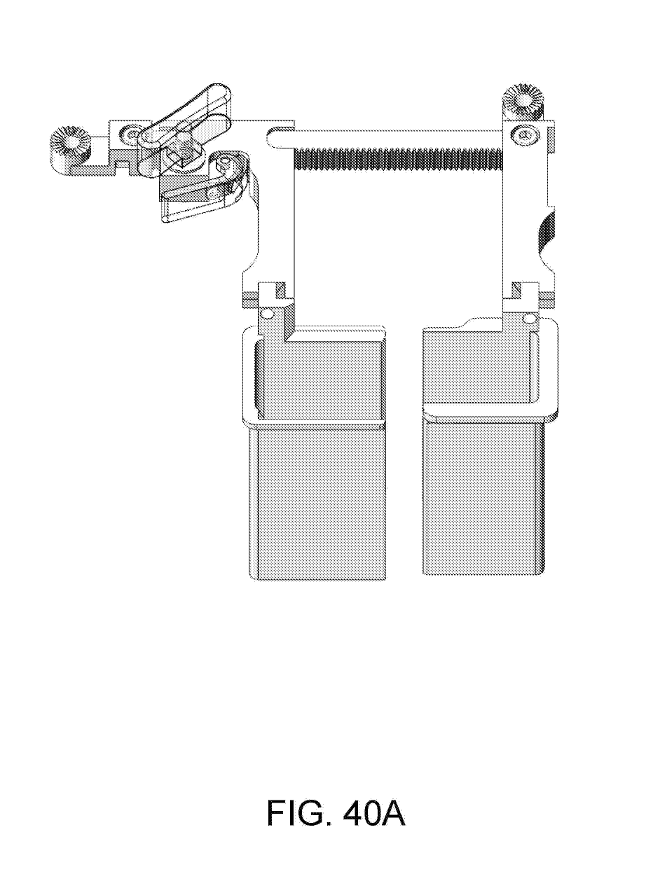

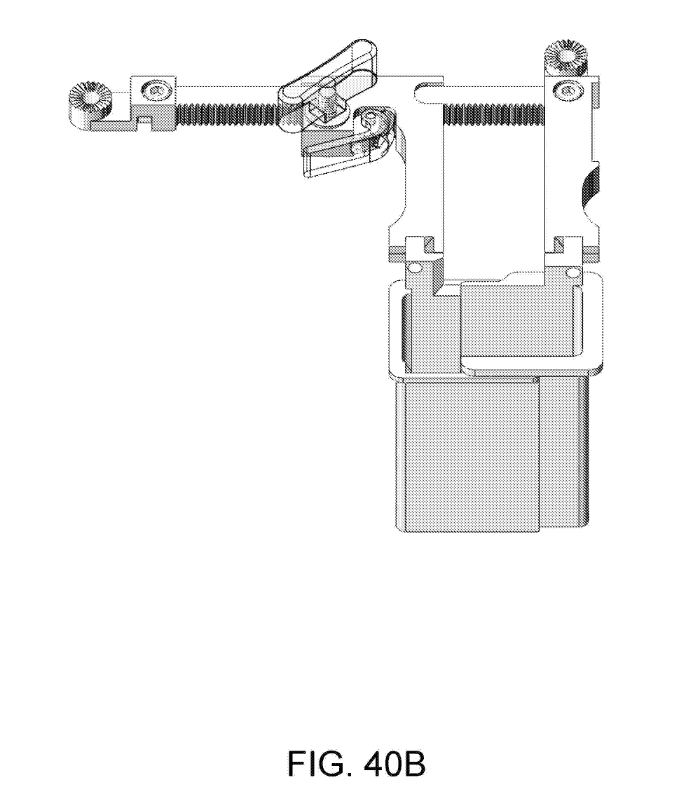

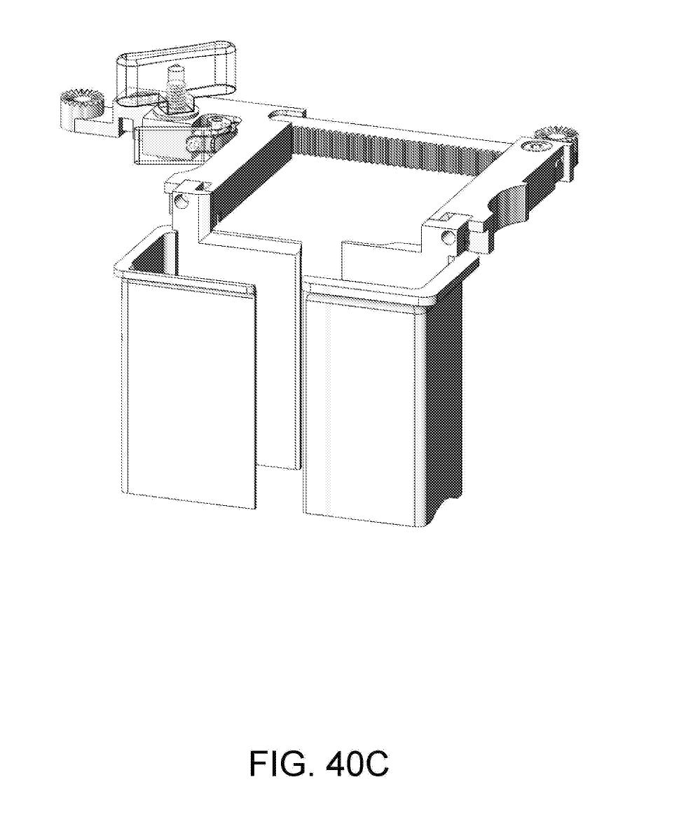

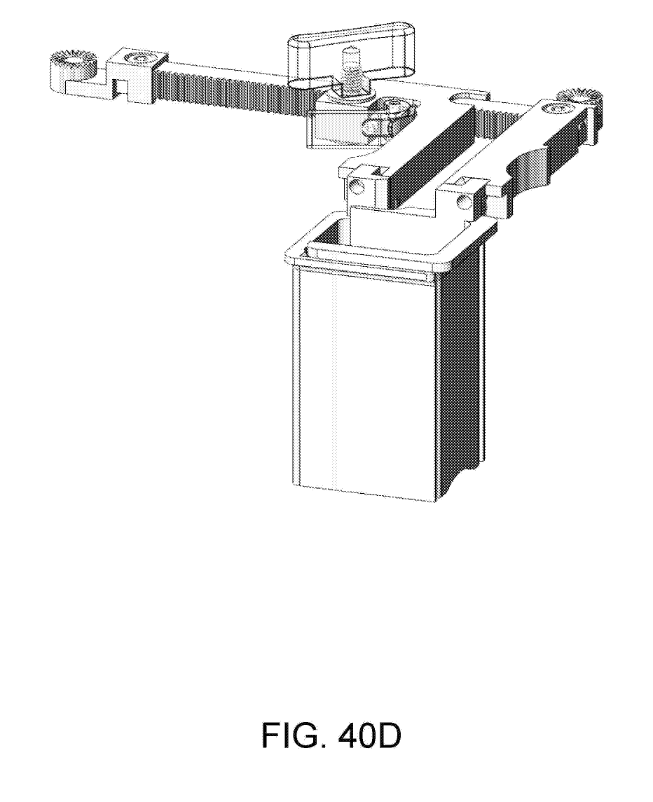

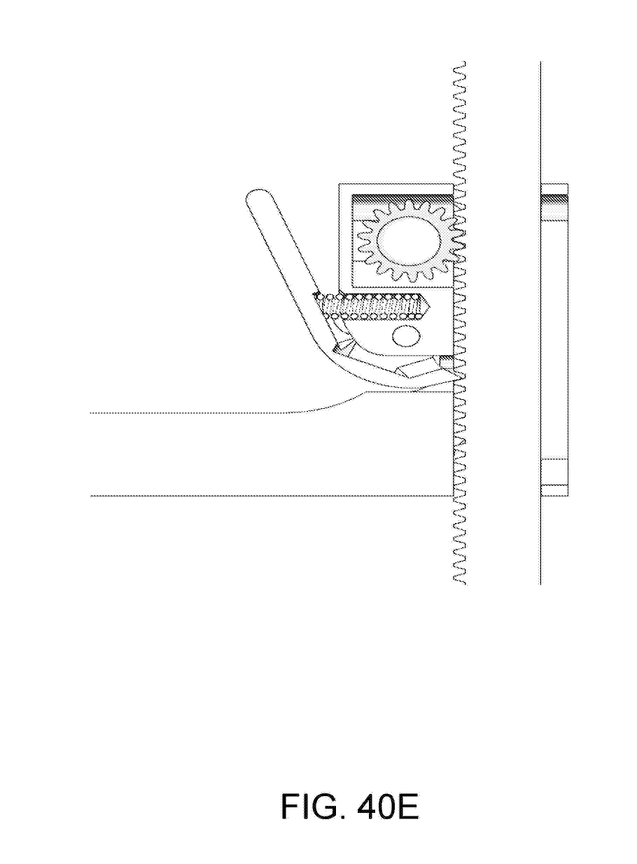

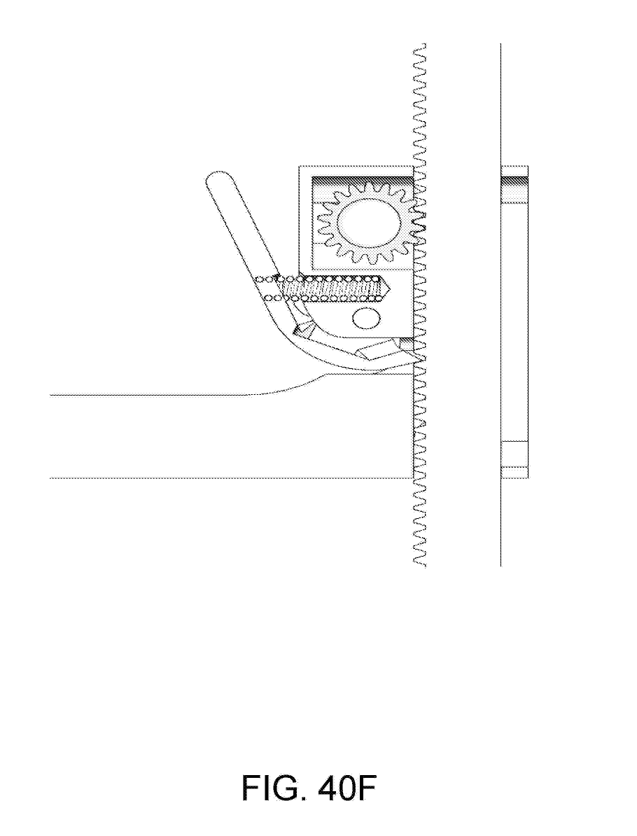

In various embodiments, the intraoperative imaging system may include a retractor device with two or more retractor blades (e.g. any type of tissue retractor described herein), an ionizing radiation source and a digital radiation detector, wherein the ionizing radiation source and digital radiation detector are each attached to opposing blades of the retractor device. Merely by way of example, the ionizing radiation source and radiation detector may be configured and oriented as shown in FIGS. 38 and 39.

The system may further include wires that connect the radiation source and/or digital radiation detector to a computer capable of one or more of (1) controlling and/or receiving input from the radiation source (e.g. controlling the dose and duration of radiation exposure), (2) controlling and/or receiving input (e.g. data) from the detector, (3) processing the data generated by the detector, and (4) generating one or more images from the processed data that can be displayed on a video monitor (or other like device) connected (wired or wirelessly) to the computer. In some embodiments, the controlling functions are performed by a separate device from the device that processes the data and generates the images. In other embodiments, both functions are performed by the same computing device. In some embodiments, all or some of the aforementioned connections are wireless.

In some embodiments, an arm of the retractor device to which the radiation source and detector have been coupled is attached to a stereotactic device, in a manner described herein.

Procedurally, a first retractor blade of the imaging system may be positioned on a first side of an anatomical region of interest (e.g. the spinal cord), and a second retractor blade of the imaging system may be positioned on a second side of the anatomical region of interest, such that a section of the anatomical region of interest is situated between the first and second retractor blades, and therefore also between the ionizing radiation source and detector.

In some embodiments, the retractor blades may be positioned on opposite sides of a subject's spinal cord (accessed, for example, using the procedure described in the examples section). A stereotactic device with a cannula attached thereto (e.g. the floating cannula system described herein) may be attached to the arm of a tissue retractor of the imaging system. The stereotactic device may also be attached to a syringe pump system, as described herein. In this way, imaging can be performed with the imaging system prior to, during, or after performing the spinal cord injections by the methods described herein.

In various embodiments, the ionizing radiation source may include radiation collimation points (e.g. argon collimation points) configured to emit radiation at specific points where the injection is taking place, based upon a required field of view (e.g. as situated in FIG. 38). The collimation may be accomplished by any electronic means or device/filter known in the art.

Merely by way of example, the radiation source and/or system (or a substantially similar system) described in U.S. Pat. No. 8,530,854, which is incorporated herein by reference in its entirety as though fully set forth, can be used in conjunction with the inventive device. Furthermore, the radiation source may be any radiation source configured to be an appropriate size for a given procedure. The radiation source may emit X-rays, gamma rays, or energy within any range of wavelengths useful for imaging required in a specific procedure.

With respect to the radiation detector/detecting mechanism, the imaging system may include a scintillator operatively connected to a digital processor of a computing system. In some embodiments, once the radiation detector detects radiation from the radiation source, this information is communicated to the computing system for processing. In some embodiments, the information is communicated wirelessly, while in other embodiments one or more wires are used to connect the detector to the computing system. In some embodiments, the computing system is configured to generate one or more images based upon the processed information/data.

In some embodiments, the radiation source may have dimensions of 2 to 5 mm in length, but the source may be configured with any suitable dimensions appropriate for the particular procedure being performed. In certain embodiments, a single radiation point may have a 1 to 15 mm field of view. In certain embodiments, the field of view is approximately 5 mm.

In some embodiments, the digital detector may have a 1 to 20 mm field of view. In some embodiments, the digital detector may have an approximately 12 mm field of view. When the imaging system is utilized to image the spinal cord (e.g. for any of the procedures described herein), the field of view is sized in order to capture one or more region of interest of the spine or spinal cord, including but not limited to gray matter, white matter, the dorsal horn, the ventral horn, the central canal, and the spinal cord roots.

In some embodiments, the intraoperative imaging system may have from 0.02 to 2 mm pixel spatial resolution. In one embodiment the imaging system may have an approximately 0.7 mm pixel spatial resolution. In some embodiments, when configured to image the spinal cord, the imaging system will have resolution sufficient to allow for distinguishing between gray and white matter of the spinal cord. In some embodiments, the resolution will allow for imaging the cannula needle inserted into the gray matter of the spinal cord (according to the procedures described herein).

As indicated herein, in certain embodiments, the data from the detector will be digitally captured and transferred to a computer for processing. In some embodiments, a supplemental or stand-alone processor may be connected to the detector, in order to preliminarily or completely process the raw data generated therefrom. In other embodiments, there is no preliminary processing accomplished at the site of the detector, and all of the processing is accomplished by a computer located outside of the surgical field. The computer may receive information from the detector through one or more wires connected to the detector, or from a wireless receiver that receives information through a wireless transmitter connected to the detector. The imaging system may be configured to capture one or many images, depending upon its intended use.

In some embodiments, the imaging system is configured to image the spinal cord during the procedures described herein for patients receiving a cellular therapy for ALS. In some embodiments, imaging is performed before each injection. In some embodiments, imaging is performed during each injection. In certain embodiments, imaging is performed after each injection. In some embodiments, imaging is performed in conjunction with the automated components described herein. In certain embodiments, the cannula-mediated injections (including optionally by the floating cannula) can be performed as described herein, with or without the use of the syringe pump mechanism, by using the imaging system to determine where the injection should be placed, and then, once the proper coordinates are determined, utilizing the stereotactic device (as described herein) to perform the injections at the proper location(s). In certain embodiments, contrast dye can be used to enhance the imaging, depending upon the anatomical structure and procedure.

Because the images are digitally captured by the imaging system, relatively low radiation will be used and the user may monitor the images dynamically. In one example, the user may be able to view the images using an image workstation. In another example, the user may be able to view the images using a miniaturized computer and/or monitoring system such as Google glass, or a similar technology.

### EXAMPLES

### Example 1

### Stereotactic Apparatus with Side Clamp

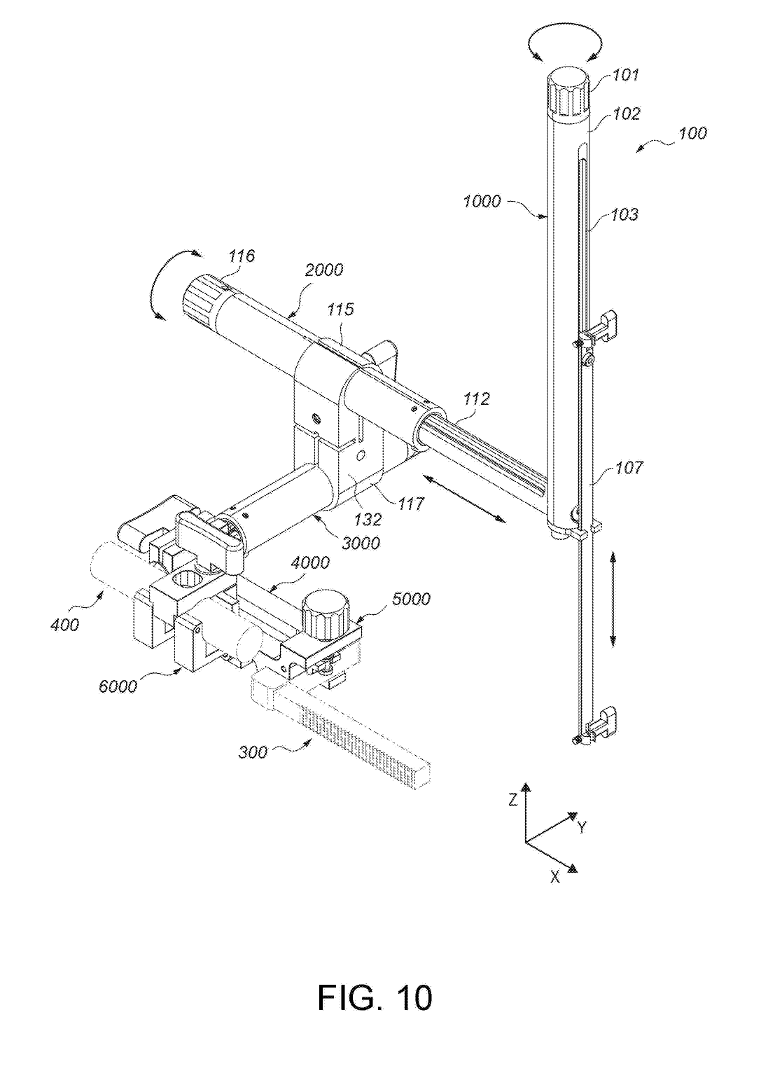

FIG. 1A depicts exemplary stereotactic apparatus 100. Stereotactic apparatus 100 includes guiding arm 1000, which includes an elongated channel 103 situated along its long axis (FIG. 1A). Guiding arm 1000 includes a dial 101 and an elongated cylindrical body 102 (FIG. 1A). Guiding arm 1000 also includes instrument attachment component 107, and clamps 105 and 110 which are tightened and loosened by screws 104 and 109, respectively (FIG. 1A). The guiding arm 1000 further includes instrument attachment component guide 108. FIG. 18 depicts an exploded view of guiding arm 1000, in which the assembly of threaded shaft 148, bushing 147, curved spring washer 146, radial ring 145, set screw 144, and dial 101 is shown. FIG. 18 also depicts the assembly of screws 153a and 153b, instrument attachment component guide 108 (with screw receiving holes 152a and 152b), cylindrical receiving stopper 151, and screw 133. FIG. 18 shows instrument attachment component 107 is attached to sliding carriage 149 through hole 150. FIGS. 10 and 18 show that as dial 101 is turned, intermediate components 145-148 (shown in FIG. 18) cause carriage component 149 to glide along elongated channel 103 (along the z-axis), together with instrument attachment component 107. It follows that any instrument attached to instrument attachment component 107 would also travel along the z-axis when the position of instrument attachment component 107 is adjusted by rotating dial 101.

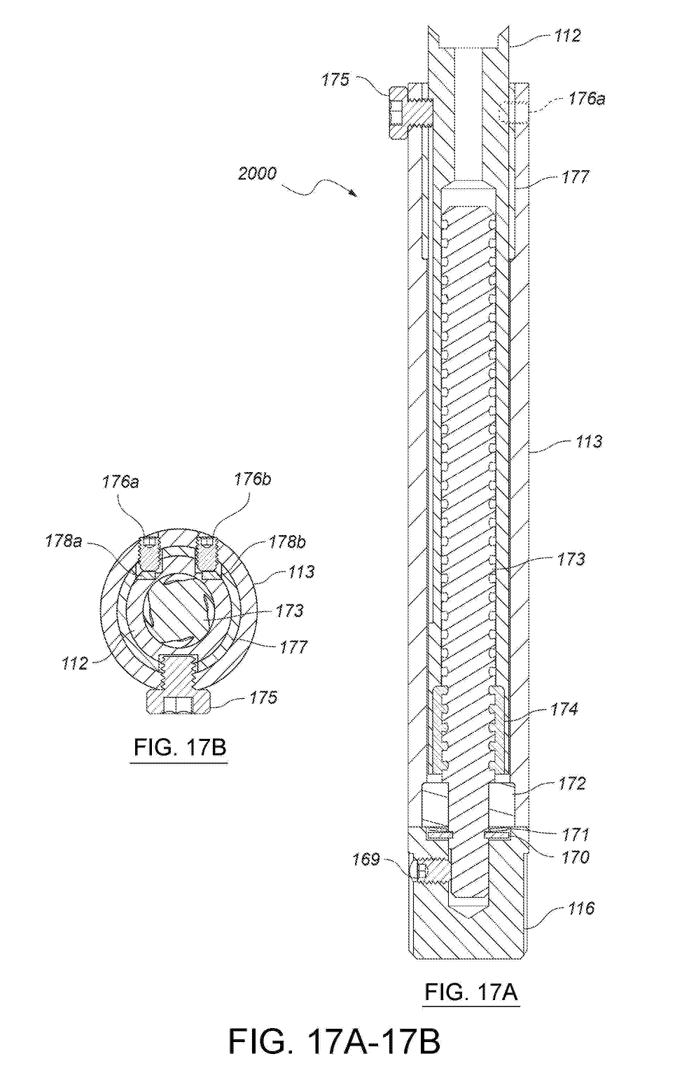

FIG. 3 shows an exploded view of stereotactic apparatus 100, in which the attachment of guiding arm 1000 to positioning arm 2000 is shown to be accomplished by securing screw 133 of guiding arm 1000 to receiving socket 134 of positioning arm 2000. FIG. 3 also shows that positioning arm 2000 traverses a cylindrical opening through upper collar 115 of cross clamp 132. FIG. 15 shows a partially exploded view of positioning arm 2000, in which the assembly of collar 174, threaded shaft 173, bushing 172, curved spring washer 171, radial ring 170, set screw 169, and dial 116 is shown. FIG. 15 also shows outer nested component 113 and inner nested component 112 of positioning arm 2000. FIG. 16 shows the assembly of inner 112 and outer 113 nesting components of positioning arm 2000. Specifically, screw 175 and set screws 176a and 176b traverse outer nested component 113 and inner stabilizing collar 177. The set screws 176a and 176b then contact supporting elements 178a and 178b, respectively, which in turn rest on the flat portions of elongated L-shaped grooves 179a and 179b, respectively. This arrangement allows supporting elements 178a and 178b (and screw 175) to constrain motion of inner nesting component 112 of positioning arm 2000, and adds to the stability and control of its telescoping motion. Cross-sectional views of positioning arm 2000 are depicted in FIGS. 17A and B.

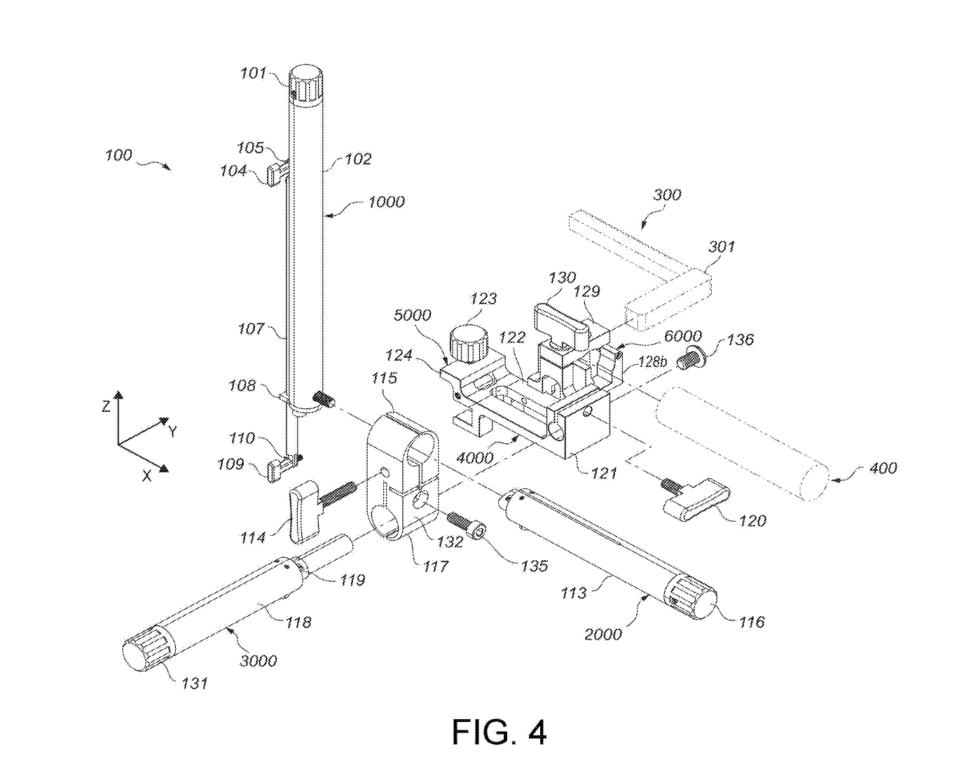

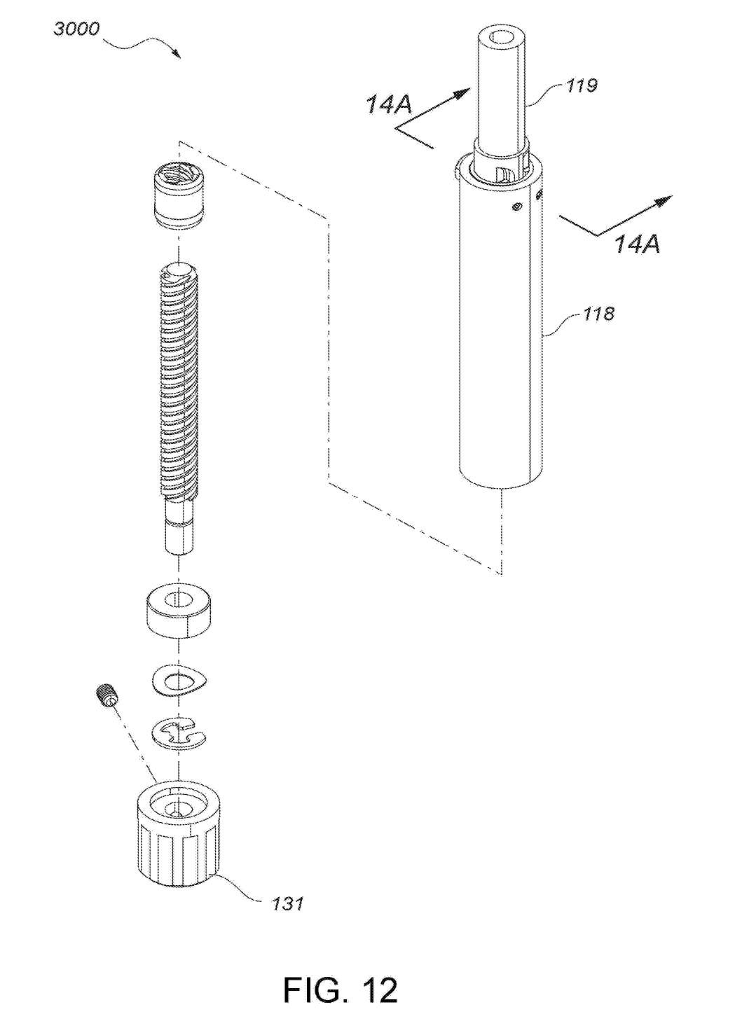

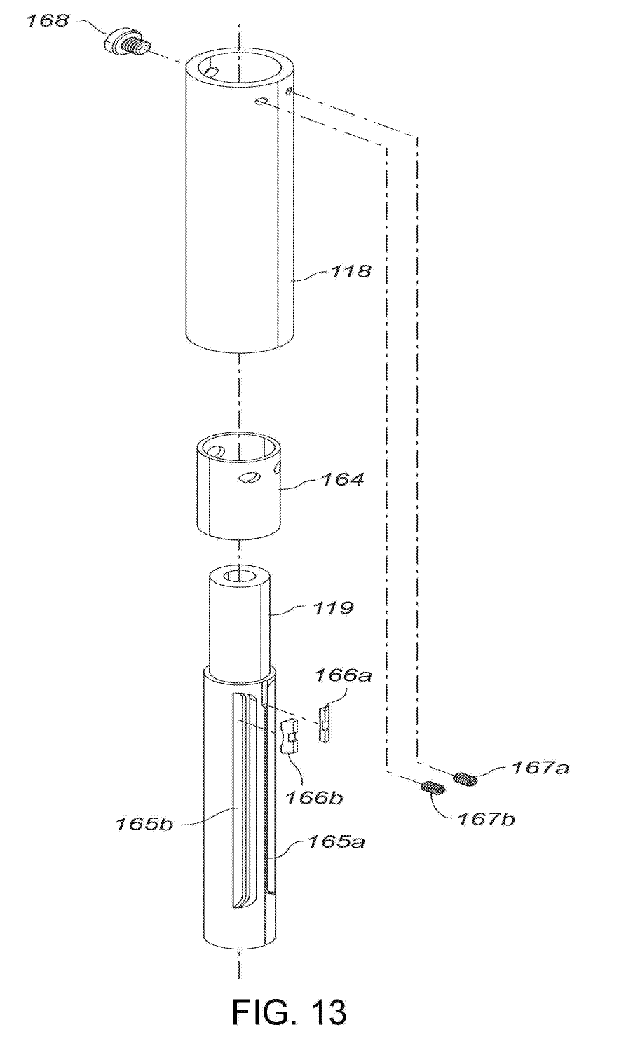

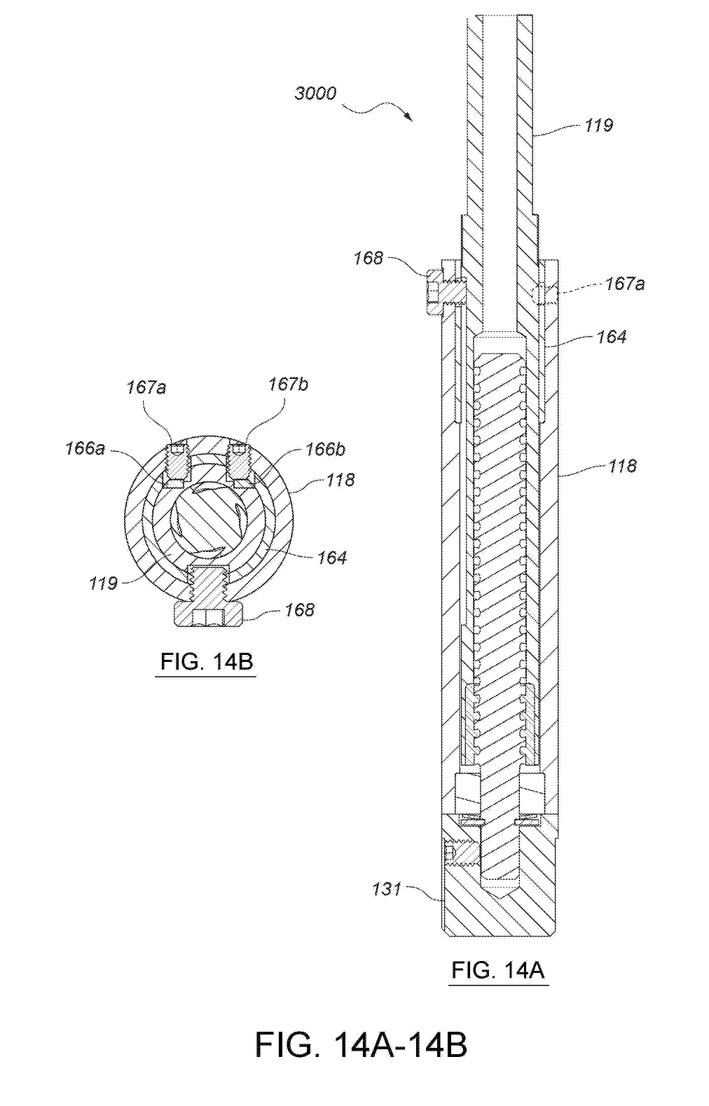

In addition to guiding arm 1000 and positioning arm 2000, FIG. 3 also shows connecting arm 3000 of stereotactic apparatus 100 with outer nested element 118 and inner nested element 119. FIG. 3 shows connecting arm 3000 traverses the cylindrical opening of lower collar 117 of cross clamp 132. FIG. 3 also shows that connecting arm 3000 traverses a cylindrical opening in clamp 121, and is fastened to end screw 136. An alternate view of these components is demonstrated in FIG. 4. FIG. 4 also depicts knob 120 and screw 135, which can each be tightened to secure connecting arm 3000 in clamp 121 and lower collar 117 (of cross clamp 132), respectively. FIG. 13 shows the assembly of inner 119 and outer 118 nesting components of connecting arm 3000. Screw 168 and set screws 167a and 167b traverse outer nested component 118 and inner stabilizing collar 164. Set screws 167a and 167b then contact supporting elements 166a and 166b, respectively, which in turn rest on the flat portion of elongated L-shaped grooves 165a and 165b, respectively. This arrangement allows supporting elements 166a and 166b (and screw 168) to constrain motion of inner nesting element 119, and adds to the stability and control of its telescoping motion. Cross-sectional views of attaching arm 3000 are depicted in FIGS. 14A and B.

FIG. 3 also shows a view of securing arm 4000, which includes clamp 121, body 122, and retractor attaching clamp 5000. Retractor attaching clamp 5000 is formed by knob 123, stabilizing screw 126 (which passes through upper lip 124 of clamp 5000), upper stabilizing tabs 125a and 125b, and lower stabilizing tabs 127a and 127b. An exploded view of securing arm 4000 is shown in FIG. 21. In this view, incorporation of set screw 162 and rod 161 in the context of the other components of the clamp can be seen.

FIG. 3 further shows side clamp 6000 of stereotactic apparatus 100. Side clamp 6000 includes tray arms 128a and 128b, and hinged top 129. Hinged top 129 includes an opening through which a portion of an object clamped by side clamp 6000 (such as elongated object 400 shown in FIG. 1) can be viewed.

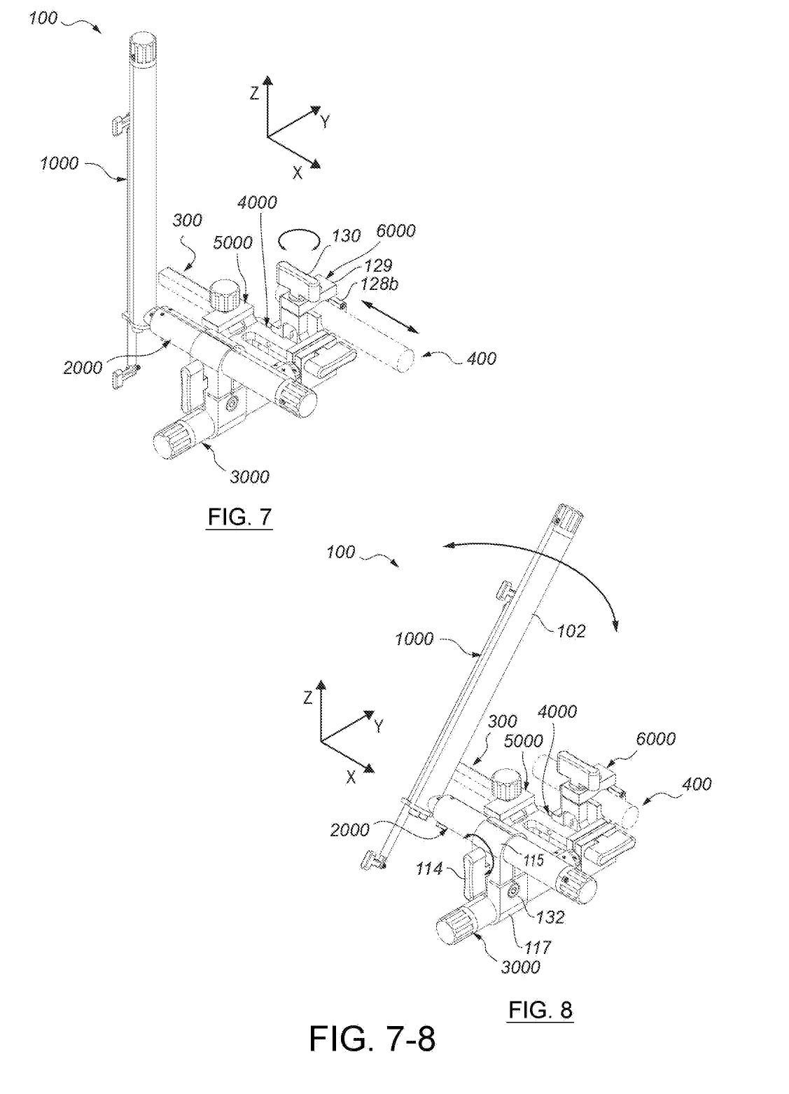

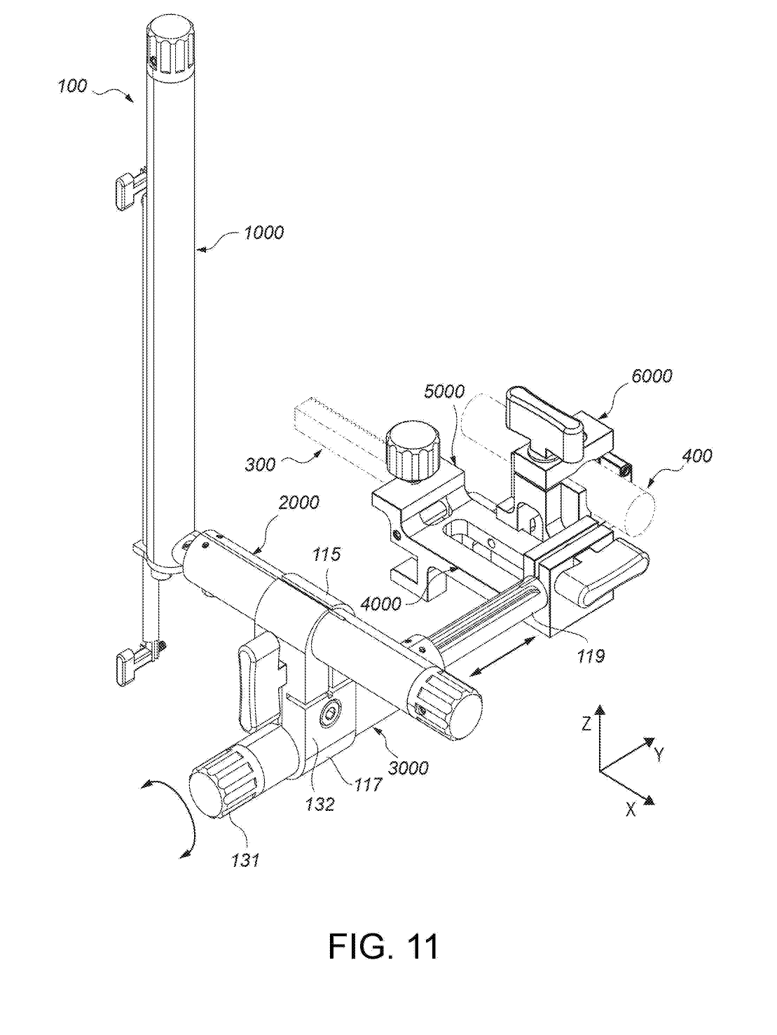

Turning now to the various possible adjustments and orientations of the arms (and components thereof) of stereotactic apparatus 100 shown in FIGS. 5-11. FIG. 5 shows rotation of knob 114 loosens upper collar 115 of cross clamp 132, thereby allowing adjustment of the position of positioning arm 2000 along the x-axis. FIG. 8 shows that rotation of knob 114 (and associated loosing of upper collar 115 of cross clamp 132) allows for rotation of positioning arm 2000 along the x-axis, which translates into motion of guiding arm 1000 along the y-z plane. FIG. 6 shows that rotation of screw 135 results in loosening lower collar 117 of cross clamp 132, which allows for adjustment of the position of positioning arm 2000 along the y-axis. FIG. 9 shows that rotation of screw 135 (and associated loosening of lower collar 117 of cross clamp 132) allows for rotation of cross clamp 132 along the y-axis, which translates into motion of guiding arm 1000 along the x-z plane. FIG. 7 demonstrates that rotation of knob 130 (and associated loosening of side clamp component 129) allows for adjustment of the position of cylindrical object 400 along the x-axis. FIG. 10 shows that rotation of dial 116 is associated with telescoping of positioning arm 2000 along the x-axis. FIG. 10 also shows that rotation of dial 101 is associated with motion of instrument attachment component 107 of guiding arm 1000 along the z-axis. FIG. 11 shows that rotation of dial 131 is associated with telescoping of connecting arm 3000 along the y-axis.

### Example 2

### Stereotactic Apparatus without Side Clamp

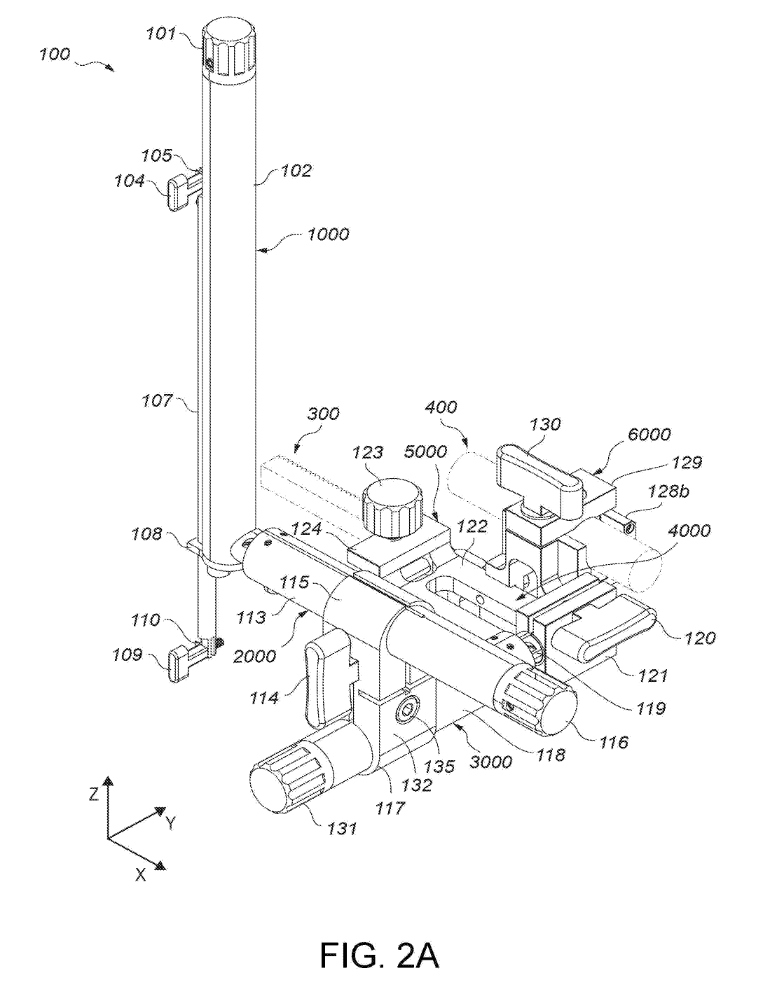

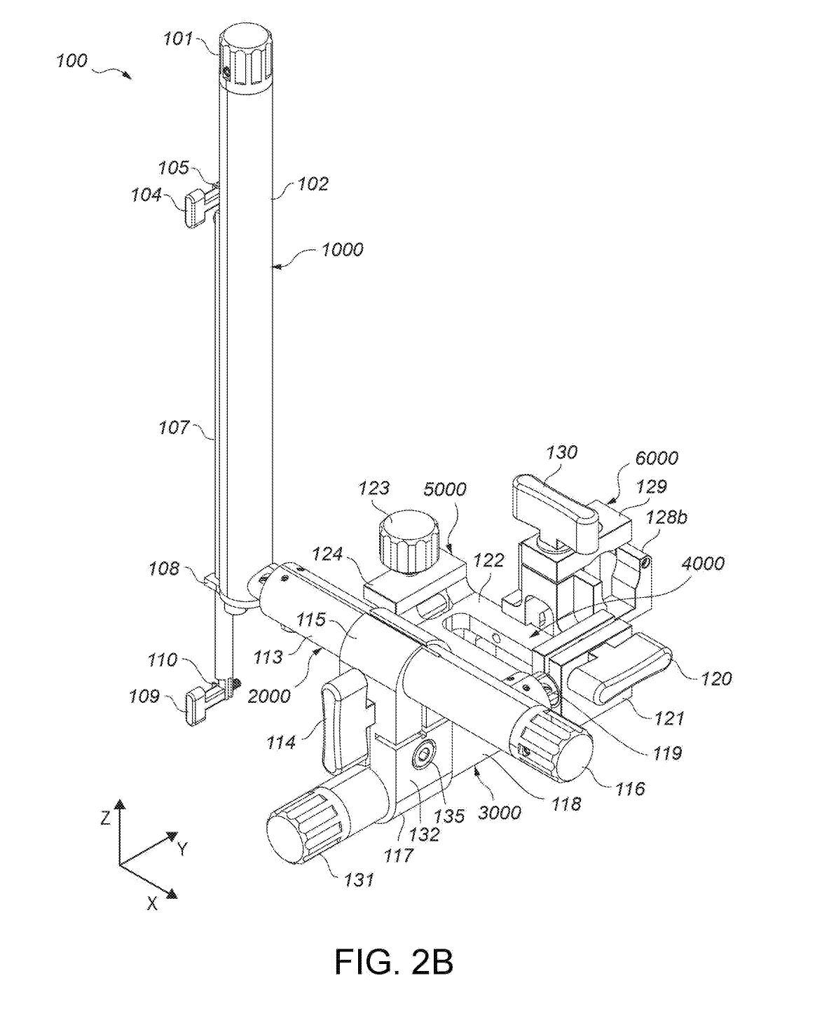

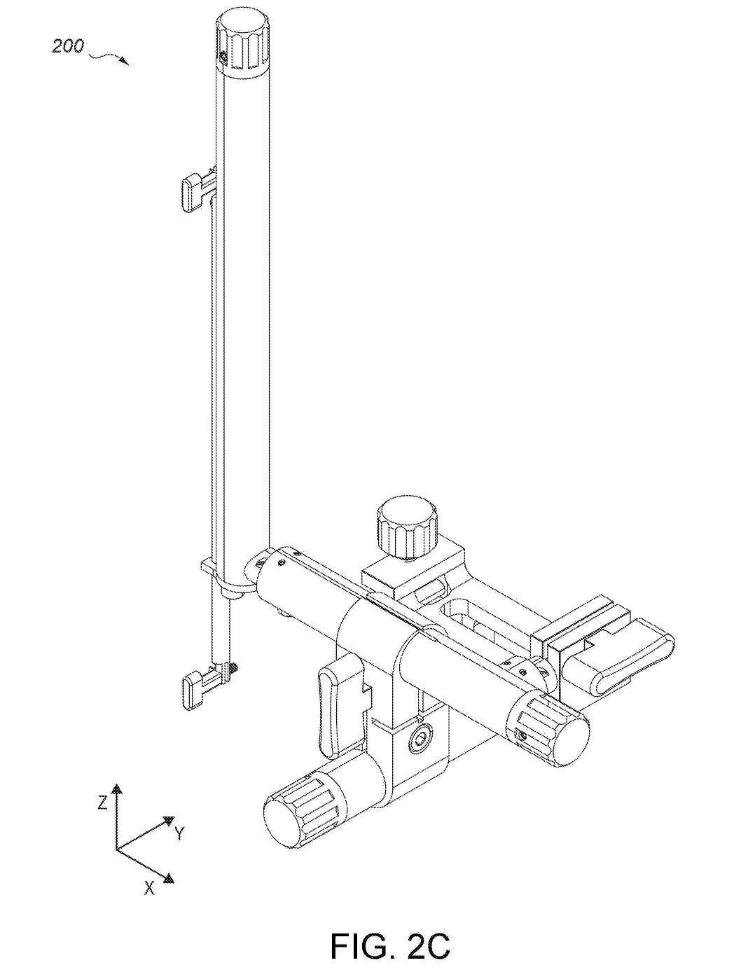

FIGS. 1C and 2C depict stereotactic apparatus 200, which includes the same components as stereotactic apparatus 100, with the exception of the side clamp 128 depicted in stereotactic apparatus 100. Stereotactic apparatus 200 also functions in the same way as stereotactic apparatus 100, with the exception of the functions that relate to side clamp 128.

### Example 3

### Surgical Procedure

A single level laminectomy can be performed on the L4 vertebral segment. Standard anesthetic/preoperatory techniques are used and the patient is positioned prone. A 4 cm incision is made at the midline above the L4 spinous process. Cutting electrocautery is used to cut the fascia and extend the incision to the spinous process, as well as achieving hemostasis of any small hemorrhages from the incision site. At this point a Weitlaner retractor can be used to keep the incision open. A bilateral sub-periosteal dissection is performed carefully by elevating the muscles and periosteum off of the lamina. Cutting electrocautery is used to facilitate the dissection. The spinous process is then removed using a Leksell rongeur. A high-speed drill is used to thin the lamina laterally. The lamina is then lifted and the ligamentous attachment is cut to release the lamina. Kerrison rongeurs are then be used to extend the laminectomy or clean up any left over bone fragments. In this case, the Medtronic Mast Quadrant retractor system is used. The Weitlaner retractor is removed, and the Mast Quadrant retractor blades are inserted into the incision and attached to the retractor system flex arms. The retractor is opened rostrocaudally to achieve maximum tissue spread. The mediolateral retractor is used in order to keep muscle out of the field. A ˜2.5 cm dura incision is made using an #11 blade and a dural guide to prevent spinal cord injury. Using 4-0 Neurolon the dura is then tacked at the four corners of the opening to be able to visualize the nerve roots and facilitate injections. At this point, inventive device 100 is attached to the Mast Quadrant using clamp 5000. Coronal and saggital angles can be adjusted on the device depending on the spinal cord target using the adjustment mechanisms described above. In this case, the ventral horn is targeted, so a 90-degree (orthogonal) angle of the surgical instrument (needle, cannula, etc) to the spinal cord is established. The surgical instrument (needle, cannula) can now be attached to the device. Using the dials of the device, rostrocaudal and mediolateral movement can be achieved to find accurate placement to the target. The surgical instrument is then positioned into the spinal cord using the ventral rostral movement provided by dial 101 to the appropriate depth. Imaging (CT, MRI, Ultrasound, and the like) can be used to help position the device in all planes (coronal and saggital angle, rostrocaudal, mediolateral and dorsoventral positioning). When the surgical instrument (needle) is in position, the therapeutic agent (neural progenitor cells) can be infused into the spinal cord target. The surgical instrument is then returned to the starting position and can then be repositioned for subsequent injections. Once all of the injections/infusions are completed, the surgical instrument can be removed, followed by the device. The dura tacks can then be cut and the retractor system removed. The incision can then be closed in four layers. The dura is closed with a running stitch using a 4-0 neurolon. Once it's closed, a valsalva maneuver can be performed to ensure it's watertight and there's no cerebrospinal fluid leakage. The deep muscle layer is closed with a 0 Vycril suture as well as the Muscle fascia. The dermal layer is closed using a 3-0 vycril and finally the skin is closed using a locked running stitch with 2-0 nylon.

### Example 4

### Stereotactic Device with Cannula

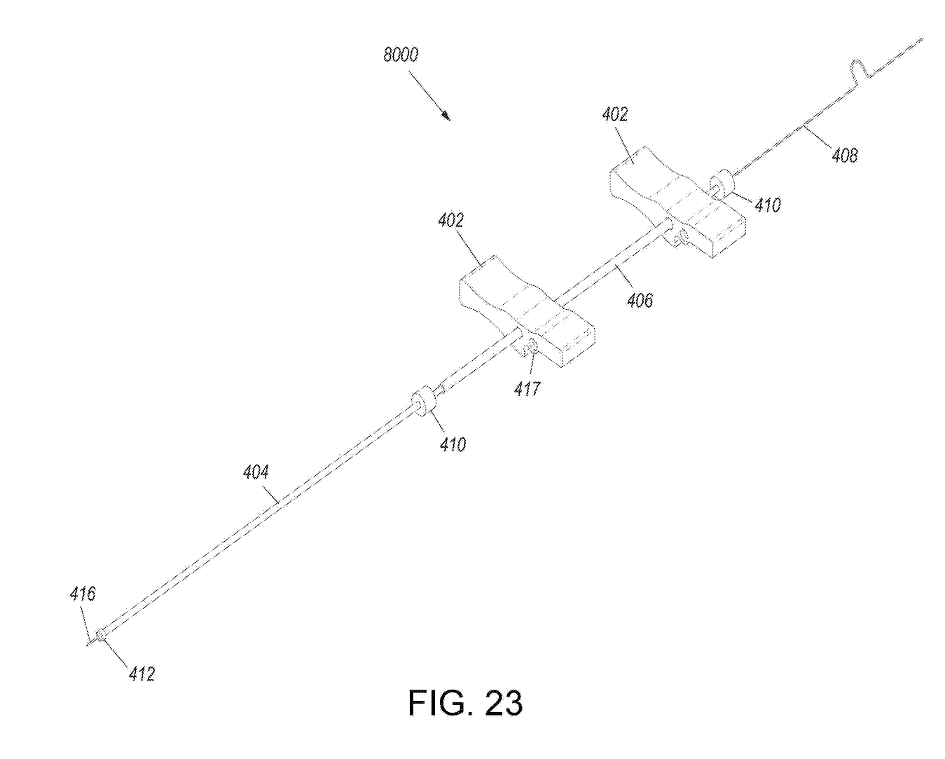

FIG. 23 depicts an example of a floating cannula system 8000 that may be attached to the guiding arm 1000 or other portion of a stereotactic apparatus 100 as disclosed herein. In some embodiments, the floating cannula system 8000 will include a base cannula 406 that has two support tabs 402 that are securely mounted to the base cannula 406. In some embodiments, the support tabs 402 may be utilized to connect the base cannula 406 to a stereotactic apparatus 100. In some embodiments, the support tabs 402 are spaced apart as shown in FIG. 23. In alternative embodiments, the support tabs may be closer together. In some embodiments, it may be advantageous to space the support tabs 402 so that they effectively stabilize the base cannula 406, in view of the length of the base cannula. In some embodiments, the base cannula 406 may only contain one or no support tabs 402 and instead may be connected directly to a guiding arm 1000 of a stereotactic apparatus 100. In some embodiments, the support tabs 402 may contain attachment sockets 417 (FIG. 23) that are configured to receive pins from a connector or guiding arm 1000 of the stereotactic device.

The base cannula 406, in some embodiments, may have a proximal end and a distal end, wherein the proximal end is closer to the top portion of the cannula system 8000. The base cannula 406 may contain a floating cannula 404 inside the lumen of base cannula 406. The floating cannula 404, in some embodiments, is restrained from movement by its engagement with base cannula 406 except that it may slide in both directions along the longitudinal axis of base cannula 406.

In order to limit the distance the floating cannula 404 may travel in both directions along the longitudinal axis of the base cannula 406, the floating cannula 404 may include stoppers 410. The stoppers 410 may be attached to the floating cannula 404 above and below the proximal and distal ends of the base cannula 406 respectively, when the floating cannula is engaged in the base cannula, as shown in FIG. 23. The proximal stopper 410 that is above the proximal end of the base cannula 406 will prevent the floating cannula 404 from falling out of the base cannula 406 (due to gravity) when positioned so that a portion of the floating cannula 404 extends beyond the distal end of the base cannula 406. The distal stopper 410 placed on the distal end of the floating cannula 404 restricts the floating cannula 404 from being pushed too far upward with respect to the base cannula 406, and may provide resistance for allowing a needle 416 to puncture a patient's tissue, once the distal stopper 410 contacts the distal end of the base cannula 406, as the base cannula is lowered toward an injection site on the patient.

In order to puncture tissue and deliver a substance to a patient, the floating cannula 404 may include a hollow needle 416 and a tissue stopper 412. The floating cannula system 8000 may be lowered down by the guiding arm 1000 of stereotactic device 100, until the needle 416 contacts the tissue of a patient. Then, once the needle contacts the patient's tissue, the floating cannula 404 will be pushed upwards with respect to the base cannula 406. As indicated above, the floating cannula 404 may include a distal stopper 410 that eventually contacts the distal edge/end of the base cannula 406 as the base cannula 406 is lowered towards the patient by the guiding arm 1000. Once the distal stopper 410 contacts the distal edge/end of the base cannula 406, the stopper will provide resistance and the floating cannula 404 will no longer move up with respect to the base cannula 406. Stopper 410 may be any piece of material attached to the cannula 404 that prevents the base cannula 406 from sliding over or past the stopper 410 (FIG. 23). Stopper 410 thus could be configured as a bump, donut, cylinder, tab, square, wedge, or otherwise shaped obstruction large enough to prevent the floating cannula 404 from moving beyond a certain limit with respect to the base cannula 406. The stoppers 410 may be made of any suitable material, including plastics, rubbers, thermoplastics, glass, metal, wood or any others. In some embodiments, a rubber stopper 410 may be utilized to prevent damaging the base cannula when it come into contact with the stopper 410.

Then, proceeding with the process of injection, if the guiding arm 1000 moves the base cannula 406 farther down towards the targeted tissue site, the needle 416 will puncture the targeted tissue site. The needle 416 will penetrate the tissue until the tissue stopper 412 contacts the tissue site. The tissue stopper 412 may be any suitable shape or size to prevent the needle 416 from entering further into the tissue. The tissue stopper 412 may be wedge shaped, disc shaped, or any other suitable shape. The tissue stopper 412 may be included on only part of the circumference of the needle 416 and other suitable arrangements. The tissue stopper 412 may be appropriately spaced/positioned with respect to the tip of the needle 416 to allow for the correct injection depth based on the particular procedure. In some embodiments, the tissue stopper may be movable with respect to the needle, in order to allow for different injection depths required for different procedures.

Once the needle 416 enters the body of the patient, and the tissue stopper 412 contacts the patient's tissue, the base cannula 406 may be pulled upwards. This may be accomplished by moving the guiding arm 1000 upwards, which would in turn move the attached base cannula 406 upwards. This would move the distal edge/end of the base cannula 406 away from and upwards with respect to the distal stopper 410 and provide a space or distance between the distal edge of the base cannula 406 and the distal stopper 410. This will allow the floating cannula 404 a limited range of movement along the longitudinal axis of the cannulas. Accordingly, if the patient moves in a direction along that axis, the floating cannula 404 will move with respect to the base cannula 406, without causing damage to the patient. The travel of the floating cannula 404 along the longitudinal axis will be limited by the spacing of the distal and proximal stoppers 410 relative to the length of the base cannula 406. This system 8000 will advantageously allow the needle 416 to be precision injected into the tissue site, and then allow some freedom of movement along the longitudinal axis, once the base cannula 406 is pulled back (further away from the tissue site).

In some embodiments, the substance to be injected into the patient will be delivered by a delivery tube 408 that may be connected to an external reservoir and pump. The reservoir will be connected to the delivery tube 408 which may then run along the length of the entire system 8000, within the lumens of the base cannula 406 and floating cannula 404, and connect to the needle 416 (or include a penetrating tip that serves as the needle 416). In some embodiments, the delivery tube 408 may only connect to the floating cannula 406 and deliver the substance to inside the lumen of the cannula 406.

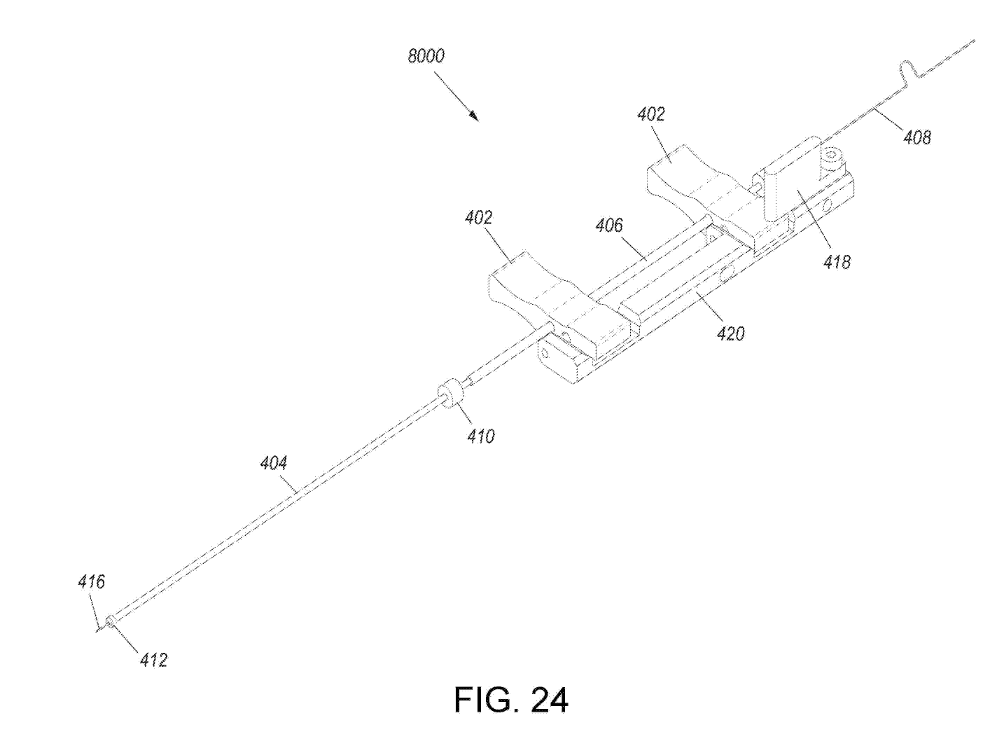

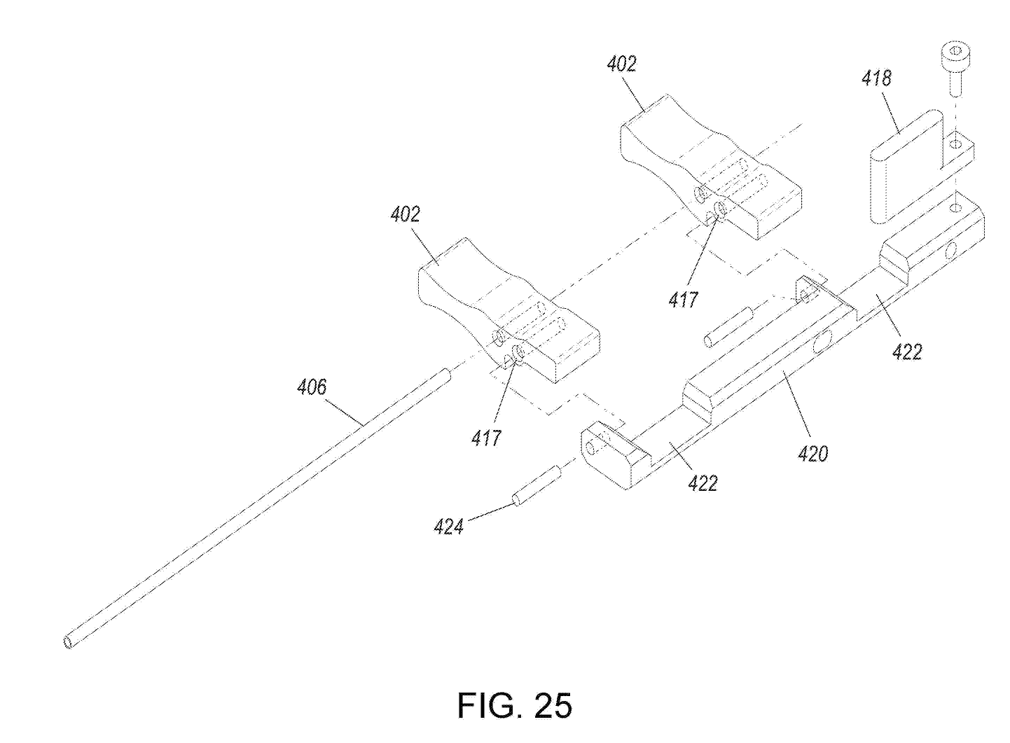

FIG. 24 illustrates the floating cannula system 8000 connected to a connector 420 configured to connect the system 8000 to a stereotactic device 100. The connector 420, in some embodiments, includes a tab lock 418 that mounts the support tabs 402 to the connector 420. In some embodiments, the tab lock 418 creates an interference fit. In the illustrated embodiments, the sockets 417 of the support tabs 402 are inserted onto pins 424 that are included in the connector 420 (FIG. 25). The pins 424 then provide translational restraint of the cannula system 8000 in a plane perpendicular to the longitudinal axis of the cannulas. Then, the support tabs 402 may be rotated into place inside a space or indentation 422 in the connector 420 by rotation around the pins 424 that are attached to the connector 420. Once the support tabs 402 have been rotated into place, a tab lock 418 may be rotated into place, (based on a rotation or sliding action or other suitable mechanical means) to block the tabs 402 from rotating back out of the spaces 422 in the connector 420 (FIGS. 24 and 25). In other embodiments, the tabs 402 may be attached to connector 420 through other suitable mechanical devices, including buckles or other mechanical connections.

FIG. 25 depicts an exploded view of the connector 420, along with the base cannula 406 and tabs 402. As depicted, the tabs 402 include sockets 417, in which pins 424 may fit. The pins 424 may be attached to the connector 420 and positioned so that when the tab sockets 417 are positioned onto the pins 424 the tabs 402 may be rotated into the spaces or indentations 422 in the connector 420. The spaces or indentations 422 in the connector may be configured to accommodate the support tab ends, so that the support tabs will be restricted in the direction parallel to the longitudinal axis of the cannulas. In this embodiment, the spaces or indentations 422 are illustrated to include a square shape, so that they may accommodate a square end of the tabs 402 that may be rotated into place about pins 424 and locked there with the tab lock 418.

FIG. 26 illustrates an exploded view of the system 8000 with the base cannula 406 connected to the connector 420.

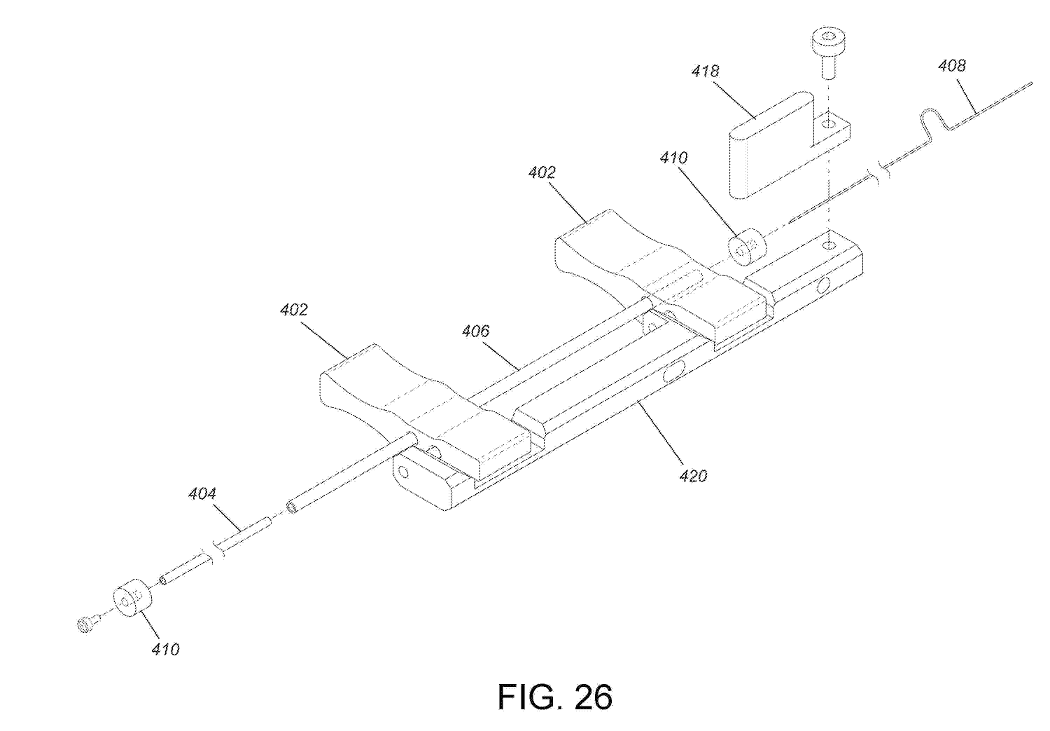

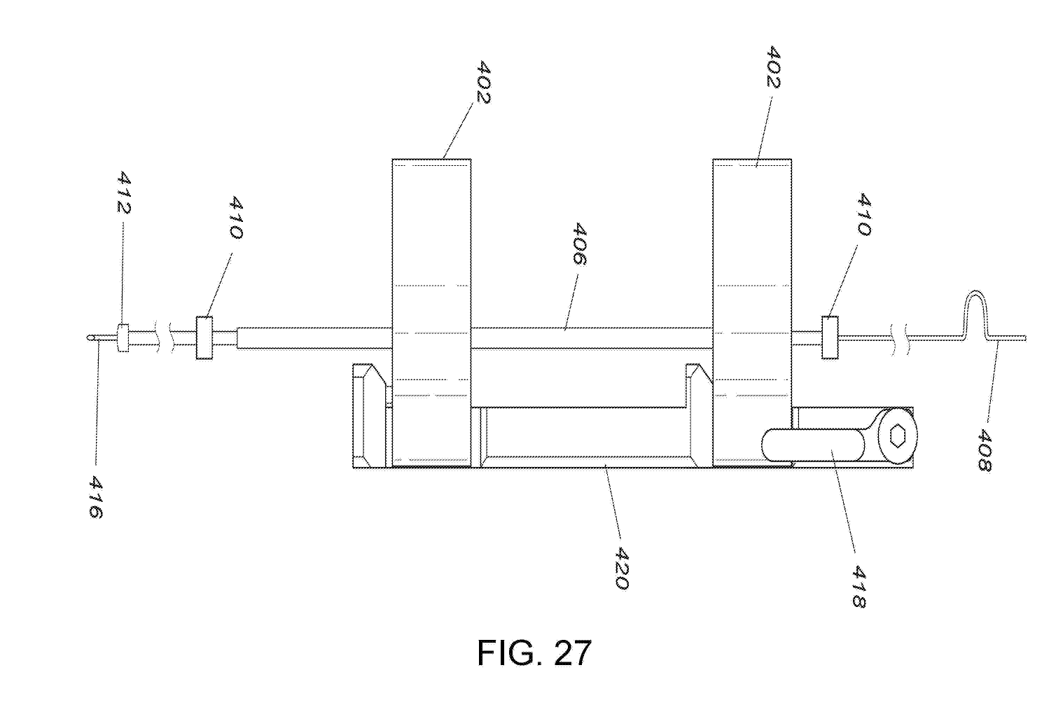

FIG. 27 illustrates a side view of the system 8000 with the base cannula 406 attached to the connector 420.

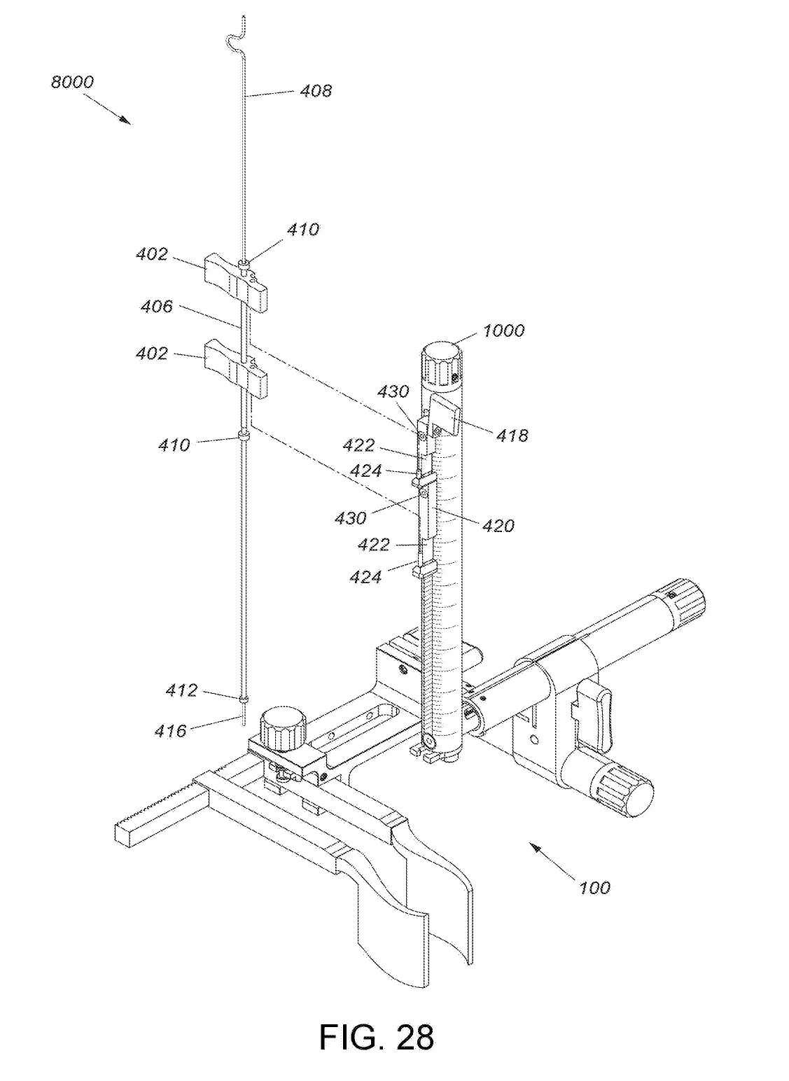

FIG. 28 illustrates an embodiment in which the floating cannula system 8000 and connector 420 are connected to the guiding arm 1000 of the stereotactic device 100. The connector 420 may be attached to the guiding arm 1000 with screw 430 (alternative means of attachment, as described herein, may be separately or additionally used). FIG. 28 illustrates the connector 420 with the pins 424 inserted and the floating cannula system being moved towards the pins, so that the sockets 417 of the tabs 402 may be mounted on the pins 424. However, in FIG. 28, the support tabs 402 have not yet been rotated inside spaces or indentations 422 of the connector 420. This allows the support tabs 402 to slide onto pins 424 when first placed on the pins 424 in an orientation that is rotated approximately 90 degrees from the orientation they assume once secured.

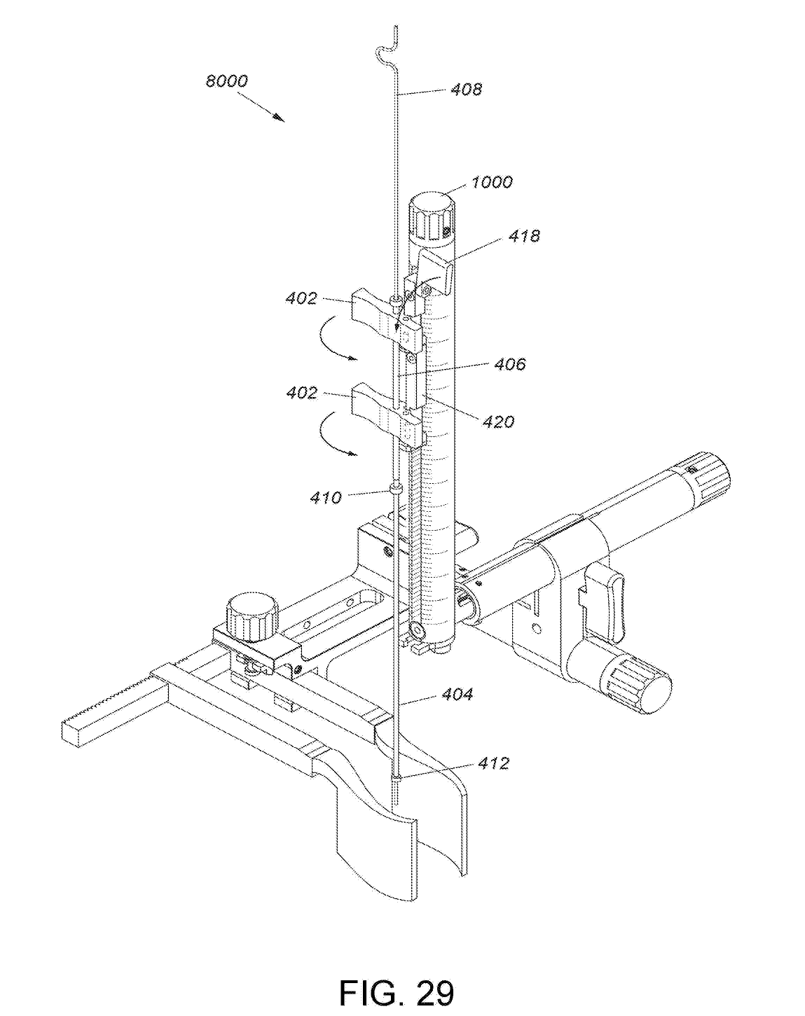

FIG. 29 illustrates the tabs 402 mounted onto the pins 424 (shown in FIG. 28). In some embodiments, the user may grip the support tabs 402 and then move the bottom opening of each of the sockets 417 above the pins 424, followed by sliding the support tabs 402 down the pins 424. Accordingly, the pins 424 will hold the support tabs 402 in place and only allow them to slide up and down along pins 424, or rotate about pins 424.

Once the tabs 402 have been placed on the pins 424 in the orientation shown, the tabs may be rotated 90 degrees as shown in FIG. 29, so that the edges of the tabs reside in the spaces or indentations 422 (shown in FIG. 25). Once the support tabs 402 have been rotated into place, the top and bottom of the spaces or indentations 422 will restrain the tabs 402 and therefore floating cannula system 8000 from moving up or down or in a direction along the longitudinal axis of the cannulas. FIG. 29 illustrates with an arrow the direction that the support tabs 402 have been rotated.

Once the support tabs 402 are rotated into spaces or indentations 422, the tab lock 418 may be rotated down to create an interference fit, which prevents the support tabs 402 from rotating back out. In this embodiment, because support tabs 402 are securely attached to the base cannula 406, only one tab lock 418 may be required to block rotation of one of the support tabs 402. In other embodiments, both support tabs 402 may have tab locks 418 that block their rotation out of spaces or indentations 422.

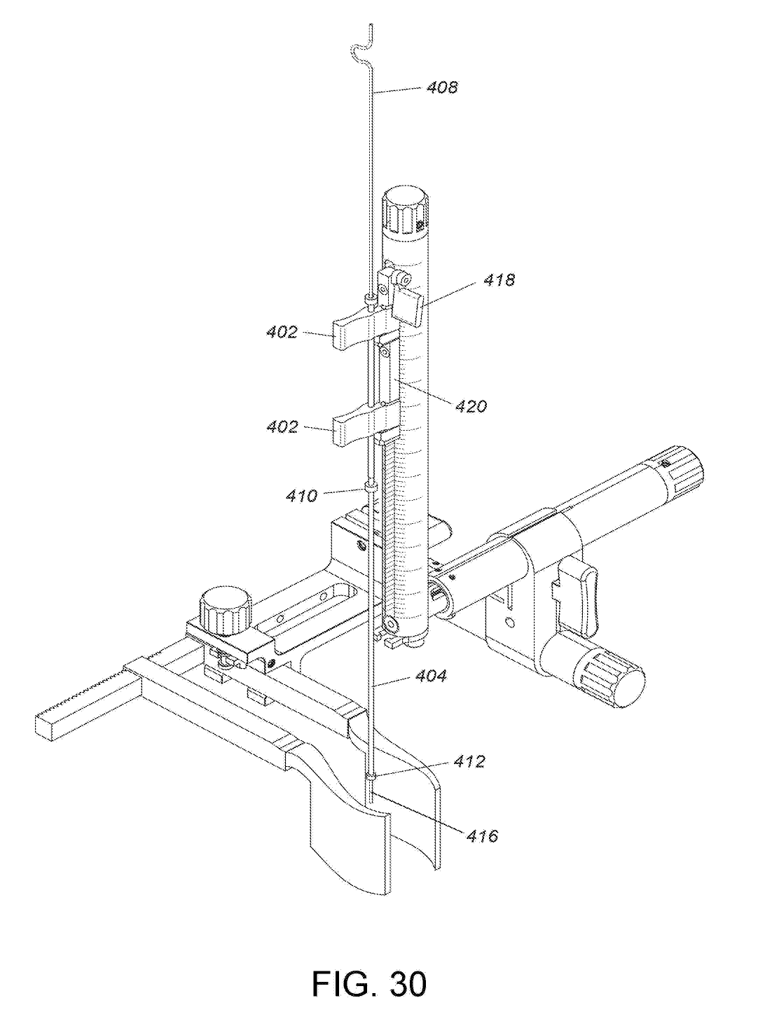

FIG. 30 illustrates the support tabs 402 and floating cannula system 8000 rotated into place in the spaces or indentations 422, and the tab lock 418 secured into place. In this configuration, the floating cannula system 8000 is securely attached to the connector 420 and guiding arm 1000 of the stereotactic device 100. As indicated above, the support tabs 402 are securely held to the connector 420 by the pins 424, spaces 422 and the tab lock 418. As described herein, other methods of attaching the floating cannula system 8000 to the guiding arm 1000 may be utilized. As described herein, the floating cannula system 8000 may be advanced towards a tissue site to bring the needle 416 in closer proximity to the site by lowering the guiding arm 1000.

The floating cannula system described above may be utilized for a variety of procedures that require a precision injection. Merely by way of non-limiting examples, precision injections may be performed on a patient to introduce sustained release peptides, cells (including stem cells), vectors for gene therapy, or any other medically relevant substance described herein. The injections may be made to the spinal cord parenchyma, other neurological structures, and other parts of the body, as described herein. In some embodiments, the floating cannula system is used to inject neural progenitor cells into the spinal cord of a subject. In some embodiments, the neural progenitor cells express glial cell line derived neurotrophic factor. In some embodiments, the subject is a human who has been diagnosed with amyotrophic lateral sclerosis (ALS).

### Example 5

### Syringe Pump

FIG. 31 depicts a partially exploded view of a syringe pump system 9000, in which a carpule assembly 501, a drive shaft 502, a coupling collar 503 and a motor assembly housing 504 can be seen. The motor 513 of syringe pump system 9000 is configured to cause rotatable drive shaft 502 to rotate. As shown in FIG. 32, the carpule assembly includes an elongated inlet port 508, an elongated outlet port 511, and a chamber 510 disposed between and in fluid communication with elongated inlet port 508 and elongated outlet port 511. FIG. 32 also shows an elongated plunger 509, which is configured to nest within elongated inlet port 508. As shown in FIG. 32, the pushing end of elongated plunger 509 is configured to form a substantially fluid-tight seal with chamber 510, and rotatable drive shaft 502 is configured to apply a drive force to the receiving end of plunger 509. With this configuration, plunger 509 can be pushed in the direction of outlet port 511 (FIG. 33), thereby expelling any liquid in chamber 510 through outlet port opening 512.

As shown in FIG. 31, coupling collar 503 is configured to connect on one end to motor housing assembly 504, and on the other end to carpule assembly 501.

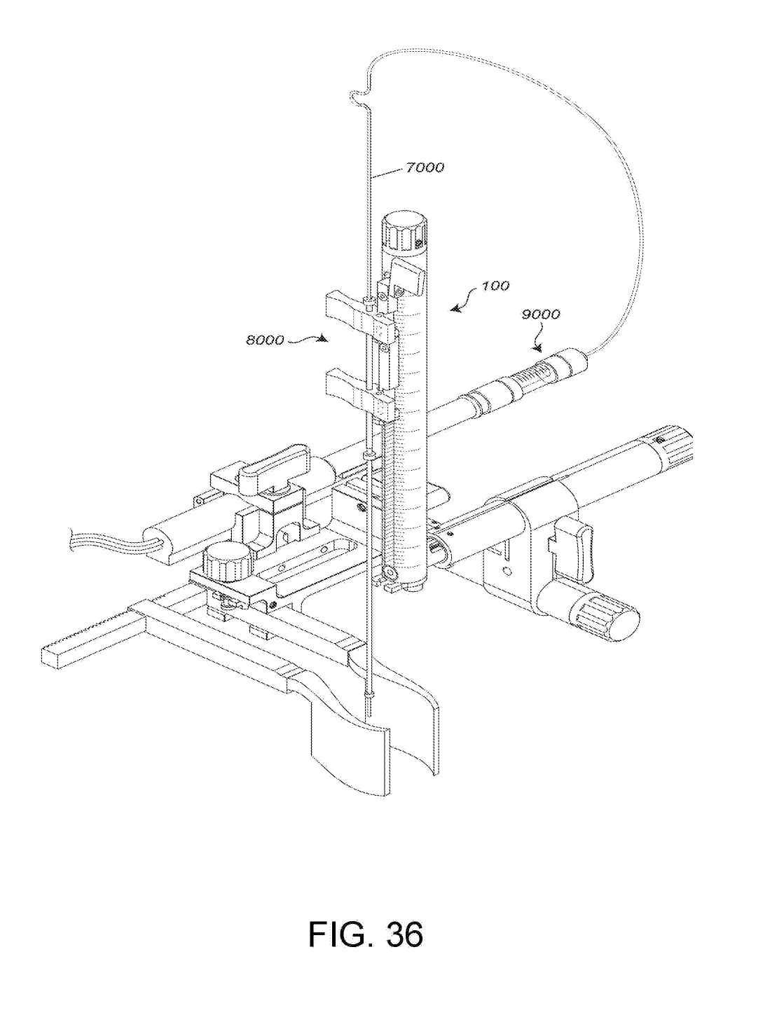

FIG. 36 depicts cannula delivery tube 7000 connected to syringe pump system 9000 and floating cannula system 8000.

As shown in FIG. 37 cannula delivery tube 7000 can be connected to carpule delivery tube 10000 through Leur lock fittings 10003 and 10002. FIG. 37 also shows carpule delivery tube 10000 can be connected to syringe pump system 9000 through coupling collar 10001. As described herein, cannula delivery tube 7000 may be directly connected to a hollow needle on the tip of the floating cannula, by running through the lumens of the base and floating cannulas of the cannula system.

An inventive syringe pump system described herein can be used in conjunction with a floating cannula system described herein and a stereotactic device described herein, in order to deliver neural progenitor cells expressing glial cell line derived neurotrophic factor into a patient's spinal cord. For example, using the configuration shown in FIG. 37, once a laminectomy is performed and a section of the spinal cord is accessible (by performing the surgical method described above), the guiding arm of the stereotactic device can be used to advance the hollow needle of the floating cannula into the patient's spinal cord. Once the hollow needle is inserted into the patient's spinal cord, the base cannula can be retracted by retracting the guiding arm upward from the injection site, thereby allowing for travel of the floating cannula within the base cannula, along the longitudinal axis of the base cannula. Next, the syringe pump can be used to pump saline, which was preloaded in the carpule, through carpule delivery tube 10000, which was preloaded with neural progenitor cells expressing glial cell line derived neurotrophic factor, thereby advancing the cells and saline through cannula delivery tube 7000, and ultimately through the hollow needle of the floating cannula and into the patient's spinal cord. If necessary, this procedure can be repeated at the same injection site, or at a different injection site, by replacing the used carpule and carpule delivery tube with a new carpule and carpule delivery tube that have been preloaded with saline and cells, respectively, as described above. After one or multiple injections are performed, the cannula can be retracted by completely retracting the guiding arm of the stereotactic device from the surgical site, and the incision in the patient can be closed according to the surgical procedure described above.

Although the delivery of therapeutic cells to the spinal cord is specifically described in the example above, any liquid therapeutic substance (or imaging substance) could be delivered into the spinal cord, or other anatomical targets, using the cannulas, stereotactic devices, and syringe pump systems described herein.

### Example 6

### Imaging System

FIG. 38 depicts an exemplary embodiment of the inventive imaging system. As shown, the imaging system 12000 includes an ionizing radiation source 12004 and a digital radiation detector 12003, each coupled to opposing blades 12002 and 120001 of a tissue retractor. The radiation from the radiation source 12004 can be collimated to “focus” the radiation towards the radiation detector 12003 along the path of the anatomical target of interest, as shown. FIG. 38 demonstrates how the radiation source and detector can be positioned on either side of a subject's spinal cord. FIG. 39 shows a configuration of an ionizing radiation source and detector each coupled to a retractor blade of a retractor device.

In practice, when the retractor blades are engaged in an incision in a subject, the source and detector can be utilized to image almost any anatomical structure situated between them. The imaging information obtained by the detector can be transmitted (through wires or wirelessly) to a computing workstation capable of processing the raw data gathered by the detector and constructing an image for viewing on a video monitor of a computer or other device.

In one example, when injections are performed on a subject's spinal cord by utilizing a floating cannula system described herein and a syringe pump system described herein, each coupled to a stereotactic device described herein, the inventive imaging system can be used to image the spinal cord in the manner shown in FIG. 38 By imaging the spinal cord before and/or during the procedure, the needle of the cannula can be guided to the intended injection site (gray matter), based upon the landmarks described herein above, and can be inserted to the desired depth within the spinal cord (with or without the use of a tissue stopper on the cannula needle).

While examples of imaging the spinal cord are specifically disclosed herein, other anatomical sites of interest could also be imaged with similar results. Furthermore, the imaging device could be used in conjunction with any suitable cannula and/or syringe pump and/or stereotactic system, whether described herein or otherwise.

### Example 7

### Tissue Retractor Systems

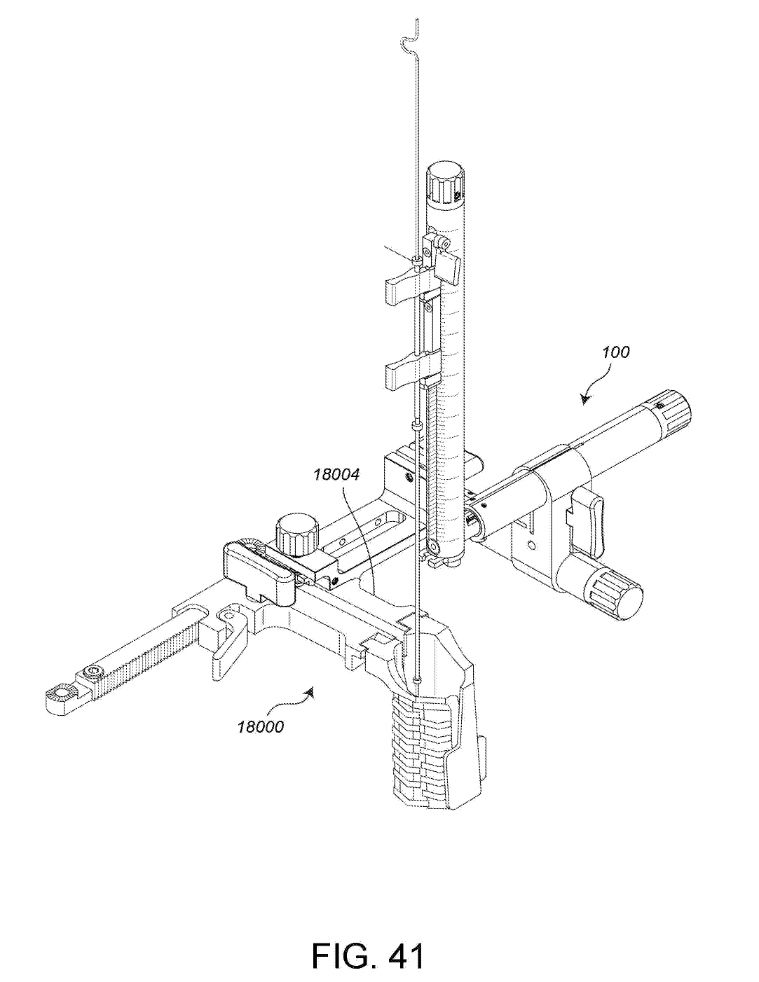

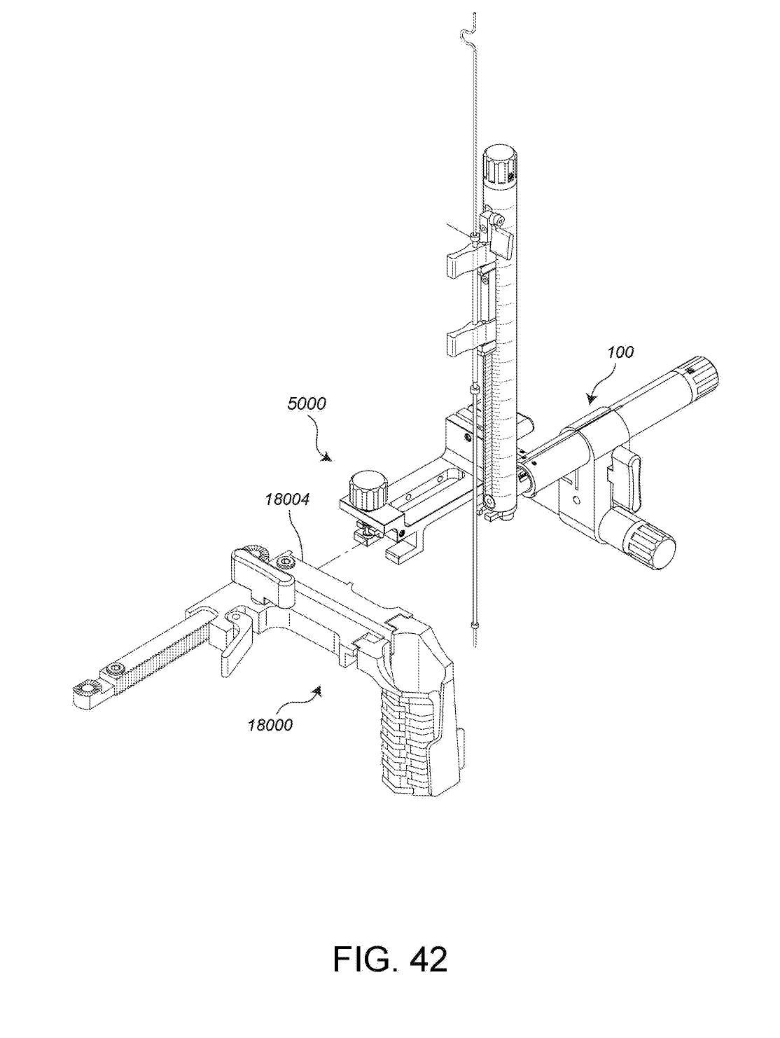

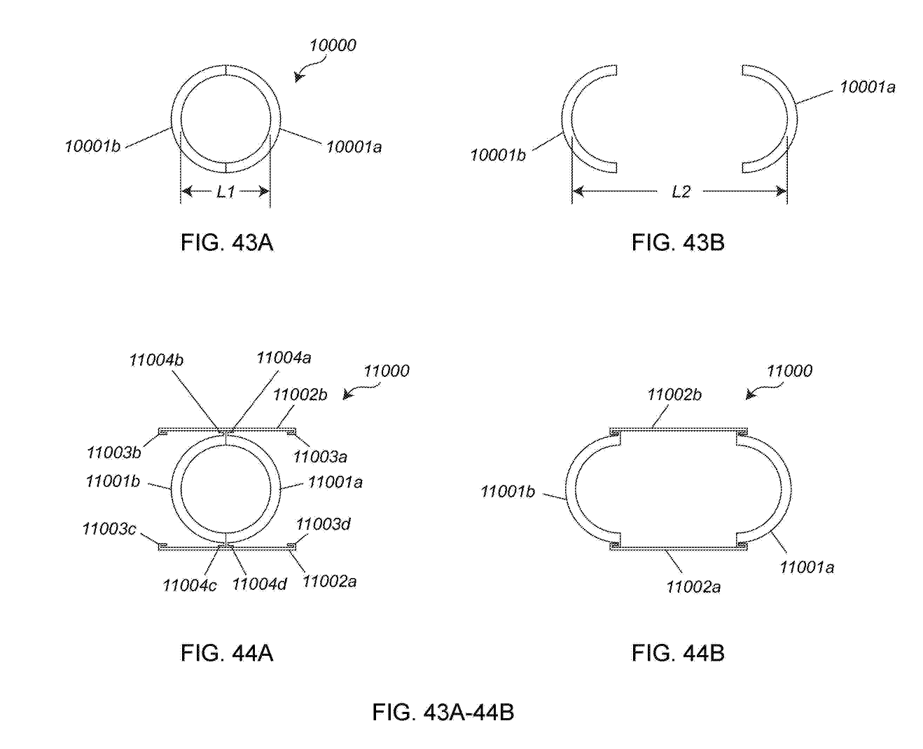

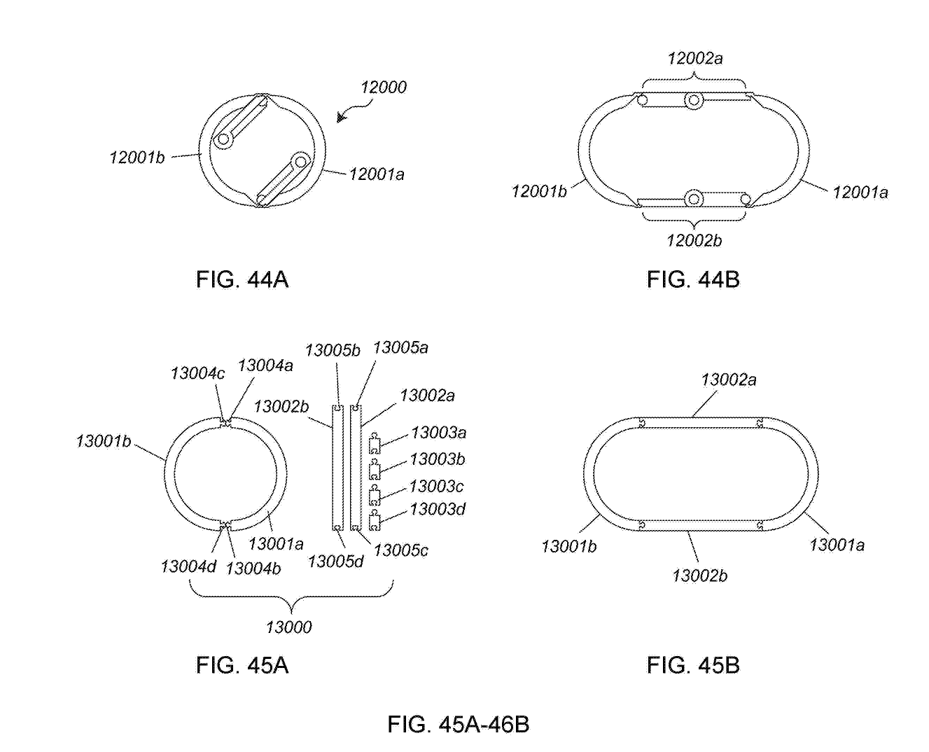

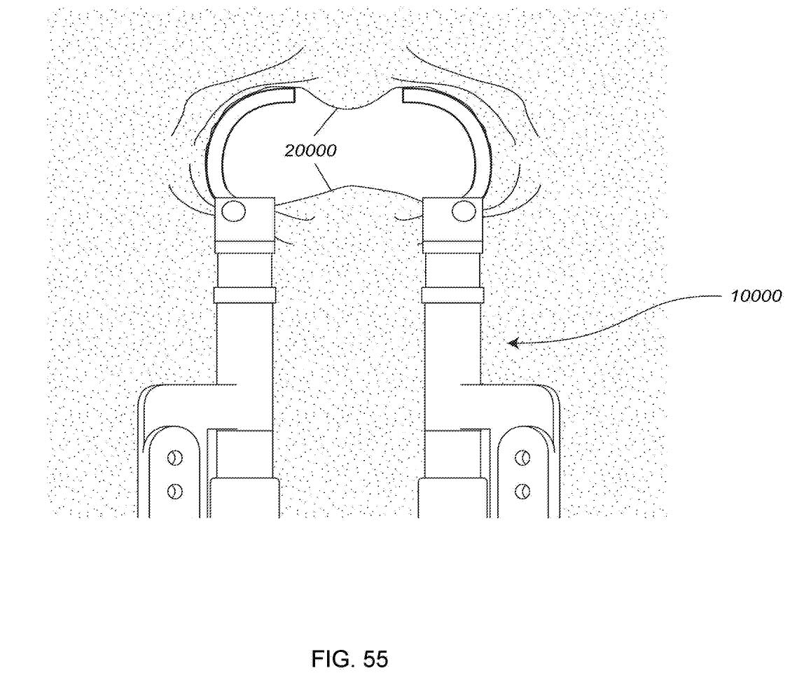

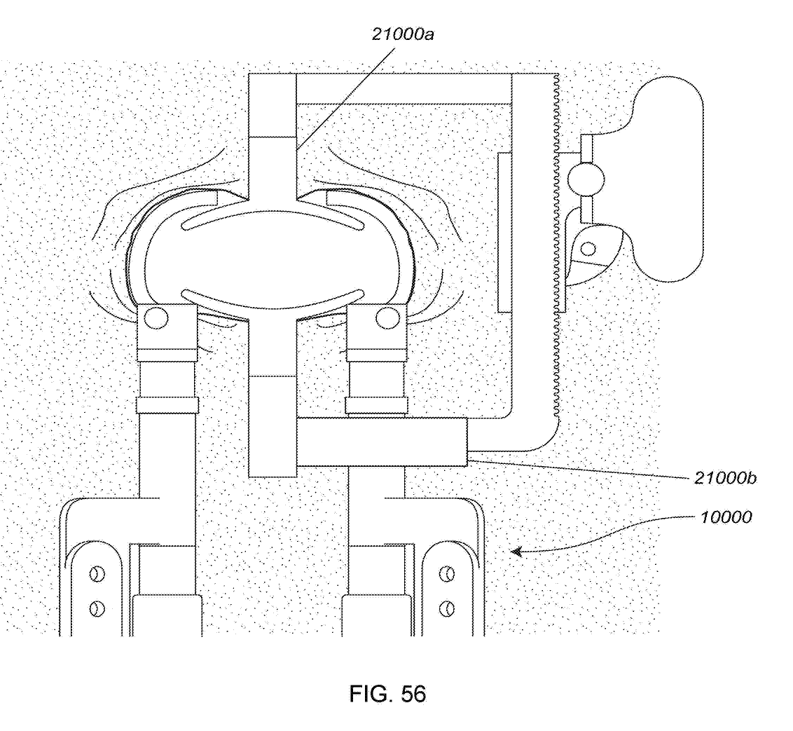

Tissue retractors consist of two opposing “blades” which are designed to be attached to arms of a retractor device. An example of a tissue retractor is shown in FIGS. 41 and 42, and a top-down view of rounded blades of a typical tissue retractor is shown in FIGS. 43A-44B. Although tissue retractor blades are usually effective at displacing and separating tissue along the axis on which they are separated from one another, tissue sometimes encroaches into the space provided by the blades from a perpendicular medial-lateral axis. An example of this type of tissue encroachment is depicted in FIG. 55, in which tissue retractor blades 10001a and 10001b of tissue retractor 10000 are separated, and tissue 20000 is encroaching between the blades. In order to avoid this type of tissue encroachment, additional retractor blades are often used. For example, FIG. 56 shows medial-lateral tissue retractor blades 20001a and 20001b utilized in conjunction with standard tissue retractor 10000. The medial-lateral tissue retractor is effectively preventing encroachment of tissue 20000. Unfortunately, using multiple sets of tissue retractor blades is cumbersome and relatively time consuming. Additionally, as indicated above, many tissue retractors are relatively unstable when deployed in certain regions of a subject's body, and are thus prone to slipping.

With the foregoing background in mind, various embodiments of tissue retractor systems of the present invention are designed to avoid tissue encroachment, while still allowing for efficient deployment, separation, and removal of the tissue retractor blades. In various embodiments, the tissue retractor systems of the present invention also allow for greater stability after deployment, compared to typical tissue retractors.

In various embodiments, the present application discloses novel tissue retractor apparatuses, systems and methods of use thereof. In some embodiments, the novel tissue retractor apparatuses are configured to work with stabilizing apparatuses, cannula systems and apparatuses, and syringe pump systems and apparatuses, such as those described in PCT/US15/58134, which is hereby incorporated herein by reference in its entirety as though fully set forth. While one of skill in the art would readily appreciate that there are many possible applications of the systems and apparatuses described herein, certain embodiments are especially useful for procedures performed on or around the spinal cord, including delivery of cutting edge cellular and molecular therapies thereto.

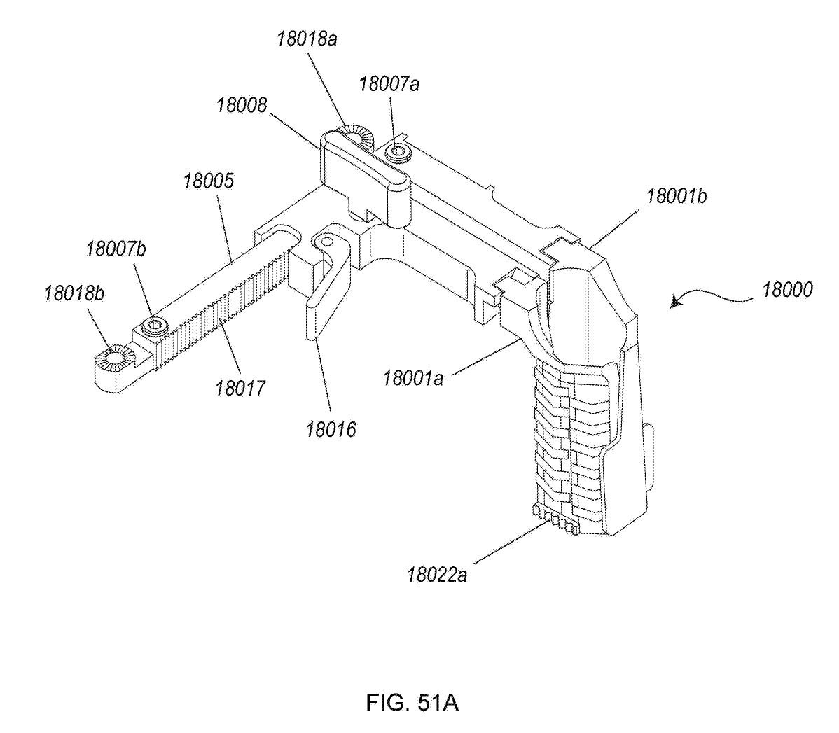

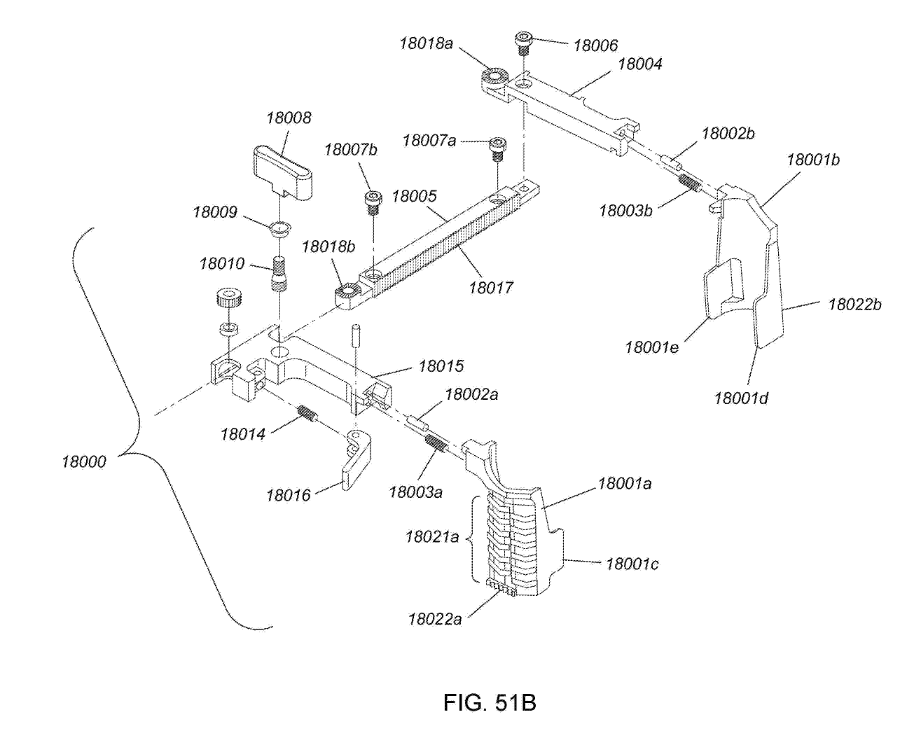

Although numerous embodiments of tissue retractors are described herein, there are certain common features. First, each tissue retractor system includes at least two opposing tissue retractor blades designed to separate tissue. A “blade” as used in the context of the tissue retractor devices and systems described herein refers to the section of the retractor that is used to separate tissue. Merely by way of example, FIGS. 44A and 44B depict a top-down view of tissue retractor blades 11001a and 11001b of tissue retractor blade system 11000, and FIGS. 45A & B depict tissue retractor blades 12001a and 12001b of tissue retractor blade system 12000. Each tissue retractor blade depicted has a tissue facing side and a cavity forming side. The cavity forming side of the tissue retractor blades face one another, and a cavity is formed or enlarged when the retractor blades are separated from one another. The second features common to many of the tissue retractors described herein are a pair of “medial bridges,” which function to prevent medial-lateral tissue encroachment. For example, FIG. 44B depicts tissue retractor blade system 11000 with tissue retractor blades 11001a and 11001b separated by and joined together through medial bridges formed by tracks 11002a and 11002b. Similarly, FIG. 45B depicts tissue retractor system 12000 with hinged/folding bars 12002a and 12002b, which each serve as medial bridges to connect tissue retractor blades 12001a and 12001b, and therefore prevent tissue encroachment when deployed. A final common feature to all of the tissue retractors described herein is a point of attachment between each arm of the tissue retractor systems and a tissue retractor blade. An example of this point of attachment is shown in FIG. 51B, which shows tissue retractor blade 18001a attaching to traveling arm 18015 through dowel pin 18002a and ball-nose spring plunger 18003a.

Provided below are additional descriptions of various components, combinations of components, and configurations of components relative to one another that can be used to arrive at each of a number of embodiments of innovative tissue retractor systems. Additional features that can be included in the tissue retractor blades are also described.

### Tissue Retractor Blades

Various shapes of tissue retractor blades are within the scope of the present invention. Merely by way of example, the tissue retractor blades may be flat, curved to varying degrees, angled (at practically any angle) or combinations thereof. In various embodiments, the tissue retractor blades of the present invention may be made of any suitable material including, but in no way limited to metals such as titanium, surgical steel, ceramic, stainless steel, structural plastics such as polycarbonate, Nylon, HDPE, PEEK, etc. In some embodiments, the tissue facing sides of one or more tissue retractor blades are textured for improved traction and stability when they are inserted into tissue at a site of interest. In some embodiments, the texture is formed in (i.e. recessed in) one or more tissue retractor blade. In some embodiments, the texture is formed on (i.e. elevated from) one or more tissue retractor blade. In certain embodiments, the texture is in the form of a pattern (e.g., one or more chevron shape). In certain embodiments, the texture is retractable and/or extendable by one or more mechanism embedded in one or more retractor blade. In that way, the texture can be extended or retracted after one or more retractor blade is inserted into a tissue site. A non-limiting example of a pattern of chevrons is shown in FIG. 51B.

In some embodiments, the medial bridges of the tissue retractor blades are configured so that each tissue retractor blade can slide along a medial bridge. A non-limiting example of this type of configuration is depicted in FIG. 44. In some embodiments, the medial bridge includes a slot along which a complementary element (e.g., a knob, tab, wheel, peg, and the like) from a tissue facing portion of a retractor blade is configured to slide. In certain embodiments, the length of the medial bridges are greater than the collective diameter of the rounded tissue retractor blades when the tissue retractor blades are touching at the edges of their cavity facing sections, as depicted in FIG. 44A. In other embodiments, the medial bridges may be smaller than the diameter of the rounded tissue retractor blades.

In some embodiments, the medial bridges are hinged, and one or more connection points between the medial bridges and tissue retractor blades are configured to pivot, thereby allowing the medial bridges to fold on themselves and adopt a narrower profile, which advantageously facilitates insertion into a tissue site. In some embodiments, the hinged medial bridges are configured to lock. Thus, the narrower profile folded form of the tissue retractor blades and medial bridges can be inserted into a tissue site and then extended/unfolded in order to provide stable tissue displacement on the tissue facing side of the medial bridges, while increasing the size of the cavity formed by the retractor blades and medial bridges. A non-limiting example of this type of configuration is depicted in FIG. 45B.

In various embodiments, the invention teaches two opposing tissue retractor blades that include protrusions on each edge along the axis of insertion. In some embodiments, the protrusions are designed to fit within channels formed on complimentary medial bridges. One non-limiting example of this type of arrangement is tissue retractor blade system 13000 shown in FIGS. 46A and 46B. In this system, protrusion 13004a of tissue retractor blade 13001a is shown mated with channel 13005a of lateral slider 13002a. Thus a medial bridge is formed by 13002a, which connects 13001a and 13001b. In alternative embodiments, the tissue retractor blades have channels and the medial bridges have protrusions, thus reversing the interaction between the edges of the tissue retractor blades and medial bridges described above, while maintaining the same functionality. In certain embodiments, a medial bridge may be formed by several components that cooperate to make a complete medial bridge of a desired length. For example, a medial bridge may include a long segment 13002a and one or more short segments (e.g., 13003a-13003d) which are connected to one another to form a medial bridge of a length desired for a particular application.

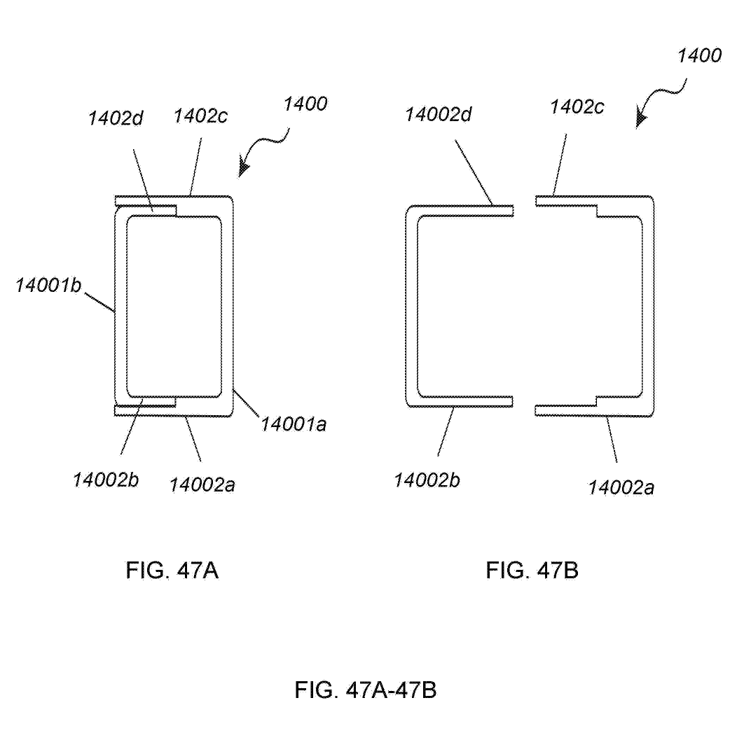

In various embodiments, the tissue retractor blades are designed such that the cavity forming faces of opposing tissue retractor blades nest within one another. In some embodiments, the nesting tissue retractor blades have a rectangular shape. In certain embodiments, the tissue retractor blades nest as depicted in FIG. 47A.

In some embodiments, the tissue facing section of one or more of the tissue retractor blades includes one or more holes through which screws or other fastening components can be inserted to secure one or both tissue retractor blades to an anatomical structure within the body, such as a region of bone. In some embodiments, the bone may be a bone forming a portion of a spine. In some embodiments, the arms of one or more of the tissue retractor blades may be elongated to function as a medial bridge. In these embodiments, even if the opposing blades are separated from one another, the elongated arms allow for added stability, which reduces tissue encroachment in a similar manner as a medial bridge that connects the two opposing blades, as described above. Merely by way of example, retractor system 14000 (FIG. 47B) includes tissue retractor blades with elongated arms that could be of appropriate dimensions to prevent tissue encroachment, even when the blades are somewhat separated. Depending upon the location of insertion, size of incision, and size of the cavity to be formed by the tissue retractor blades, the arms of the tissue retractor blades may be made more or less ridged to allow for effective tissue separation and prevention of tissue encroachment.

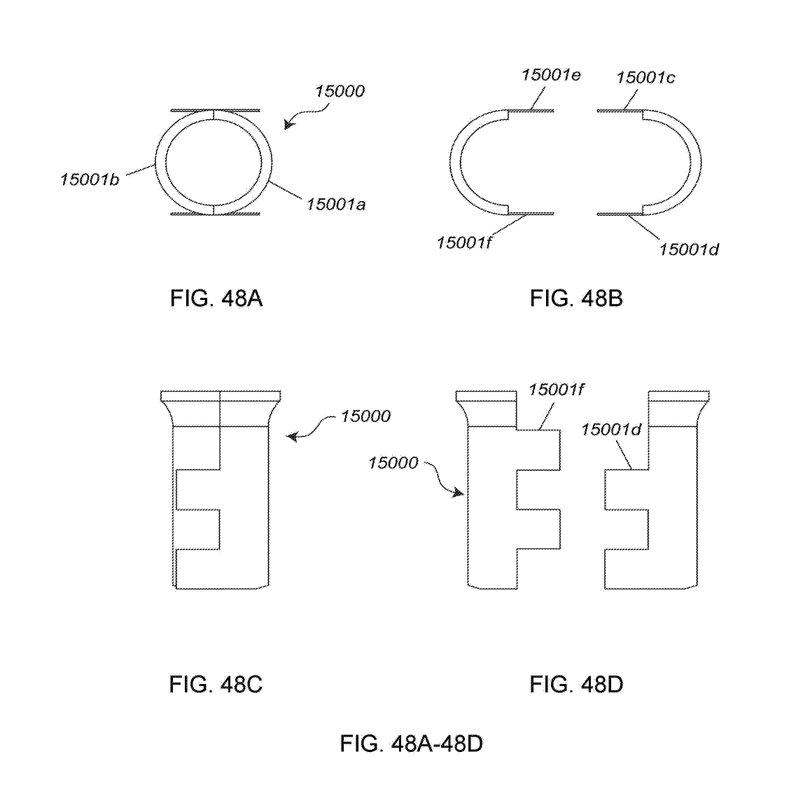

In certain embodiments, the tissue retractor blades include interlocking “fingers” that are configured to interact with one another when the cavity forming side of the pair of tissue retractor blades are brought together. A non-limiting example of tissue retractor blades with an interlocking finger configuration is depicted in FIGS. 48C and 48D. This type of configuration forms medial bridges that can obviate the need for using a separate medial-lateral retractor (e.g. a medial-lateral retractor of the type shown in FIG. 56).

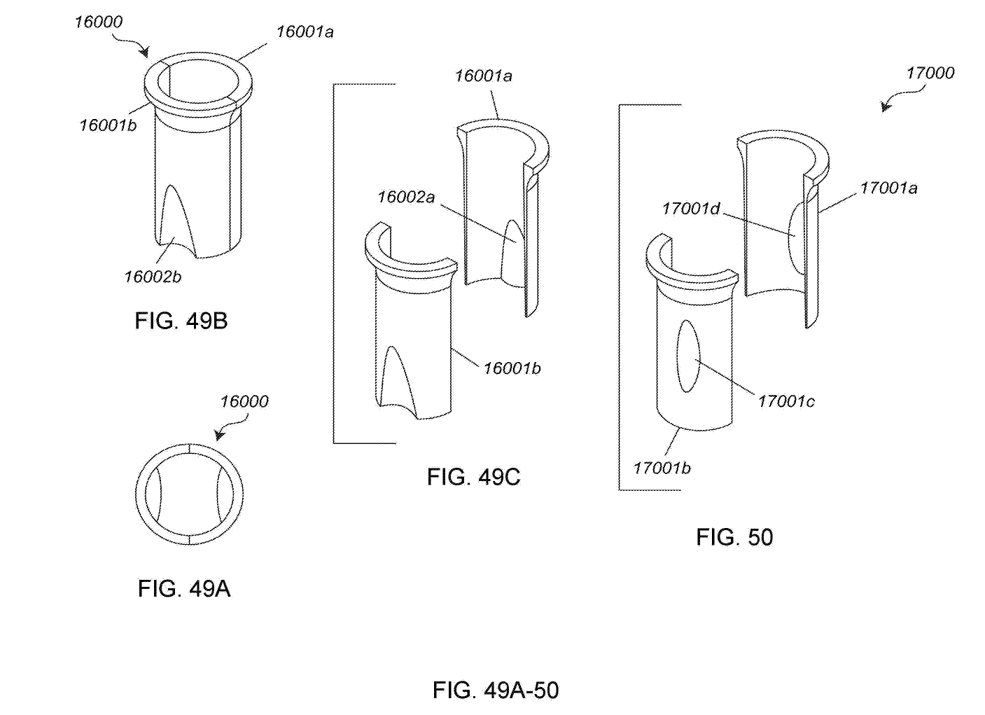

In various embodiments, one or more blades of the tissue retractor system may include a recessed portion designed to accommodate an anatomical structure within a subject's body. In some embodiments, the recessed portion may be parabolic in shape. A non-limiting example of a tissue retractor system with tissue retractor blades that have a parabolic recessed portion is depicted in FIG. 49B. The configuration shown in FIG. 49B is especially useful for procedures performed on or around the spinal cord, as the recessed sections can be configured with appropriate dimensions to accommodate superior and inferior spinal structures.

### Tissue Retractor Arms and Tissue Spreading Components

The tissue retractor blades described in the present application can be permanently or removably attached to retractor arms of a tissue retractor device. There are many ways in which the tissue retractor blades can be attached to retractor arms, including, but in no way limited to, by one or more screw, bolt, tongue and groove arrangement, protrusion (e.g. pin, peg, knob, etc.), clamp, track, and the like. One non-limiting example of a way in which the tissue retractor blades described herein can be attached to tissue retractor arms is depicted in FIG. 51B. FIG. 11B shows tissue retractor blade 18001a is attached to retractor traveling arm 18015 through dowel pin 18002a and ball-nose spring plunger 18003a. FIG. 51B further shows tissue retractor blade 18001b is attached to fixed retractor arm 18004 using the same type of attachment. Although not specifically depicted in each of the referenced drawings, any of the attachment mechanisms described in this section could be used to attach any of the retractor blades described herein to the arms of a tissue retractor (e.g., a tissue retractor described herein or of another type known in the art).

### Tissue Spreading Mechanisms

In operation, each of the embodiments of tissue retractor systems described above depend on modulating the distance between the tissue retractor blades before and/or after the blades are inserted into a tissue site of interest. Thus, the tissue retractor systems described herein may include tissue retractor arms associated with the tissue retractor blades. In some embodiments, the tissue retractor systems are configured such that a first tissue retractor arm (to which one of the tissue retractor blades is connected) is in a fixed position, while a second tissue retractor arm is configured to move relative to the first, much like the components of a crescent wrench.

While there are many ways in which such a configuration can be accomplished, FIGS. 51A and 51B depict exemplary tissue retractor 18000 with traveling retractor arm 18015 and fixed retractor arm 18004. Various adjustment mechanisms may be implemented, such as wing expansion knob 18008 to move the traveling retractor arm 18015 with respect to the fixed retractor arm 18004. For instance adjustment mechanisms may also include, but are not limited, knobs, dials, nuts, ratchet mechanisms, and other suitable adjustment mechanisms. In operation, when wing expansion knob 18008 is rotated, traveling arm 18015 and main arm 18004 are separated from one another to a desired distance, thereby also separating chevron textured tissue retractor blades 18001a and 18001b. In this example, as disclosed herein, the wing expansion knob 18008 (which may be any knob or rotatable implement), is connected to gear teeth that engage a toothed track (rack gear 18005) to move the travelling arm 18015 away and towards fixed arm 18004 along the rack gear 18005. In other examples, the rack gear 18005 could include a screw configuration instead of gear teeth 18017 that allowed rotation of the rack gear 18005 to extend it to the proper position. In some examples, a nut or similar device may contact rack gear 18005 that includes threads for the nut to travel on by rotation of the nut. Rotation of the nut (in a plane perpendicular to the wing expansion knob 18008) could also extend and retract the rack gear 18005. In other examples, the rack gear 18005 may slide along a track. F

Merely by way of example, the FIG. 51A depicts stabilizing arm connection features 18018a and 18018b, which connect to stabilizing tabs by a mating of interlocking tooth face splines fixed together with a threaded stud in a threaded hole.

### Stabilization Arms

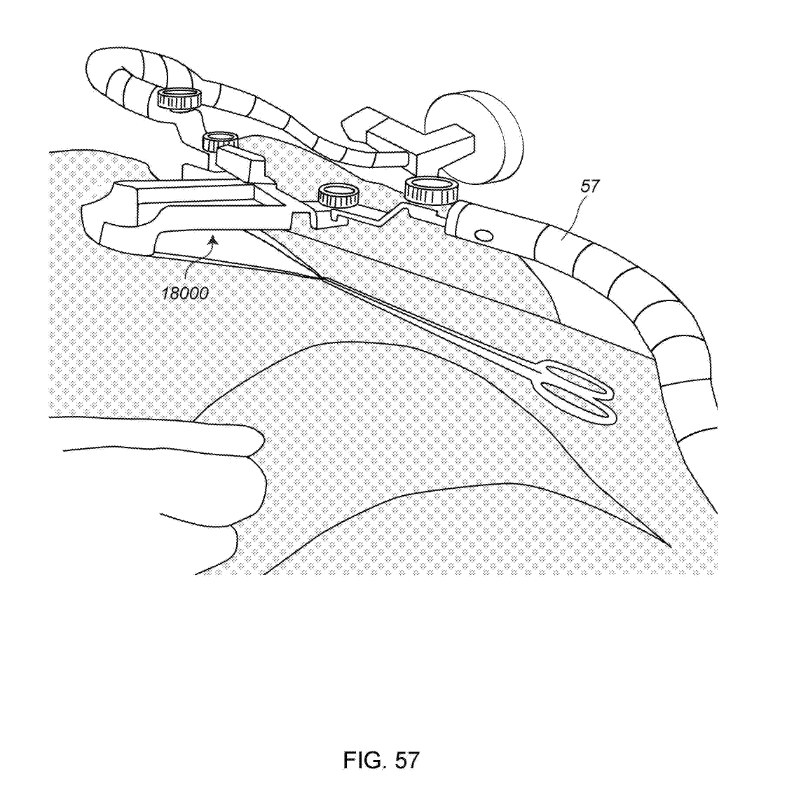

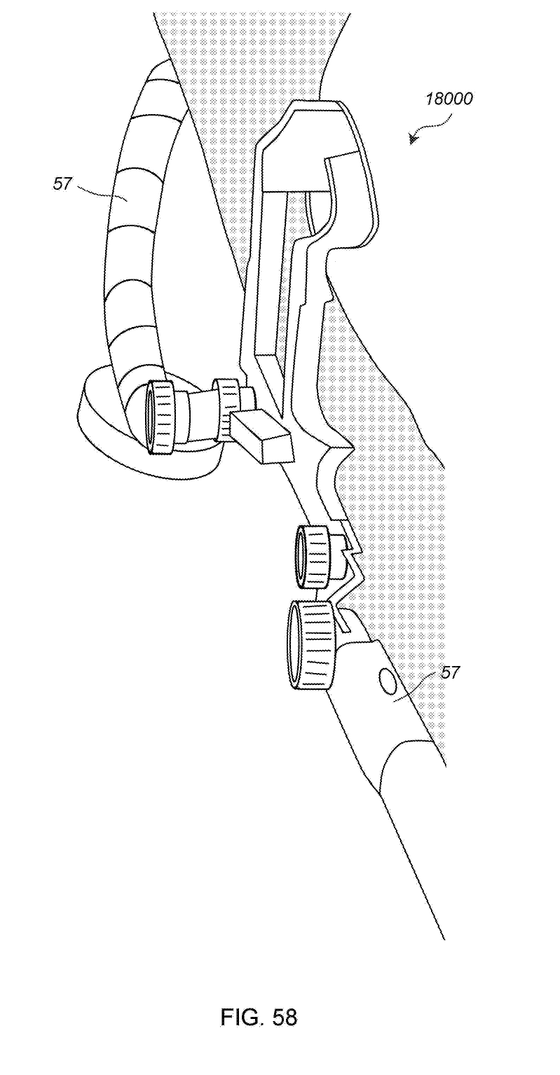

In various embodiments, the tissue retractors of the present invention can be attached to stabilizing arms that can in turn be connected to an operating table or any other stable structure in an operating room. FIG. 57 depicts stabilizing arms 5700 attached to tissue retractor system 18000. Stabilizing arms 5700 may be attached to any tissue retractor system described herein, and may thus form a larger system. The stabilizing arms 5700 may be of any type known in the art, and may be attached to any tissue retractor system described herein by any type of attachment known in the art. For instance, the DORO flexible retractor arms available from PMI Surgical at http://www.pmisurgical.com/de/hirn-retraktoren/doror-flexible-arms/?no_cache=1&sword_list %5B0%5D=cobra may provide a suitable stabilization arm to connect the retractor system to a surgical table.

Integration with Stereotactic Apparatus

A clamping mechanism can be used to attach a stereotactic surgical apparatus to any of the tissue retractor systems described herein. Merely by way of example, the clamping mechanism 5000 shown in FIG. 42 can be used to securely and removably attach a stereotactic apparatus (including stereotactic apparatus 100) to a tissue retractor. Tissue retractor system 18000 may be attached to clamping mechanism 5000 at the position indicated by the arrow in FIG. 11B.

In some embodiments, all or a portion of the apparatuses described herein are made of stainless steel. In some embodiments, the apparatuses are made of titanium, austenitic steel, martensitic steel, brass, carbon fiber, plastic, combinations thereof, and the like. In preferred embodiments, the material or materials used are biocompatible.

In some embodiments, the invention teaches a method that includes using any of the tissue retractor devices or systems described herein for the purposes of facilitating one or more of the processes of (1) introducing a substance into a subject, (2) removing a substance from a subject, and (3) manipulating a portion of a subject's body. One of skill in the art would readily appreciate that the device could be used to spread tissue at almost any location of the body, in order to introduce a substance into and/or remove a substance from any portion of subject's body, including, but in no way limited to an organ, joint (shoulder, hip, knee, etc.), ligament, tendon, muscle, cavity, or any type of tissue. In some embodiments, the substances introduced into the subject's body can include but are in no way limited to biological and/or synthetic substances. Biological substances can include, but are in no way limited to stem cells, neural progenitor cells, tissues, blood, hormones, clotting factors, vectors (including but not limited to viral vectors, plasmids and the like), DNA, RNA, proteins, growth factors, inhibitory substances, matrices, combinations thereof, and the like. Synthetic substances that can be introduced into a subject's body can include but are in no way limited to pharmaceutical agents, markers (including but not limited to biomarkers or any other type of marker that could be visualized with or without the use of imaging equipment), implantable medical devices, electrical sensors, electrical stimulators, glue, sutures, chemotherapeutics, radioactive substances, hyperpolarized substances, combinations thereof, and the like.

In some embodiments, the inventive method includes using any of the inventive tissue retracting systems and apparatuses described herein to facilitate the insertion of any of the instruments described herein. In an embodiment, the inventive method includes using guiding arm 1000 of inventive apparatus 100 to introduce a needle associated with a cannula into any portion of a subject's spinal cord through the space framed by a tissue retractor described herein. A payload of neural progenitor cells is then advanced through the cannula and needle and into the subject's spinal cord.

FIG. 43A depicts an exemplary tissue retractor blade set 10000 with tissue retractor blades 10001a and 10001b positioned close to one another. FIG. 43B depicts tissue retractor blades 10001a and 10001b separated from one another, leaving a gap through which tissue may encroach when the blades are inserted into a tissue site as part of a tissue retractor system, as described above. An example of a tissue retractor with this type of blade design is the Mast Quadrant Retractor™. The distance between the tissue retractor blades is modulated by moving the arms to which the tissue retractor blades are attached (FIG. 41). When medial-lateral tissue retraction is required, an additional medial-lateral tissue retractor can also be attached to the system, as depicted in FIG. 56.

FIG. 44A depicts tissue retractor system 11000, which includes tissue retractor blades 11001a and 11001b, along with sliding medial-lateral blades 11002a and 11002b. The sliding medial-lateral blades 11002a and 11002b include tracks 11003a, 11003b, 11003c, and 11003d that mate with tissue retractor blade ends 11004a, 11004b, 11004c, and 11004d, as shown in FIG. 44B. In operation, medial-lateral blades 11002a and 11002b may be secured after a small amount of initial incision retraction, so that incision size can accommodate their fixed length.

FIG. 45A depicts tissue retractor system 12000, which includes tissue retractor blades 12001a and 12001b, as well as hinged medial-lateral bars 12002a and 12002b (FIG. 45B). FIG. 45B shows the medial-lateral blades 12002a and 12002b extended to give a maximum distance between tissue retractor blades 12001a and 12001b. This tissue retractor blade system eliminates the need for a separate medial-lateral retractor, and reduces the number of additional components of the device. Although both ends of the hinged medial-lateral bars 12002a and 12002b are shown permanently attached to both tissue retractor blades 12001a and 12001b, the tissue retractor system could also be configured such that the medial-lateral blades are only permanently attached to one tissue retractor blade at one end, while the other end removably but stably attaches to the opposing retractor blade.

FIG. 46A depicts tissue retractor system 13000. Tissue retractor system 13000 includes tissue retractor blades 13001a and 13001b, as well as two or more medial-lateral sliding components (“sliders”) 13002a, 13002b, 13003a, 13003b, 13003c, and 13003d. Medial-lateral sliders 13002a and 13002b include receiving sections/channels 13005a, 13005b, 13005c, and 13005d, as shown. Channels 13005a-13005d are designed to mate with protrusions 13004a-13004d of tissue retractor blades 13001a and 13001b, as shown in FIG. 46B. If a medial-lateral slider of a different length is required for a particular procedure, then one or more smaller medial-lateral sliders 13003a-13003d may be linked to a larger medial-lateral slider (i.e., 13001a and 13001b). In operation, the medial-lateral sliders can be inserted into (i.e. dropped into) place after the tissue retractor blades have been inserted into the incision in the subject and separated by an appropriate distance.

FIG. 47A depicts tissue retractor system 14000. Tissue retractor system 14000 includes tissue retractor blades 14001a and 14001b with rigid projections 14002a, 14002b, 14002c, and 14002. FIG. 47A depicts nesting projections 14002a, 14002b, 14002c, and 14002d overlapping when tissue retractor blades 14001a and 14001b are in close proximity. FIG. 47B depicts tissue retractor blades 14001a and 14001b separated from one another. Importantly, because of the length of rigid projections 14002a-14002d, tissue encroachment is prevented even when retractor blades 14001a and 14001b are separated by a reasonable distance.

FIGS. 48A and 48B depict a top view of tissue retractor system 15000, which includes tissue retractor blades 15001a and 15001b, which include interlocking finger projections 15001c, 15001d, 15001e, and 15001f. FIGS. 48C and 48D show interlocking finger projections 15001d and 15001f, and 15001e and 15001c (hidden from view) mated with one another and then separated from one another.

FIGS. 49A-49C depict alternate views of tissue retractor system 16000 with tissue retractor blades 16001a and 16001b, which include parabolic indentations 16002a and 16002b, respectively. Parabolic indentations 16002a and 16002b are designed to accommodate the superior and inferior spineous processes.

FIG. 50 depicts a perspective view of tissue retractor system 17000 with tissue retractor blades 17001a and 17001b and retractor blade openings 17001c and 17001d.

FIG. 51A depicts a perspective view of tissue retractor system 18000, in which tissue retractor blades 18001a and 18001b are shown in close proximity. FIG. 51B depicts an exploded view of tissue retractor system 18000. Tissue retractor system 18000 includes tissue retractor blades 18001a and 18001b, which are attached to traveling arm 18015 and fixed arm 18004, respectively. Tissue retractor blades 18001a and 18001b include projections 18001c, 18001d, 18001e, and 18001f (hidden from view but parallel to 18001c on retractor blade 18001a).

Retractor blade 18001a is attached to traveling arm 18015 through dowel pin 18002a and ball-nose spring plunger 18003a (analogous attachment of retractor blade 18001b to fixed arm 18004 through dowel pin 18002b and ball-nose spring plunger 18003b is also shown). FIG. 51B further shows retractor ratchet lock 18016 and spring 1804 which engage traveling arm 18015. Expansion wing knob 18008 is attached to travelling arm 18015 through flange bushing 18009 and retractor pinion shaft 18010.

Expansion wing knob 18008 engages the travelling arm 18015, in some examples by a gear mechanism that includes a gear connected to the retractor pinion shaft 18010. Rotation of the expansion wing knob 18008 causes the teeth on the retractor pinion shaft 18010 to contact the teeth 18017 on the rack gear 1805, moving the rack gear 18005 and causing the traveling arm 18015 to either extend from the fixed arm 18004 or retract towards the fixed arm 18004. Accordingly, the rack gear 1805, teeth 18017 and the teeth on the reactor pinion shaft 18010 provide a system for moving the travelling arm 18015 away and towards the fixed arm 18004. Retractor ratchet lock 18016 in some examples may lock the travelling arm 18015 into place. Accordingly, by pulling the lever on retractor ratchet lock 18016 out, a lock pin 18014 or other mechanism may be removed from contacting the gear teeth 18017 and allow the rack gear 18017 to be moved by rotation of the wing knob 18008.

Accordingly, using the gear system, the travelling arm 18015 may be extended at a steady finely tuned pace using the wing knob 18008. For instance, the wing knob 18008 allows for finely tuned adjustments as rotation of the wing knob 18008 provides for relatively small displacements of the travelling arm 18015 in some examples. In other examples, other suitable devices may be utilized to mechanically extend the travelling arm 18015 from the fixed arm 18004 with a fine level of motor control that allows the tissue to be retracted to an optimum amount without causing damage. Additionally, the gear mechanism provides an easy method to keep the travelling arm 18015 in place once it is extended to the desired length.

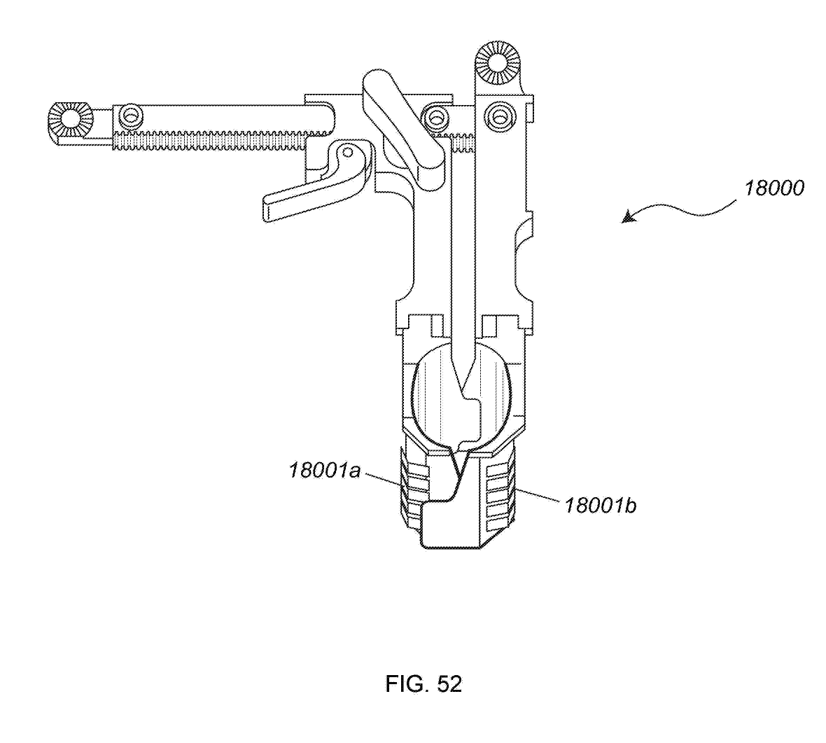

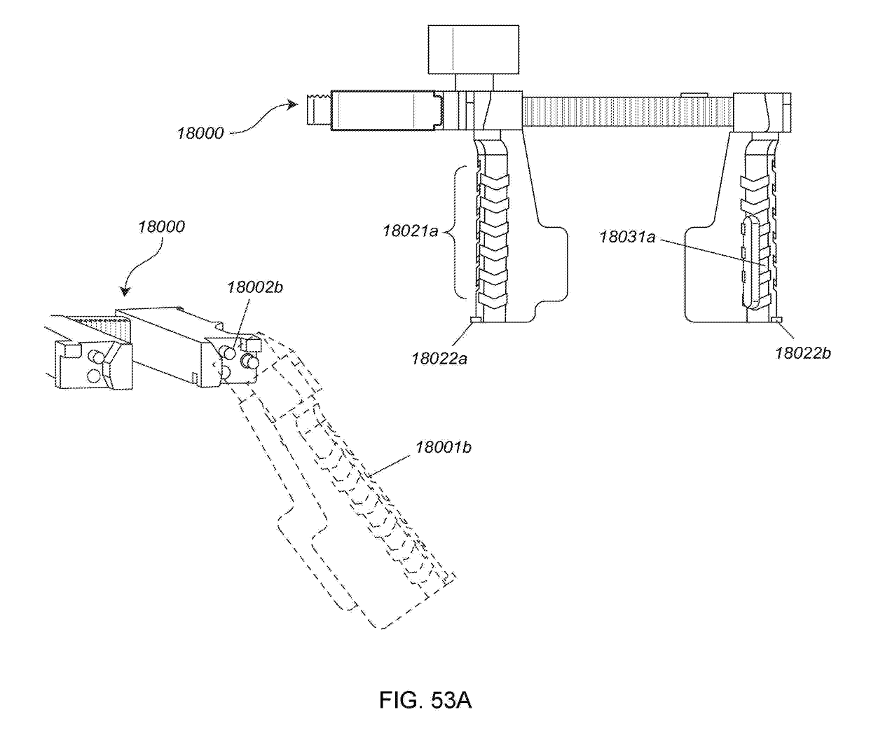

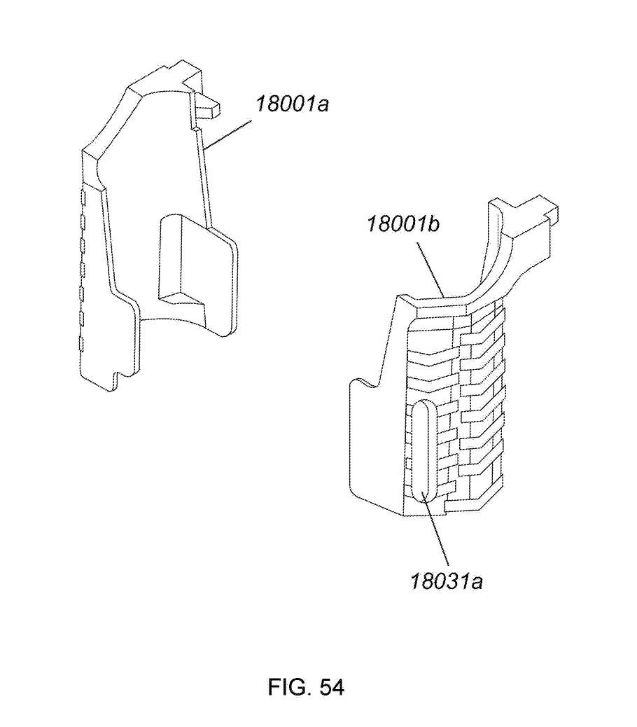

Retractor 18000 with stabilizing arm connection feature 18018b can be seen engaged in traveling arm 18015 (FIG. 51A). Travel stops 18007a and 18007b can also be seen engaged in retractor rack gear 18005 in FIG. 51A. Fixed arm 18004 accommodates rack gear 18005 through channel 1821. Rack gear 18005 is secured in place through bolt 18006. Fixed arm 18004 further includes stabilizing arm connection feature 18018a. Chevron-patterned texture 18021a on tissue retractor blade 18001a can also be seen (the same pattern is also present on tissue retractor blade 18001b). Tissue undercut teeth 18022a and 18022b, which provide improved tissue traction, are shown in FIG. 11B. FIG. 52 depicts an alternate view of tissue retractor system 18000, in which tissue retractor blades 18001a and 18001b are shown interlaced. FIG. 53A depicts an alternate view of tissue retractor system 18000, which shows sloped side walls/projections of tissue retractor blades 18001a and 18001b. This feature allows greater access for a surgeon when performing procedures within the cavity formed by the tissue retractor blades. FIG. 53A also shows rotation stops 18030a and 18031a. FIG. 53B shows blade nesting slot 18031a on tissue retractor blade 18001b. A similarly situated nesting slot is situated on a parallel section of tissue retractor blade 18001b (not shown). FIG. 54 depicts an alternate view of tissue retractor blades 18001a and 18001b and nesting slot 18031.

In operation, retractor blades 18001a and 18001b of tissue retractor system 18000 are placed into an incision in a subject and locked into place with surgical stabilization arms that interact with stabilization arm connection features 18018a and 18018b, as described above. Turning the expansion wing knob of the tissue retractor in a clockwise direction drives retractor blade 18001a away from retractor blade 18001b, thereby retracting the subject's tissue and creating an accessible cavity. The blades of tissue retractor 18000 spread apart from one another until they reach a maximum distance of 58 mm (although retractor systems of other dimensions could be made according to the same principles described herein without departing from the spirit of the invention). In certain procedures, a stereotactic device can then be attached to the tissue retractor system, as shown in FIGS. 41 and 42. The mounting interface for the stereotactic device is a horizontally-oriented straight rectangular cross-section member of the retractor main arm that is 8 mm tall by 10 mm wide in this particular embodiment. The region for mounting the stereotactic device 100 is 24.5 mm long, allowing space for the mounting clamp of the stereotactic device to engage and grip the retractor firmly, as shown in FIGS. 41 and 42. To mount retractor blades 18001a and 18001b, the user must rotate the desired blade outwards approximately 35° before sliding the blade onto the retractor arm guide pin 18002b, as shown in FIG. 53A. As the faces of the mounting features on the retractor blade and arm (main arm or traveling arm) come together the blade can be rotated down/inwards to a vertical orientation. As this rotation occurs, a hook feature engages with the arm overhang, preventing the blade from pulling straight off. The guide pin 18002b holds the blade vertically, and rotation stops prevent the blade from collapsing inwards under the load of retracted tissue. The octagonal outer surface (tissue facing) of retractor blades 18001a and 18001b allow for easier insertion than squared blades, and aids in incision compliance as the tissue moves around the blades during retraction.

### SELECTED EMBODIMENTS

Although the above description and the attached claims disclose a number of embodiments of the present invention, other alternative aspects of the invention are disclosed in the following further embodiments.

### Embodiment 1

An imaging system comprising: a tissue retractor comprising a first blade and a second blade; a radiation source coupled to the first blade of the tissue retractor; and a radiation detector coupled to the second blade of the tissue retractor.

### Embodiment 2

The imaging system of embodiment 1, further comprising a collimating device configured to collimate the radiation emitted from the radiation source.

### Embodiment 3

The imaging system of embodiment 2, wherein the radiation source is an ionizing radiation source comprising argon.

### Embodiment 4

The imaging system of embodiment 3, wherein the radiation detector is a digital radiation detector.

### Embodiment 5

The imaging system of embodiment 4, wherein each of the retractor blades is connected to a retractor arm.

### Embodiment 6

The imaging system of embodiment 5, further comprising a stereotactic device attached to one of the retractor arms, wherein the stereotactic device comprises an elongated guiding arm capable of advancing towards and retracting from a tissue site of a subject.

### Embodiment 7

The imaging system of embodiment 6, further comprising a cannula system, wherein the cannula system is attached to the guiding arm of the stereotactic device, and wherein the cannula system comprises a hollow needle and a tube connected thereto.

### Embodiment 8

The imaging system of embodiment 7, further comprising a syringe pump, wherein the syringe pump is operably connected to and in fluid communication with the tube of the cannula system, and wherein the syringe pump is attached to the stereotactic device.

### Embodiment 9

The imaging system of embodiment 8, further comprising a computing system operably connected to the ionizing radiation source and/or the digital radiation detector, wherein the computing system comprises a processor configured to process data acquired by utilizing the ionizing radiation source and the digital radiation detector.

### Embodiment 10

The imaging system of embodiment 9, wherein the computing system is wirelessly connected to the ionizing radiation source and/or the digital radiation detector.

### Embodiment 11

The imaging system of embodiment 10, wherein the computing system is connected to the ionizing radiation source and/or the digital radiation detector through one or more wires.

### Embodiment 12

A method comprising: (1) providing an imaging system comprising: a tissue retractor comprising a first blade and a second blade; a radiation source coupled to the first blade of the tissue retractor; and a radiation detector coupled to the second blade of the tissue retractor; (2) positioning each of the retractor blades of the imaging system on opposing sides of an anatomical structure of a subject; and (3) imaging the anatomical structure by operating the radiation source and the radiation detector.

### Embodiment 13

The method of embodiment 12, wherein the imaging system further comprises a collimating device configured to collimate the radiation emitted from the radiation source.

### Embodiment 14

The method of embodiment 13, wherein the radiation source of the imaging system is an ionizing radiation source comprising argon.

### Embodiment 15

The method of embodiment 14, wherein the radiation detector of the imaging system is a digital radiation detector.

### Embodiment 16

The method of embodiment 15, wherein each of the retractor blades of the imaging system is connected to a retractor arm.

### Embodiment 17

The method of embodiment 16, wherein the imaging system further comprises a stereotactic device attached to one of the retractor arms, and wherein the stereotactic device comprises an elongated guiding arm capable of advancing into and retracting away from a tissue site on a subject.

### Embodiment 18

The method of embodiment 17, wherein the imaging system further comprises a cannula, and wherein (1) the cannula is attached to the guiding arm of the stereotactic device, and (2) the cannula comprises a hollow needle and a tube connected thereto.

### Embodiment 19

The method of embodiment 18, wherein the imaging system further comprises a syringe pump, and wherein (1) the syringe pump is operably connected to and in fluid communication with the tube of the cannula, and (2) the syringe pump is attached to the stereotactic device.

### Embodiment 20

The method of embodiment 19, wherein the imaging system further comprises a computing system operably connected to the ionizing radiation source and/or the digital radiation detector, and wherein the computing system comprises a processor configured to process data acquired by utilizing the ionizing radiation source and detector.

### Embodiment 21

The method of embodiment 20, wherein the computing system is wirelessly connected to the ionizing radiation source and/or the digital radiation detector.

### Embodiment 22

The method of embodiment 21, wherein the computing system is connected to the ionizing radiation source and/or digital radiation detector through one or more wires.

### Embodiment 23

The method of embodiment 22, wherein the anatomical structure is the subject's spinal cord.

### Embodiment 24

The method of embodiment 23, further comprising introducing the needle of the cannula system into the subject's spinal cord by advancing the guiding arm of the stereotactic device toward the subject's spinal cord.

### Embodiment 25

The method of embodiment 24, further comprising operating the syringe pump to pump a composition comprising cells into the subject's spinal cord through the needle.

### Embodiment 26

The method of embodiment 25, wherein the cells comprise neural progenitor cells.

### Embodiment 27

The method of embodiment 26, wherein the neural progenitor cells express glial cell line derived neurotrophic factor.

### Embodiment 28

The method of embodiment 27, wherein the subject has been diagnosed with amyotrophic lateral sclerosis.

### Embodiment 29

A kit comprising the imaging system of any of embodiments 1-11 and instructions for the use thereof to image an anatomical structure of a subject.

### Embodiment 30

A tissue retractor system comprising: a fixed tissue retractor arm with a first tissue retractor blade attached thereto; a movable tissue retractor arm with a second tissue retractor blade attached thereto; a gear rack mechanically connected to the fixed tissue retractor arm and the movable retractor arm; and a mechanism configured to actuate the position of the movable tissue retractor arm, wherein the first tissue retractor blade and the second tissue retractor blade each comprise at least 3 elongated walls.

### Embodiment 31

The tissue retractor system of embodiment 1, wherein the first and second tissue retractor blades are approximately U-shaped.

### Embodiment 32

The tissue retractor system of embodiment 1 wherein one or more elongated walls of the first and/or second tissue retractor blades comprise one or more ridges.

### Embodiment 33

The tissue retractor system of embodiment 3, wherein the one or more ridges are part of a pattern.

### Embodiment 34

The tissue retractor system of embodiment 4, wherein one or more of the ridges are in the shape of a chevron.

### Embodiment 35

The tissue retractor system of embodiment 1, wherein the second tissue retractor blade comprises (1) a first vertical slit formed in a first elongated wall and configured to receive a first edge of a first wall of the first tissue retractor blade, and (2) a second vertical slit in a second elongated wall configured to receive a second edge of a second wall of the second tissue retractor blade.

### Embodiment 36

The tissue retractor system of embodiment 1, wherein a section of one or more of the three walls of the first and/or second tissue retractor blades are tapered.

### Embodiment 37

The tissue retractor system of embodiment 1, wherein the gear rack comprises one or more travel stops configured to limit the motion of the movable tissue retractor arm beyond a certain position on the gear rack.

### Embodiment 38

The tissue retractor system of embodiment 1, wherein the movable tissue retractor arm further comprises a back drive prevention lever, wherein the back drive prevention lever terminates in a tooth configured to engage in the gear rack and prevent slipping of the movable tissue retractor arm when the back drive prevention lever is not depressed.

### Embodiment 39

The tissue retractor system of embodiment 1, wherein the gear rack further comprises an attachment mechanism configured to attach the gear rack to a first stabilizing arm.

### Embodiment 40

The tissue retractor system of embodiment 10, wherein the fixed tissue retractor arm further comprises an attachment mechanism configured to attach the gear rack to a second stabilizing arm.

### Embodiment 41

The tissue retractor system of embodiment 1, wherein one or more of the tissue retractor blades comprises one or more teeth configured to interact with a tissue of a subject in whom the retractor blades have been inserted.

### Embodiment 42

The tissue retractor system of embodiment 11, wherein the first stabilizing arm is attached to the attachment mechanism of the gear rack configured to attach thereto.

### Embodiment 43

The tissue retractor system of embodiment 13, wherein the second stabilizing arm is attached to the attachment mechanism of the fixed retractor arm configured to attach thereto.

### Embodiment 44

A method comprising forming an incision in a section of tissue within a subject, and inserting the first and second tissue retractor blades of the tissue retractor system of any of embodiments 1-14 into the incision.

### Embodiment 45

The method of embodiment 15, further comprising attaching a stereotactic device to the tissue retractor system.

### Embodiment 46

A kit comprising the tissue retractor system of any of embodiments 1-14 and instructions for inserting the first and second tissue retractor blades of the tissue retractor system into an incision in a subject.

### CONCLUSIONS

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein, including the best mode known to the inventors for carrying out the application. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A tissue retractor system comprising:
a fixed tissue retractor arm with a first tissue retractor blade attached thereto, the first tissue retractor blade comprising three elongated walls;
a travelling tissue retractor arm with a second tissue retractor blade attached thereto, the second tissue retractor blade comprising three elongated walls;
a rack gear connected to the fixed tissue retractor arm and the travelling retractor arm, the travelling tissue retractor arm configured to move along the rack gear; and
an adjustment mechanism in contact with the rack gear configured to move the travelling tissue reactor arm with respect to the fixed tissue retractor arm along the rack gear.

2. The tissue retractor system of claim 1, wherein the first and second tissue retractor blades are approximately U-shaped.

3. The tissue retractor system of claim 1 wherein at least one of the three elongated walls of the first and second tissue retractor blades comprise at least one ridge.

4. The tissue retractor system of claim 3, wherein the at least one ridge comprises a pattern of ridges.

5. The tissue retractor system of claim 4, wherein the pattern of ridges is in the shape of a chevron.

6. The tissue retractor system of claim 1, wherein the second tissue retractor blade comprises:
a first vertical slit formed in a first elongated wall and is configured to receive a first edge of a first wall of the first tissue retractor blade; and
a second vertical slit in a second elongated wall configured to receive a second edge of a second wall of the second tissue retractor blade.

7. The tissue retractor system of claim 1, wherein a section of at least one of the at least three elongated walls of the first and second tissue retractor are tapered.

8. The tissue retractor system of claim 1, wherein the gear rack comprises at least one travel stop configured to limit the motion of the travelling tissue retractor arm beyond a certain position on the rack gear.

9. The tissue retractor system of claim 1, wherein the travelling tissue retractor arm further comprises a back drive prevention lever, wherein the back drive prevention lever terminates in a tooth configured to engage in the rack gear and prevent slipping of the movable tissue retractor arm when the back drive prevention lever is not depressed.

10. The tissue retractor system of claim 1, wherein the gear rack further comprises an attachment mechanism configured to attach the rack gear to a first stabilizing arm.

11. The tissue retractor system of claim 10, wherein the fixed tissue retractor arm further comprises an attachment mechanism configured to attach the gear rack to a second stabilizing arm.

12. The tissue retractor system of claim 11, wherein the first stabilizing arm is attached to the attachment mechanism of the gear rack configured to attach thereto.

13. The tissue retractor system of claim 12, wherein the second stabilizing arm is attached to the attachment mechanism of the fixed retractor arm configured to attach thereto.

14. A method comprising forming an incision in a section of tissue within a subject, and inserting the first and second tissue retractor blades of the tissue retractor system of claim 1 into the incision.

15. The method of claim 15, further comprising attaching a stereotactic device to the tissue retractor system.

16. A kit comprising the tissue retractor system of claim 1 and instructions for inserting the first and second tissue retractor blades of the tissue retractor system into an incision in a subject.

17. A tissue retractor system comprising:
a fixed tissue retractor arm attached to a first tissue retractor blade;
a travelling tissue retractor arm attached to a second tissue retractor blade;
a rack gear connected to the fixed tissue retractor arm and the travelling retractor arm, the travelling tissue retractor arm configured to move along the rack gear; and
an adjustment mechanism in contact with the rack gear configured to move the travelling tissue reactor arm with respect to the fixed tissue retractor arm along the rack gear.

18. The tissue retractor system of claim 17, wherein the adjustment mechanism comprises a at least one of a knob, dial, wing knob, and nut.

19. The tissue retractor system of claim 17, wherein the rack gear comprises gear teeth.

20. The tissue retractor system of claim 19, wherein the adjustment mechanism comprises teeth that contact the gear teeth.

21. A method of injecting a therapeutic substance into an open surgical site of a subject, the method comprising:
providing a tissue retractor with at least two blades,
providing a stereotactic device connected to the tissue retractor, the stereotactic device comprising a guiding section to which an instrument is attached;
inserting the at least two blades into the open surgical site;
actuating a mechanism on the tissue retractor to move the at least two blades relative to each other to hold open the surgical site;
positioning the guiding section so that the instrument is positioned over a tissue site in the open surgical site;
using the instrument to deposit the therapeutic substance in the tissue site.

22. The method of claim 21, wherein the instrument comprises a cannula.

23. The method of claim 21, wherein the therapeutic substance comprises neural progenitor cells.

24. The method of claim 21, wherein the tissue site is the subject's spinal cord.

25. The method of claim 24, wherein the neural progenitor cells express glial cell line derived neurotropic factor.

26. The method of claim 21, wherein the subject has been diagnosed with a neurologic disease, neurologic trauma, cancer, or combinations thereof.

27. The method of claim 21, wherein the subject has been diagnosed with amyotrophic lateral sclerosis.

28. The method of claim 21, wherein the therapeutic substance is deposited through the instrument from a syringe pump.

29. The method of claim 21, wherein the at least two blades include at least one ridge.

30. The method of claim 29, wherein the at least one ridge comprises a pattern of ridges.

31. A method of injecting a therapeutic substance into an open surgical site of a subject that has been diagnosed with amyotrophic lateral sclerosis, the method comprising:
providing a tissue retractor connected to a device configured to inject progenitor cells;
positioning the device over a tissue site of the subject's spinal cord; and
injecting progenitor cells into the tissue site with the device.
